(19) United States
(12) Patent Application Publication
(10) Pub. No.: US 2021/0382037 A1
(43) Pub. Date: Dec. 9, 2021

(54) IDENTIFICATION AND USE OF CYTOTOXIC T LYMPHOCYTE (CTL) ANTIGEN-SPECIFIC TARGET CELL KILLING ENHANCER AGENTS

(71) Applicant: DANA-FARBER CANCER INSTITUTE, INC., Boston, MA (US)

(72) Inventors: Patrick H. LIZOTTE, Boston, MA (US); Paul T. KIRSCHMEIER, Basking Ridge, NJ (US); Mark BITTINGER, Dover, MA (US); Nathanael GRAY, Boston, MA (US)

(73) Assignee: DANA-FARBER CANCER INSTITUTE, INC., Boston, MA (US)

(21) Appl. No.: 16/629,930

(22) PCT Filed: Jul. 9, 2018
(86) PCT No.: PCT/US2018/041266

§ 371 Date: Jan. 9, 2020

Related U.S. Application Data
(60) Provisional application No. 62/530,648, filed on Jul. 10, 2017.
(60) Provisional application No. 62/582,678, filed on Nov. 7, 2017.

(51) Int. Cl. G01N 33/50; C07K 14/47; C07K 16/28

(52) U.S. Cl. CPC G01N 33/505; G01N 33/5014; A61K 38/00; C07K 16/2818; C07K 16/2878; C07K 14/4703

(57) ABSTRACT

The present invention relates to screening methods for identification of agents (e.g., small molecules) that modulate cytotoxic T lymphocyte antigen-specific target (e.g., tumor) cell killing, as well as to uses of compounds identified thereby as immunomodulatory, including use of EGFR inhibitors as immunomodulatory agents.

### RELATED APPLICATIONS

This application is a national stage application, filed under 35 U.S.C. § 371, of International Application No. PCT/US2018/041266, filed Jul. 9, 2018, which claims the benefit of priority under 35 U.S.C. § 119(e) to U.S. Provisional Application No. 62/530,648, filed Jul. 10, 2017 and to U.S. Provisional Application No. 62/582,678, filed Nov. 7, 2017, each of which is incorporated herein by reference in its entirety.

### FIELD OF THE INVENTION

The invention relates generally to methods for identification of immunomodulatory agents and uses of agents identified thereby.

### BACKGROUND OF THE INVENTION

In the mammalian immune system, CD8⁺ cytotoxic T lymphocytes can exert toxicity upon target cells that present major histocompatibility complex type I (MHC-I)-displayed antigens. Agents that either enhance or inhibit the interaction between CD8⁺ cytotoxic T cells and antigen-presenting target cells are attractive for development as immunomodulatory therapeutics. A need exists for additional immunomodulatory therapeutic lead agents, and for development of assays capable of identifying test agents as immunomodulatory therapeutic leads in an efficient and high-throughput manner.

### BRIEF SUMMARY OF THE INVENTION

Described herein is the identification of epidermal growth factor receptor (EGFR) as an immune-oncology target. The current disclosure relates to discovery and development of a high-throughput screening assay for identification of immunomodulatory therapeutic agents, and to EGFR inhibitory agents as agents identified by the screen as possessing the ability to enhance CD8⁺ cytotoxic T lymphocyte-mediated killing of target cells that display MHC-I antigens. Therapeutic use of such immunomodulatory therapeutic lead agents is also described.

In one aspect, the instant disclosure provides a method for identifying an agent capable of modulating the interaction between a CD8⁺ T cell and a cell expressing a model antigen peptide that involves (A) contacting a first population of cells comprising a mixture of (1) cells expressing a model antigen peptide and a first reporter peptide and (2) cells that express a second reporter peptide and do not express the model antigen peptide, with a test agent; (B) assessing expression of the first reporter peptide, the second reporter peptide, or both the first and second reporter peptides, in the first cell population, as compared to an appropriate control cell population expressing the reporter peptide(s) and not contacted with the test agent; (C) contacting a second population of cells comprising a mixture of (1) CD8⁺ T cells; (2) cells expressing the model antigen peptide and the first reporter peptide; and (3) cells that express the second reporter peptide and do not express the model antigen peptide, with the test agent; (D) assessing expression of the first and second reporter peptides in the second cell population, as compared to an appropriate control cell population not contacted with the test agent and expressing the first and second reporter peptides, and (E) identifying the test agent as an agent that modulates CD8⁺ T cell killing of the cells expressing the model antigen peptide if the test agent (a) is not identified to modulate expression of the first reporter peptide, the second reporter peptide, or both the first and second reporter peptides in the first cell population, as compared to the appropriate control cell population expressing the reporter peptide(s) and not contacted with the test agent; and (b) is identified to significantly increase or significantly decrease expression of the first reporter peptide relative to the second reporter peptide in the second population of cells, as compared to the appropriate control cell population not contacted with the test agent and expressing the first and second reporter peptides, thereby identifying the test agent as an agent capable of modulating the interaction between a CD8⁺ T cell and a cell expressing a model antigen peptide.

In one embodiment, the cell expressing a model antigen peptide is an ovarian cancer cell. Optionally, the ovarian cancer cell harbors a nucleotide sequence encoding for the model antigen peptide, operably linked to nucleotide sequence encoding for the first reporter peptide.

In another embodiment, the (1) cells expressing a model antigen peptide and a first reporter peptide and (2) cells that express a second reporter peptide and do not express the model antigen peptide, are derived from the same source cell line, optionally where the source cell line is an ovarian cancer cell line, optionally ID8 cells. In another embodiment, the source cell line is a colon cancer cell line, optionally CT26 cells.

In certain embodiments, the CD8⁺ T cell is an OT-I T cell receptor transgenic cell.

Optionally, the first population of cells, the second population of cells, or both the first and second populations of cells are in an array, optionally in a 96 well plate format.

In one embodiment, in the first population of cells, there is an about 1:1 proportion of (1) cells expressing a model antigen peptide and a first reporter peptide to (2) cells that express a second reporter peptide and do not express the model antigen peptide. In certain embodiments, there is at least about a 2:10 proportion of (1) CD8⁺ T cells to (2) cells expressing the model antigen peptide and the first reporter peptide, optionally about a 3:10 to about a 10:1 proportion of (1) CD8⁺ T cells to (2) cells expressing the model antigen peptide and the first reporter peptide, optionally about a 1:1 to about a 2:1 proportion of (1) CD8⁺ T cells to (2) cells expressing the model antigen peptide and the first reporter peptide.

In certain embodiments, the first reporter peptide or the second reporter peptide is firefly luciferase. Optionally, the second reporter peptide or the first reporter peptide is renilla luciferase. In some embodiments, the first reporter peptide is firefly luciferase and the second reporter peptide is renilla luciferase.

In one embodiment, the test agent is identified as an agent that modulates the viability of the first population of cells if the expression of the reporter peptide(s) is significantly increased or significantly reduced in the first population of cells, as compared to an appropriate control cell population. In a related embodiment, the test agent is identified as an agent that reduces the viability of the first population of cells if the expression of the reporter peptide(s) is reduced by at least about two-fold in the first population of cells, as compared to an appropriate control cell population, optionally where the appropriate control cell population is a cell population not contacted with a test agent, optionally where the appropriate control cell population is contacted with dimethyl sulfoxide (DMSO).

In certain embodiments, the first population of cells and the second population of cells are contacted under standard mammalian cell culture growth conditions, optionally at 37° C. and 5% O₂.

In some embodiments, the first population of cells and the second population of cells are grown and/or contacted under one or more of the following conditions: hypoxic conditions, in the presence of hydrogen peroxide, in the presence of transforming growth factor beta (TGF-β) and/or interleukin-10 (IL-10), in the presence of T regulatory cells, in the presence of MDSCs (myeloid-derived suppressor cells), in the absence of L-arginine and/or in the absence of L-cysteine.

In certain embodiments, at least one of the assessing steps is performed at between 12 h and 72 h after the first population of cells or the second population of cells is contacted with test agent, optionally where the at least one of the assessing steps is performed at about 48 h after the first population of cells or the second population of cells is contacted with test agent, optionally where the assessing steps are performed at about 48 h after the first population of cells is contacted with test agent and at about 48 h after the second population of cells is contacted with test agent, respectively.

In one embodiment, the test agent is a small molecule. Optionally, the test agent is a kinase inhibitor. In certain embodiments, the test agent is one of the following: Seliciclib ((R)-Roscovitine; CYC202; target=CDK2); ALW-II-38-3 (target=DDR1); ALW-II-49-7 (target=DDR1); AT-7519 (target=CDK9); Tivozanib (AV-951; target=VEGFR-2); AZD7762 (target=CHK1); AZD8055 (target=mTOR); Sorafenib (BAY-439006; target=BRAF); CP466722 (target=ATM); CP724714 (target=erbB-2); Alvocidib (Flavopiridol; HMR-1275; L868275; target=CDK1); GSK429286A (target=ROCK1); GSK461364 (GSK461364A; target=PLK1); GW843682X (GW843682; target=PLK1); HG-5-113-01 (target=LOK); HG-5-88-01 (target=EGFR); HG-6-64-01 (KIN001-206; target=ABL1); Neratinib (HKI-272; target=erbB-2); JW-7-24-1 (target=LCK); Dasatinib (BMS-354825; Sprycel; target=ABL1); Tozasertib (VX680; MK-0457; target=Aurora kinase A); GNF2 (target=ABL1); Imatinib (Gleevec; Glivec; CGP-57148B; STI-571; target=ABL1); NVP-TAE684 (TAE-684; target=ALK); CGP60474 (MLS000911536; SMR000463552; target=CDK1); PD173074 (target=FGFR1); Crizotinib (PF02341066; target=c-Met); BMS345541 (target=IKKB); LY2090314; KIN001-042 (target=GSK-3 beta); KIN001-043 (target=GSK-3 beta); Saracatinib (AZD0530; target=Src); KIN001-055 (target=JAK3); AS601245 (JNK Inhibitor V; target=JNK3); Sigma A6730KIN001-102; AKT inhibitor VIII; Akt1/2 kinase inhibitor (target=Akt-1); SB 239063 (target=MK14); AC220 (target=FLT3); WH-4-023 (target=LCK); R406 (target=SYK); BI-2536 (NPK33-1-98-1; target=PLK1); Motesanib (AMG706; target=VGFR1); KIN001-127 (target=ITK); A443654 (target=Akt-1); SB590885 (target=BRAF); Pictilisib (Pictrelisib; GDC-0941; RG-7321; target=PIK3CA); PD184352 (CI-1040; target=MP2K1); PLX-4720 (target=BRAF); AZ-628 (target=BRAF); Lapatinib (GW-572016; Tykerb; target=EGFR); Sirolimus (Rapamycin; target=mTOR); ZSTK474 (target=PIK3CA); AS605240 (target=PIK3CG); BX-912 (target=PDK1); Selumetinib (AZD6244; Arrayl42886; target=MP2K1); MK2206 (target=Akt-1); CG-930 (JNK930; target=JNK1); AZD-6482 (KIN001-193; target=PIK3CB); TAK-715 (target=MK14); NU7441 (KU 57788; target=DNA-PK); GSK1070916 (KIN001-216; target=Aurora kinase B); OSI-027; WYE-125132 (target=mTOR); KIN001-220 (Genentech 10; target=Aurora kinase A); MLN8054 (target=Aurora kinase A); Barasertib (AZD1152-HQPA; target=Aurora kinase B); Vemurafenib (PLX4032; RG7204; R7204; R05185426; target=BRAF); Enzastaurin (LY317615; target=KPCB); NPK76-II-72-1 (target=PLK3); Palbociclib (PD0332991; target=CDK4); PF562271 (KIN001-205; target=FAK); PHA-793887 (target=CDK2); KU55933 (target=ATM); QL-X-138 (target=BTK); QL-XI-92 (target=DDR1); QL-XII-47 (target=BTK); THZ-2-98-01 (target=IRAK1); Torin1 (target=mTOR); Torin2 (target=mTOR); KIN001-244 (target=PDK1); WZ-4-145 (target=CSF1R); WZ-7043 (target=CSF1R); WZ3105 (target=CLK2); WZ4002 (target=EGFR); XMD11-50 (LRRK2-in-1; target=LRRK2); XMD11-85h (target=BRSK2); XMD13-2 (target=RIPK1); XMD14-99 (target=EPHB3); XMD15-27 (target=CAMK2B); XMD16-144 (target=Aurora kinase A); JWE-035 (target=Aurora kinase A); XMD8-85 (target=ERK5); XMD8-92 (target=ERK5); ZG-10 (target=JNK1); ZM-447439 (target=Aurora kinase A); Erlotinib (OSI-774; target=EGFR); Gefitinib (ZD1839; Iressa; target=EGFR); Nilotinib (AMN-107; target=ABL1); JNK-9L (KIN001-204; target=JNK1); PD0325901 (PD-325901; target=MP2K1); MPS-1-IN-1 (HG-5-125-01); XMD-12; YM 201636 (Kin001-170; target=FYV1); FR180204 (FR 180204; KIN001-230; target=ERK-1); TWS119 (target=GSK-3 beta); PF477736 (target=CHK1); Kin237 (Kin001-237; c-Met/Ron dual kinase inhibitor; target=c-Met); Pazopanib (GW786034; Votrient); LDN-193189 (DM 3189; target=ACVR1); PF431396 (target=FAK); Celastrol (target=PSB5); Amuvatinib (MP470; target=PGFRA); SU11274 (PKI-SU11274; target=c-Met); Canertinib (CI-1033; PD-183805; target=EGFR); SB525334 (target=TGFR1); NVP-AEW541 (AEW541; target=IGF1R); SGX523 (target=c-Met); MGCD265 (target=c-Met); PHA-665752 (target=c-Met); PI103 (target=PIK3CA); Dovitinib (TKI_258; TKI258; target=FLT3); GSK 690693 (target=Akt-1); Ibrutinib (PCI-32765; target=BTK); Masitinib (AB1010; target=c-Kit); Tivantinib (ARQ197; target=c-Met); SNS-032 (BMS-387032; target=CDK9); Afatinib (BIBW-2992; target=erbB-2); GSK1904529A (target=IGF1R); Linsitinib (OSI 906; target=IGF1R); TPCA-1 (target=IKKB); BMS509744 (BMS-509744; target=ITK); Ruxolitinib; AZD-1480 (target=JAK2); Momelotinib (CYT387; target=JAK1); Fedratinib (SAR 302503; SAR-302503; SAR302503; TG 101348; Tg-101348; TG101348; target=JAK2); Trametinib (GSK-1120212; GSK1120212; GSK1120212B; JTP-74057; target=MP2K1); BMS 777607 (target=c-Met); Olaparib (AZD2281; KU-0059436; target=PARP-1); Veliparib (ABT-888; target=PARP-1); Omipalisib (GSK2126458; GSK2126458A; target=PIK3CA); Buparlisib (BKM120; NVP-BKM120; target=PIK3CA); XL147 (SAR245408; target=PIK3CA); Y39983 (target=ROCK1); Ponatinib (AP24534; target=ABL1); Nintedanib (BIBF-1120; Vargatef; target=VGFR1); MK 1775 (target=WEE1hu); KIN001-266 (target=M3K8); AT7867 (target=Akt-1); KU-60019 (target=ATM); JNJ38877605 (target=c-Met); Foretinib (XL880; GSK1363089; target=c-Met); AZD 5438 (KIN001-239; target=CDK2); Pelitinib (EKB-569; target=EGFR); SB 216763 (target=GSK-3 beta); Luminespib (NVP-AUY922; target=HS90A); SP600125 (target=JNK1); BIX 02189 (target=MP2K5); AZD8330 (ARRY-424704; ARRY-704; target=MP2K1); PF04217903 (target=c-Met); BAY61-3606 (target=SYK); SB 203580 (RWJ 64809; PB 203580; target=MK14); VX-745 (target=MK14); Doramapimod (BIRB 796; target=MK14); JNJ 26854165 (target=p53); TGX221 (target=PIK3CB); GSK1059615 (target=PIK3CA); PI3K-IN-1 (target=mTOR); A 769662 (target=AMPK-alpha1); Sunitinib (Sutent; SU11248); Y-27632 (target=ROCK1); Brivanib (BMS-540215; target=VGFR1); OSI-930 (target=c-Kit); ABT-737 (target=BCL2); CHIR-99021 (CT99021; KIN001-157; target=GSK-3 beta); GDC-0879 (target=BRAF); Linifanib (ABT-869; AL-39324; target=FLT3); BGJ398 (KIN001-271; NVP-BGJ398; target=FGFR1); Rigosertib (ON-01910; target=PLK1); CC-401 (target=JNK1); Chelerythrine (target=KPCB); Ki20227 (target=CSF1R); BX795 (target=TBK1); Bosutinib (SKI-606; target=Src); PIK-93 (target=PIK3CG); HMN-214 (target=PLK1); KW2449 (KW-2449; target=FLT3); Kin236 (Tie2 kinase inhibitor; target=TIE2); Cabozantinib (XL-184; BMS-907351; target=VEGFR-2); KIN001-269 (target=CSF1R); KIN001-270 (target=CDK9); KIN001-260 (IKK-2 inhibitor VIII; Bayer IKKb inhibitor; target=IKKB); Vandetanib (ZD6474; Zactima; Caprelsa; target=VEGFR-2); PF 573228 (target=FAK); NVP-BHG712 (KIN001-265; target=EPHB4); CH5424802 (target=ALK); D 4476 (target=TGFR1); A66 (target=PIK3CA); CAL-101 (target=PIK3CD); INK-128 (MLN0128; target=mTOR); RAF 265 (CHIR-265; target=BRAF); NVP-TAE226 (target=FAK); or JNK-IN-5A (TCS JNK 5a; KIN001-188; target=MK09).

In certain embodiments, the test agent is a clustered regularly interspaced short palindromic repeats (CRISPR) agent.

In one embodiment, the test agent is identified to enhance CD8⁺ T cell killing of the cells expressing the model antigen peptide.

In another embodiment, the test agent is identified to inhibit CD8⁺ T cell killing of the cells expressing the model antigen peptide.

An additional aspect of the current disclosure provides a cell mixture that includes (A) a first population of cells harboring a nucleotide sequence encoding for a model antigen peptide, operably linked to nucleotide sequence encoding for a first reporter peptide; and (B) a second population of cells harboring a nucleotide sequence encoding for a second reporter peptide.

In certain embodiments, the first population of cells is an ovarian cancer cell population.

In one embodiment, the (1) first population of cells harboring a nucleotide sequence encoding for a model antigen peptide, operably linked to nucleotide sequence encoding for a first reporter peptide and the (2) second population of cells harboring a nucleotide sequence encoding for a second reporter peptide, are derived from the same source cell line, optionally where the source cell line is a carcinoma cell line, optionally an ovarian carcinoma cell line, optionally ID8 cells.

In certain embodiments, the cell mixture further includes a third population of cells that is a CD8⁺ T cell population, optionally where the third population of cells that is a CD8⁺ T cell population is present in at least about a 2:10 proportion to the first population of cells harboring the nucleotide sequence encoding for the model antigen peptide, optionally where the third population of cells that is a CD8⁺ T cell population present in about a 3:10 to about a 10:1 proportion to the first population of cells harboring the nucleotide sequence encoding for the model antigen peptide, optionally where the third population of cells that is a CD8⁺ T cell population present in about a 1:1 to about a 2:1 proportion to the first population of cells harboring the nucleotide sequence encoding for the model antigen peptide. In a related embodiment, the third population of cells that is a CD8⁺ T cell population is an OT-I T cell receptor transgenic cell population.

In one embodiment, the cell mixture is present in an array, optionally in a 96 well plate format.

In another embodiment, the cell mixture includes an about 1:1 proportion of (1) the first population of cells harboring a nucleotide sequence encoding for a model antigen peptide, operably linked to nucleotide sequence encoding for a first reporter peptide and (2) the second population of cells harboring a nucleotide sequence encoding for a second reporter peptide.

In one embodiment, the first population of cells is an immortalized cell line.

In another embodiment, the first reporter peptide is a luciferase peptide, optionally firefly luciferase.

In certain embodiments, the second reporter peptide is a luciferase peptide distinct from the first reporter peptide. Optionally the second reporter peptide is renilla luciferase.

In another aspect, the instant disclosure also provides method for enhancing CD8⁺ T cell killing of target cells in a subject that includes administering a pharmaceutical composition comprising an EGFR inhibitor and a pharmaceutically acceptable carrier to the subject in an amount sufficient to enhance CD8⁺ T cell killing of target cells in the subject.

In one embodiment, the target cells are ovarian cancer cells, lung cancer cells, colorectal cancer cells, glioblastoma cells, breast cancer cells, prostate cancer cells, renal cancer cells, melanoma and/or pancreatic cancer cells.

In certain embodiments, the subject is human.

In other embodiments, the subject is murine.

In one embodiment, the target cells are cells of a cancer cell line, optionally an ovarian cancer cell line, optionally ID8 cells.

In certain embodiments, the EGFR inhibitor is erlotinib, gefitinib, afatinib and/or osimertinib.

In an additional aspect, the instant disclosure provides a method for inhibiting CD8⁺ T cell killing of target cells in a subject, the method involving administering a pharmaceutical composition comprising a j anus kinase 2 (JAK2) inhibitor and a pharmaceutically acceptable carrier to the subject in an amount sufficient to inhibit CD8⁺ T cell killing of target cells in the subject.

In one embodiment, the JAK2 inhibitor is AZD-1480, Pacritinib, Gandotinib, XL019, BMS-911543, AZ 960, Fedratinib, NVP-BSK805 2HCl or CEP-33779.

An additional aspect of the invention provides a method for treating or preventing a neoplasia in a subject that involves administering a pharmaceutical composition to a subject that includes (i) an EGFR inhibitor; (ii) an anti-PD-1 agent, an anti-CTLA agent, an anti-KIR agent, an anti-TIGIT agent, an anti-TIM-3 agent, an anti-LAG-3 agent, a 4-1BB agonist, an ICOS agonist, a GITR agonist or a CD28 agonist; and (iii) a pharmaceutically acceptable carrier in an amount sufficient to treat or prevent neoplasia in the subject.

In certain embodiments, the neoplasia is an ovarian cancer, a lung cancer, a colorectal cancer, a glioblastoma, a breast cancer, a prostate cancer, a renal cancer, a melanoma or a pancreatic cancer.

In some embodiments, the anti-PD-1 agent, anti-CTLA agent, anti-KIR agent, anti-TIGIT agent, anti-TIM-3 agent, anti-LAG-3 agent, 4-1BB agonist, ICOS agonist, GITR agonist or CD28 agonist is an antibody.

In one embodiment, the EGFR inhibitor is erlotinib, gefitinib, afatinib or osimertinib.

A further aspect of the invention provides a pharmaceutical composition for the treatment of neoplasia that includes (i) an EGFR inhibitor; (ii) an anti-PD-1 agent, an anti-CTLA agent, an anti-KIR agent, an anti-TIGIT agent, an anti-TIM-3 agent, an anti-LAG-3 agent, a 4-1BB agonist, an ICOS agonist, a GITR agonist or a CD28 agonist; and (iii) a pharmaceutically acceptable carrier.

Another aspect of the disclosure provides a method for enhancing CD8⁺ T cell killing of target cells in a subject that involves administering a pharmaceutical composition that includes a Noc4I inhibitor, a Prpf19 inhibitor, a Prmt5 inhibitor, a Fbxw7 inhibitor, an Eif3a inhibitor, a Cd274 inhibitor, a Mta2 inhibitor, a Nat10 inhibitor and/or a Map3k7 inhibitor and a pharmaceutically acceptable carrier to a subject in an amount sufficient to enhance CD8⁺ T cell killing of target cells in the subject.

In certain embodiments, the target cells are ovarian cancer cells, lung cancer cells, colorectal cancer cells, glioblastoma cells, breast cancer cells, prostate cancer cells, renal cancer cells, melanoma and/or pancreatic cancer cells.

Optionally, the subject is human. In other embodiments, the subject is murine.

In some embodiments, the target cells are cells of a cancer cell line, optionally an ovarian cancer cell line, optionally ID8 cells.

In one embodiment, the Noc4I inhibitor, Prpf19 inhibitor, Prmt5 inhibitor, Fbxw7 inhibitor, Eif3a inhibitor, Cd274 inhibitor, Mta2 inhibitor, Nat10 inhibitor and/or Map3k7 inhibitor is a CRISPR agent and/or an inhibitory nucleic acid.

An additional aspect of the disclosure provides a method for inhibiting CD8⁺ T cell killing of target cells in a subject that involves administering a pharmaceutical composition that includes a H2-K1 inhibitor, a Hdac8 inhibitor, a Tap1 inhibitor, an Ep300 inhibitor, a Tap2 inhibitor, a Cbx5 inhibitor, a B2m inhibitor, a Brwd1 inhibitor, a Cbx3 inhibitor and/or a Chrac1 inhibitor and a pharmaceutically acceptable carrier to a subject in an amount sufficient to inhibit CD8⁺ T cell killing of target cells in the subject.

In one embodiment, the H2-K1 inhibitor, Hdac8 inhibitor, Tap1 inhibitor, Ep300 inhibitor, Tap2 inhibitor, Cbx5 inhibitor, B2m inhibitor, Brwd1 inhibitor, Cbx3 inhibitor and/or Chrac1 inhibitor is a CRISPR agent and/or an inhibitory nucleic acid.

A further aspect of the disclosure provides a method for treating or preventing a neoplasia in a subject that involves administering a pharmaceutical composition to the subject that includes (i) a Noc4I inhibitor, a Prpf19 inhibitor, a Prmt5 inhibitor, a Fbxw7 inhibitor, an Eif3a inhibitor, a Cd274 inhibitor, a Mta2 inhibitor, a Nat10 inhibitor and/or a Map3k7 inhibitor; (ii) an anti-PD-1 agent, an anti-CTLA agent, an anti-KIR agent, an anti-TIGIT agent, an anti-TIM-3 agent, an anti-LAG-3 agent, a 4-1BB agonist, an ICOS agonist, a GITR agonist or a CD28 agonist; and (iii) a pharmaceutically acceptable carrier, in an amount sufficient to treat or prevent the neoplasia in the subject.

In certain embodiments, the anti-PD-1 agent, anti-CTLA agent, anti-KIR agent, anti-TIGIT agent, anti-TIM-3 agent, anti-LAG-3 agent, 4-1BB agonist, ICOS agonist, GITR agonist or CD28 agonist is an antibody.

In some embodiments, the Noc4I inhibitor, Prpf19 inhibitor, Prmt5 inhibitor, Fbxw7 inhibitor, Eif3a inhibitor, Cd274 inhibitor, Mta2 inhibitor, Nat10 inhibitor and/or Map3k7 inhibitor is a CRISPR agent and/or an inhibitory nucleic acid.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 5%, 5%, 4%, 3%, 2%, 1%, 0.5%, 0.1%, 0.05%, or 0.01% of the stated value. Unless otherwise clear from context, all numerical values provided herein are modified by the term “about.”

By “agent” is meant any small compound (e.g., small molecule), antibody, nucleic acid molecule, or polypeptide, or fragments thereof.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein the term “CD8⁺ T cells” has its general meaning in the art and refers to a subset of T cells which express CD8 on their surface, are MHC class I-restricted, and function as cytotoxic T cells. “CD8” molecules are differentiation antigens found on dendritic cells, on thymocytes and on cytotoxic and suppressor T-lymphocytes. CD8 antigens are members of the immunoglobulin supergene family and are associative recognition elements in major histocompatibility complex class I-restricted interactions.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CML, T cell CML), and chronic lymphocytic leukemia (CLL) (e.g., B cell CLL, T cell CLL)); lymphoma such as Hodgkin lymphoma (HL) (e.g., B cell HL, T cell HL) and non-Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa-associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T cell NHL such as precursor T-lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis-like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

“Detect” refers to identifying the presence, absence, or amount of the agent (e.g., a nucleic acid molecule, for example deoxyribonucleic acid (DNA) or ribonucleic acid (RNA)) to be detected.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By “fragment” is meant a portion, e.g., a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full-length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The term “in vitro” as used herein refers to events that occur in an artificial environment, e.g., in a test tube or reaction vessel, in cell culture, etc., rather than within a multi-cellular organism.

As used herein “in vivo” refers to events that occur within a multi-cellular organism, such as a human and a non-human animal. In the context of cell-based systems, the term may be used to refer to events that occur within a living cell (as opposed to, for example, in vitro systems).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

As used herein, a “model antigen peptide” refers to an antigen to which a CD8⁺ T cell is capable of forming a cytotoxic response. In certain embodiments, a “model antigen peptide” is a peptide to which a CD8⁺ T cell has been designed to respond (e.g., designed via transgenic methods to respond to a specific model antigen). An exemplary model antigen peptide is chicken ovalbumin, which is a T cell dependent antigen often used as a model protein for studying antigen-specific immune responses in mice and/or mouse cell lines.

As used herein, “neoplasia” means a disease state of a human or an animal in which there are cells and/or tissues which proliferate abnormally. Neoplastic conditions include, but are not limited to, cancers, sarcomas, tumors, leukemias, lymphomas, and the like. A neoplastic condition refers to the disease state associated with the neoplasia. Hepatocellular carcinoma, colon cancer (e.g., colorectal cancer), lung cancer and ovarian cancer are examples (non-limiting) of a neoplastic condition. A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell. Examples of cancer include but are not limited to hepatic carcinoma, colon cancer, colorectal cancer, breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, esophageal cancer, gastrointestinal cancer, glioblastoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e., a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

Unless specifically stated or obvious from context, as used herein; the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating mated al; involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil; safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions, and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility, factors that are statistically correlated with increased risk of development of the relevant disease, disorder, and/or condition.

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 depicts an illustrated representation of the cells and associated molecular components designed and used within the OT-I CTL (Ovalbumin-specific CD8⁺ T cell receptor transgenic line OT-I cytotoxic T lymphocyte) screen of the instant disclosure.

FIG. 2A to FIG. 2C depict an illustrated representation of OT-I CTL screen design. FIG. 2A shows that ID8-Cas9 serous ovarian carcinoma cell line (“clone A10”) cells were transduced with pLVX vector expressing either firefly luciferase fused to model antigen peptide (here, ovalbumin) or to renilla luciferase with no antigen. Clonal cell lines were generated using G418 selection for Neo cassette expression and limiting dilution. FIG. 2B shows that 10,000 ID8-lucOS and 10,000 ID8-rluc were co-plated into wells of 96-well tissue culture plates. OT-I TCR transgenic CD8⁺ T cells were then plated on top of ID8 cells, and these transgenic CD8⁺ T cells were observed to selectively kill ID8-lucOS in an antigen-dependent manner, while sparing ID8-rluc. Total volume/well was 2004, and assayed cells were incubated for 48 hr at 37° C. and 5% 02 prior to analysis by dual luciferase assay. FIG. 2C shows performance of the OT-I assay as a high-throughput screen to evaluate compounds for immunomodulatory effects upon antigen-specific tumor cell killing by cytotoxic T lymphocytes (CTLs). Inclusion of ID8-lucOS and ID8-rluc provided in-plate normalization controls, which allowed for identification of non-specific growth inhibition or induction of apoptosis by screen compounds, versus identification of modulation by screen compounds of antigen-specific tumor cell killing by cytotoxic T lymphocytes. Such high-throughput screening can be performed under standard cell growth conditions (e.g., at 37° C. and 5% O₂) or can be performed under a number of other conditions, including, e.g., in the presence of hypoxia, hydrogen peroxide (H₂O₂), TGF-β/IL-10, T regulatory cells (Tregs), myeloid-derived suppressor cells (MDSCs), in the absence of L-arginine and/or L-cysteine, etc.

FIG. 3A to FIG. 3D depict validation results for the OT-I assay of the disclosure. FIG. 3A shows a histogram depicting the dose-responsiveness of firefly luciferase levels (expressed by ID-8 ovarian cancer cells also expressing ovalbumin as a model antigen peptide), which declined with administration of increasing numbers of OT-I CD8⁺ T cells. In such experiments, 10,000 ID8-lucOS (expressing ovalbumin) and 10,000 ID8-rluc (not expressing ovalbumin) were plated in 96-well plates with varying levels of OT-I CD8⁺ T cells to assess antigen-specific tumor cell killing. A significant decrease in firefly luciferase expression was observed with increasing Effector:Target ratios, whereas renilla luciferase was unaffected. FIG. 3B and FIG. 3C show that similar levels of dose-response to OT-I CD8⁺ T cells were observed via normalization of firefly luciferase levels to renilla luciferase levels (FIG. 3B) or by calculating % survival of target ID8-lucOS cells (FIG. 3C). Each showed significant antigen-specific tumor cell killing with E:T (Effector to target cell) ratios as low as 0.31 and approximately 50% killing at E:T ratio of 1. FIG. 3D shows that administration of cyclosporin A, which is an inhibitor of calcineurin and a well-characterized inhibitor of CD8⁺ T cell effector function, was capable of reversing the impact of adding CD8⁺ T cells to the target ID8-lucOS cell-containing population. Cyclosporin A was therefore used as a control compound to validate assay performance. Experiments were performed at least twice with six replicate wells per condition. Data for bar graphs were calculated using unpaired Student's t-test with p<0.05 as *, p<0.01 as **, and p<0.001 as *** and presented as mean with SD.

FIG. 4A-FIG. 4C shows that compounds with general inhibitory effects on cell growth effect both ID8-Cas9-lucOS (with OVA) and ID8-Cas9-rluc (no OVA) to equal degrees. ˜⅓ of screen compounds (shaded red) cause this phenotype. Specifically, shown are inhibitory effects of the 203 compound library upon both types of ID8 tumor cells employed, in the absence of OT-I T cells. DMSO control wells were identified as results that should be consistent across all assays, because the DMSO shouldn't affect the ID8 tumor cell viability. Accordingly, normalizing raw luciferase values relative to the DMSO average was predicted to provide a distribution with most compounds around 1 (that don't affect growth) and some fraction above (that augment growth) or below 1 (that inhibit growth). Many compounds were thereby identified as non-specifically inhibiting ID8 cell growth, validating the need for inclusion of rluc-expressing ID8 cells as control cells within the OT-I assays of the current disclosure.

FIG. 5 shows the distribution of effects observed for the 203 test compounds initially administered in the OT-I screen of the disclosure. Normalized firefly/renilla luciferase ratios relative to DMSO-only control wells are shown for each test compound. Compounds that inhibited OT-I T cell killing exhibited ratios <1 (JAK2 inhibitor, CDK9 inhibitor, PLK1 inhibitor), inert compounds exhibited ratios ˜1, and compounds that augmented T cell killing displayed ratios >1 (e.g., EGFR inhibito, GSK-3β inhibitor). Plates were screened in duplicate, and compounds were considered “hits” only if they scored in both plates.

FIG. 6A to FIG. 6E show histograms that demonstrate validation of screen results across four different test compounds. FIG. 6A shows that control compound cyclosporin A exhibited a predicted, dose-responsive inhibition of OT-I T cell-mediated killing (increasing amounts of cyclosporin A maintained firefly luciferase levels by blocking CD8⁺ T cell-mediated killing of ovalbumin-expressing cells). FIG. 6B shows that AZD 1480 (a JAK2 inhibitor), which was the top hit of the 203 test compound screen for inhibition of T cell-mediated killing, performed similarly to cyclosporin A, which thereby supported the assessment from the larger compound screen that AZD 1480 could also disrupt CD8⁺ T cell-mediated killing of ovalbumin-expressing cells, as was demonstrated for AZD 1480 across a broader dose range (thereby verifying the similar dose-responsiveness of the observed effect). FIG. 6C to FIG. 6E show that erlotinib (an EGFR inhibitor, FIG. 6C), which was identified as the top hit of the 203 test compound screen for augmenting T cell-mediated killing, as well as two other EGFR inhibitors (gefitinib—FIG. 6D, afatinib—FIG. 6E) impacted CD8⁺ T cell-mediated killing in a dose-responsive manner, at least at higher test compound concentrations (increasing levels of the EGFR inhibitors increased T cell-mediated killing in the screening assay). Inhibition of EGFR with any of these test compounds therefore augmented antigen-specific CD8⁺ T cell-mediated killing. Data were again presented as raw firefly luciferase (OVA-expressing ID8) values for two different effector:target ratios (left-hand histograms), relative firefly luciferase values that account for drug impacts on ID8 survival irrespective of CD8⁺ T cell-mediated killing (middle tables), and % survival of ID8-lucOS target cells (right-hand histograms). Experiments were each conducted at least twice, and similar results were observed across tested replicates of four wells per condition (per test compound administered). Data for histograms were calculated using unpaired Student's t-test with p<0.05 as *, p<0.01 as **, and p<0.001 as *** and presented as mean with SD.

FIG. 7A, FIG. 7B, FIG. 7C, FIG. 7D, and FIG. 7E show that EGFR inhibition enhanced T cell killing via what appeared to be a tumor cell intrinsic mechanism. In FIG. 7A, ELISA revealed that EGFR inhibitor (erlotinib, gefitinib and afatanib were all tested, in parallel with the AZD 1480 compound, which was newly identified as an inhibitor of T cell-mediated killing of target cells, and which dramatically decreased T cell IFN-γ production in a dose-dependent manner) and dose did not affect secretion of IFN-γ by OT-I CD8⁺ T cells, in the same assay where compound treatment produced enhanced killing of target tumor cells. FIG. 7B-FIG. 7E are graphs that show inhibition of EGFR with three different compounds of varying chemotypes, or sgRNA targeting EGFR, increases basal and IFN-γ-induced surface expression of MHC class-I by target tumor cells. For example, FIG. 7B shows that ID8 MHC Class I expression levels were significantly elevated in the presence of EGFR inhibitors (erlotinib, gefitinib and afatanib) relative to control (DMSO) treatments, and that such expression levels were significantly elevated under all conditions in the presence of 4 pg/mL IFN-γ, as assessed by detection of H2-Kb MFI values. Experiments were conducted at least twice with similar results and in replicates of four wells per condition. Data for bar graphs were calculated using unpaired Student's t-test with p<0.05 as *, p<0.01 as **, and p<0.001 as *** and presented as Mean with SD.

FIG. 8A to FIG. 8G show that EGFR inhibition enhanced efficacy of PD-1 blockade. FIG. 8A and FIG. 8B show that mice receiving combination treatment of anti-PD-1 and the EGFR inhibitor, afatinib, exhibited significantly reduced tumor burden on day 12. FIG. 8C shows that mice receiving combination treatment of anti-PD-1 and the EGFR inhibitor, afatinib, exhibited significantly inhibited tumor growth kinetics. FIG. 8D shows that mice receiving combination treatment of anti-PD-1 and the EGFR inhibitor, afatinib, exhibited significantly improved survival relative to other treatments. For each of the experiments, C57BL/6J mice were challenged subcutaneously with 500,000 MC38 colon cancer cells on their flanks and “enrolled” on-study when tumors reached 50 mm³. Mice were treated with aPD-1 on days 5, 8, and 12 and afatinib on days 6, 7, 8, 9, and 10 (where indicated). For survival curves: vehicle vs. afatinib n.s.; vehicle vs. aPD-1 p=0.013; vehicle vs. combo p=0.003; afatinib vs. aPD-1 p=0.069; afatinib vs. combo p=0.017; aPD-1 vs. combo n.s; vehicle vs. combo CD8 depleted n.s.; afatinib vs combo CD8 depleted n.s.; aPD-1 vs. combo CD8 depleted p=0.071; combo vs. combo CD8 depleted p=0.028.

FIG. 8E and FIG. 8F are graphs showing the response to afatinib and pembrolizumab combination therapy in retrospective cohort of 41 Taiwanese patients with SCCHN. Data presented as pre- and post-treatment scans of selected responders (FIG. 8E), swimmer's plot of treatment and progression (FIG. 8F), and % change in tumor volumes from baseline (FIG. 8G). Flank tumor growth curves were analyzed using two-way ANOVA, bar graphs were analyzed using unpaired Student's t-test, and survival experiments used the log-rank Mantel-Cox test for survival analysis, all indicated with *p<0.05; **p<0.01; ***p<0.001.

FIG. 9A to FIG. 9H is a series of graphs illustrating that the CRISPR/Cas9 screen identifies sgRNAs targeting EGFR as sensitizing tumor cells to T cell killing. Specifically, shown are the results of screening performed using input sgRNAs as screening agents. ID8-lucOS cells alone or co-cultured at E:T of 1:1 with OT-I T cells were incubated for 72 hours, after which genomic DNA was isolated and sgRNA sequences were deconvoluted by NGS. FIG. 9A shows the distribution of assayed sgRNA representation levels in live versus dead cells in the absence of OT-I CD8⁺ T cells. FIG. 9B shows representation data for the ten sgRNAs that exhibited the greatest enrichment in live cells (versus dead cells). FIG. 9C shows representation data for the ten sgRNAs that exhibited the greatest depletion in live cells (versus dead cells). FIG. 9D shows the distribution of assayed sgRNA representation levels in live versus dead cells in the presence of OT-I CD8⁺ T cells. sgRNA targeting MHC genes were enriched in +OT-I cultures, while sgRNAs targeting PD-L1 and EGFR were depleted. CRISPR score is defined as the average log 2 fold-change in abundance of sgRNAs for each gene (10sgRNA/gene) relative to sgRNA library plasmid pool. Specifically, FIG. 9E shows representation data for the ten sgRNAs that exhibited the greatest enrichment in live cells (versus dead cells) when assayed in the presence of OT-I CD8⁺ T cells. FIG. 9F shows representation data for the ten sgRNAs that exhibited the greatest depletion in live cells (versus dead cells) when assayed in the presence of OT-I CD8⁺ T cells. FIG. 9G shows a summary of live vs. dead cell values across all sgRNAs tested. FIG. 9H shows similar summary values for bins of EGFR sgRNAs tested/identified, H2-K1 sgRNAs assayed/identified, as compared to control sgRNAs assayed/identified. Notably, consistent with the results of FIG. 9F, EGFR sgRNAs showed a bias towards dead cells rather than live cells. Meanwhile, consistent with the results of FIG. 9E, H2-K1 sgRNAs showed a bias in the opposite direction, towards live cells as opposed to dead cells, as compared to sgRNA controls.

FIG. 10A and FIG. 10B show that Cas9 was active in ID8 cells of the instant assays, and that these cells responded to IFN-γ by upregulating PD-L1, which could also be successfully prevented by transducing the cells with sgRNAs targeting the PD-L1 gene. Specifically, ID8-Cas9 cells transduced with sgRNAs targeting B2m abrogate surface expression of MHC class-I. ID8-Cas9 cells transduced with sgRNAs targeting PD-L1 reduce surface expression of PD-L1 when induced with physiological levels of recombinant IFN-γ.

FIG. 11A-FIG. 11D are a series of bar graphs and charts showing selective GSK-30 and pan-GSK-3 inhibitors are only mildly immunomodulatory validation of initial screen result. Osimertinib induces modestly enhanced target cell killing. FIG. 11A shows results with 6-bromoindirubin GSK-3B inhibitor. FIG. 11B shows results with indirubicin GSK-3 inhibitor. FIG. 11C shows results with tideglusib GSK-3B inhibitor. FIG. 11D shows results with osimertinib.

FIG. 12A-FIG. 12E are a series of bar graphs showing that EGFR TKI augments tumor killing in KP cell line. Shown is a repeat of OT-I CTL assay with a Kras^{G12D}/p53^{-/-} cell line recapitulated the result observed in ID8 ovarian cells: EGFR inhibitors also enhance T cell-mediated tumor cell lysis. FIG. 12A shows results with KP-Cas9 puro. FIG. 12B shows results with erlotinib. FIG. 12C shows results with gefitinib. FIG. 12D shows results with afatinb. FIG. 12E shows results with cyclosporine A.

FIG. 13A and FIG. 13B show CRISPR/Cas9 engineered KO of EGFR sensitizes tumor cells to CTL-mediated killing. EGFR was knocked out in ID8-Cas9-lucOS cells using top-scoring EGFR-targeting sgRNA from CRISPR/Cas9 pooled screen. KO of EGFR significantly sensitized target cells to CTL killing across a range of E:T ratios. FIG. 13A is a photograph of an immunoblot. FIG. 13B is a bar chart showing Effector:Target ratio and % survival.

FIG. 14 is a series of line graphs demonstrating individual tumors and tolerability. Spider plots of individual tumor progression in different treatment groups (15-16 mice/group). Tracking of body weight changes clearly shows acceptable tolerability of aPD-1+afatinib combination.

FIG. 15 is a series of charts showing the clinical annotation of a retrospective analysis of afatinib+pembrolizumab in squamous cell carcinoma of the head and neck (SCCHN). Shown are clinical characteristics of 41 Taiwanese patients receiving combination afatinib and pembrolizumab anti-PD-1, response to therapy, and toxicity information.

### DETAILED DESCRIPTION OF THE INVENTION

The present invention is directed, at least in part, to development of a high-throughput screening assay capable of identifying immunomodulatory therapeutic agents. In certain aspects, cell mixtures specifically designed for use in such screening assays are provided. Other aspects of the disclosure provide methods for therapeutic use of the immunomodulatory properties of agents identified by the instant screening process, including use of EGFR inhibitory agents possessing the ability to enhance CD8⁺ cytotoxic T lymphocyte-mediated killing of target cells that display MHC-1 antigens.

With the FDA approval of immune checkpoint blocking antibodies, initially targeting CTLA-4 in melanoma (Hodi et al., 2010 N Engl J Med, 363:711-23), and more recently and rapidly for PD-1/PD-L1 in melanoma (Postow et al., 2015 N Engl J Med, 372:2006-17), NSCLC (Gettinger et al., 2015 J Clin Oncol, 33:2004-12), head and neck cancer (Ferris et al., 2016 N Engl J Med, 375:1856-67), and others (Balar et al., 2017 Lancet Lond Engl, 389:67; Motzer et al., 2015 N Engl J Med, 373:1803-13; Younes et al., 2016 Lancet Oncol, 17:1283-94), the field of medical oncology has experienced a paradigm shift in treatment modalities. Combination CTLA-4 and PD-1/PD-L1 blocking antibodies have exhibited synergistic efficacy (Postow et al., 2015 N Engl J Med, 372:2006-17; Larkin et al., 2015 N Engl J Med, 373:23-34). Additionally, there are numerous ongoing trials and pre-clinical development pipelines utilizing antibodies that block one or both of these immune checkpoints in combination with additional checkpoint-blocking antibodies (LAG-3, TIM-3, TIGIT, B7-H3) or agonistic monoclonal antibodies (4-1BB, OX-40, GITR, CD40, ICOS). However, despite all these approaches, not all patients benefit from immunotherapy and, as such, prior to the invention described herein, additional therapeutic strategies to enhance the effects of immunotherapy were needed.

There is increasing interest in combination therapies that leverage existing technologies to increase the immunogenicity of solid tumors and augment immunotherapeutics such as anti-PD-1/PD-L1 that are increasing being viewed as foundational reagents in the medical oncology field. Indeed, radiotherapy (Kwon et al., 2014 Lancet Oncol, 15:700), chemotherapy (Lynch et al., 2012 J Clin Oncol Off J Am Soc Clin Oncol, 30:2046-54; Robert et al., 2011 N Engl J Med, 364:2517-26), and targeted agents such as inhibitors of HDACs (NCT02619253, NCT02437136), BRAF (NCT02818023), and VEGF (NCT00790010) have been or are being tested clinically in combination with immune checkpoint blockade and have been shown to increase response rates.

Combining conventional therapeutics with checkpoint blockade is an attractive strategy, principally given the preexisting pharmacodynamic/pharmacokinetic and toxicology properties of such compounds. Described herein is an assay that is utilized to screen compound libraries in high-throughput for identification of immunomodulatory features. Described herein is the engineering of a target tumor cell line to express firefly luciferase and a model antigen. These target cells were co-cultured with transgenic CD8+ T cells recognizing the model antigen such that modulation of antigen-specific T cell-mediated killing could be assessed by luminescence readout and identify candidate compounds with immunomodulatory properties. The screen identified the epidermal growth factor receptor (EGFR) as a previously unappreciated immune-oncology target whose inhibition dramatically enhances anti-PD-1 immunotherapy.

As immune checkpoint blocking antibodies increasingly become foundational therapies for the treatment of cancer, prior to the invention described herein, there was a pressing need to identify compounds that synergize with checkpoint blockade as the basis of combinatorial treatment regimens. Described herein is the development of a screening assay in which a luciferized tumor cell line expressing a model antigen is co-cultured with a transgenic CD8+ T cell specifically recognizing the model antigen in a H-2^{b}-restricted manner. As described in detail below, the target tumor cell/T cell assay was screened with a small molecule library to identify compounds that inhibit or enhance T cell-mediated killing of tumor cells in an antigen-dependent manner. The EGFR inhibitor, erlotinib, was the top hit that enhanced T cell killing of tumor cells. Subsequent experiments with erlotinib and additional EGFR inhibitors validated the screen result. EGFR inhibitors increase both basal and IFN-γ-induced antigen processing and presentation of MEW class-I, which enhanced recognition and lysis by CD8+ cytotoxic T lymphocytes. The tumor cell line was also transduced to constitutively express Cas9, and a pooled CRISPR screen utilizing the same target tumor cell/T cell assay identified sgRNAs targeting EGFR as sensitizing tumor cells to T cell-mediated killing. As described in detail below, combination of PD-1 blockade with EGFR inhibition showed significant synergistic efficacy in the MC38 syngeneic colon cancer model that was superior to PD-1 blockade or EGFR inhibition alone, further validating EGFR inhibitors as immunomodulatory agents that enhance PD-1 checkpoint blockade. As described herein, this target tumor cell/T cell assay is screened in high-throughput with small molecule libraries and genome-wide CRISPR/Cas9 libraries to identify both compounds and target genes, respectively, that enhance or inhibit T cell recognition and killing of tumor cells.

This screening tool described herein identifies compounds and genes previously not known to affect the immune response to cancer. The identification and validation of EGFR inhibitors as enhancing T cell-mediated killing of tumor cells exemplifies this approach and constitutes the identification of immune checkpoint blockade-enhancing compounds.

### Cytotoxic T Cells

The term “cytotoxic T cell” and its abbreviation “CTL” as used herein may be understood in the broadest sense as any T lymphocyte that is able to induce cell death, in particular in neoplastic cells, cells that are infected, particularly viruses-infected cells, and/or cells in other pathologic conditions. In this context, the terms “cytotoxic T cell”, “CTL”, “cytotoxic TC”, “cytotoxic T lymphocyte”, “T killer cell” “cytolytic T cell” and “killer I cell” may be understood interchangeably. The cytotoxic T cell may be a cytotoxic CD8 T cell. Typically, a CTL in the context of the present invention has at least one T cell receptor (TCR) on its surface that enables the recognition of particular molecular structures presented at surfaces of other cells. Those molecular structures will typically be antigens presented at the surface of the other cell in complex with major histocompatibility complex (MHC) class I, where they can be recognized by the CTL. If the TCR is specific for that antigen, it will bind to said complex of the MHC class I with the antigen and a CTL response occurs, i.e., the other cell is destroyed. Typically, the CTLs used in the context of the present disclosure are mammalian CTLs—in certain embodiments, mouse CTLs are used, so that the CTL response is a mouse CTL response; optionally human CTLs are employed, so that the CTL response is a human CTL response.

### OT-I CTL Cells

OT-I CTL cells of the instant disclosure refer to homozygous mice containing transgenic inserts for mouse Tcra-V2 and Tcrβ-V5 genes. The transgenic T cell receptor was designed to recognize ovalbumin residues 257-264 (SIINFEKL) in the context of H2Kb and used to study the role of peptides in positive selection and the response of CD8⁺ T cells to antigen. Like most TCR transgenics, these mice are somewhat immunodeficient.

### Target Cells

Target cells of the instant disclosure can be any art-recognized cell or cell line that expresses MHC-I and is capable of presenting an antigen to a CTL, thereby inducing targeting of the target cell by the antigen-activated CTL. As recognized by one of ordinary skill in the art, target cells can be derived from many cell lines, including, e.g., various art-recognized cancer cell lines and/or other immortalized cell lines. In certain embodiments, target cells of the disclosure express chicken ovalbumin as a model antigen peptide that is specifically recognized by OT-I TCR transgenic CD8⁺ T cells; however, target cells presenting other antigen peptides are expressly contemplated for use in the methods of the disclosure, with design and use of transgenic CD8⁺ T cells capable of specific recognition of such other antigen peptides also expressly contemplated.

Exemplary target cell lines include the exemplified ID8 ovarian cancer cell line (described below), as well as the CT26 murine colon cancer cell line; the MBT-2 murine bladder cancer cell line; the GL261 murine glioblastoma cell line; the 4T1 (e.g., 4T1-luciferase) and EMT-6 murine mammary carcinoma cell lines; the Colon26 and MC38 murine colon cancer cell lines; the KLN205, Lewis Ling and Madison109 murine lung cancer cell lines; the A20 and E.G7-OVA murine lymphoma cell lines; the B16F10 and CloudmanS91 murine melanoma cell lines; the Pan02 murine pancreatic cancer cell line; and the Renca murine renal cancer cell line, among others.

### ID8 Ovarian Cancer Cell Line

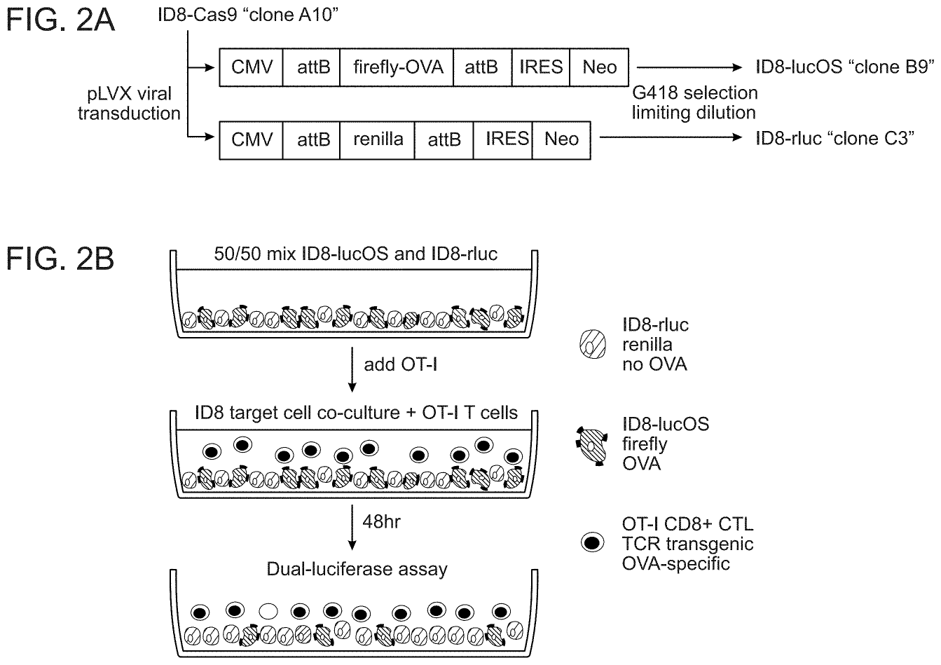

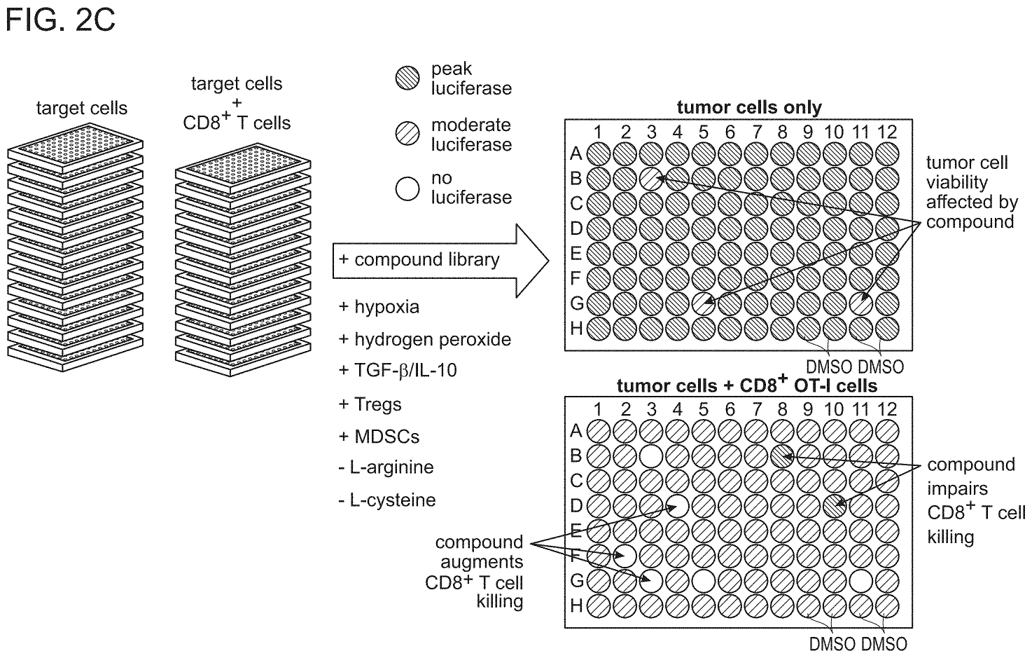

ID8 is a mouse ovarian surface epithelium (MOSE) spontaneously transformed cell line that is physiologically and biologically closely resembling human epithelial ovarian cancer (Roby et al. Carcinogenesis 21: 585-591). In certain aspects of the instant disclosure, ID8 cells can be transformed and/or transduced (optionally virally transduced) with expression cassettes such as those depicted in FIG. 2A, optionally resulting in MHC-I-mediated presentation of a model antigen peptide, such as chicken ovalbumin, at the cell surface.

### Reporter Genes

Reporter genes are used throughout the biological sciences as a means to identify and analyze regulatory elements and/or expression levels of genes. Using recombinant DNA techniques, reporter genes can be fused to other genes and/or to regulatory sequence(s) of interest. The resulting recombinant is then introduced into cells where the expression of the reporter can be detected using various methods, including measurement of the reporter mRNA, measurement of the reporter protein (optionally, presented as a reporter peptide component of a fusion protein), or measurement of the reporter enzymatic activity. Commonly used reporter genes include beta-galactosidase, firefly luciferase, bacterial luciferase, Renilla luciferase, alkaline phosphatase, chloramphenicol acetyltransferase (CAT), green fluorescent protein (GFP) and beta-glucuronidase (GUS).

Many reporter systems utilize luciferase genes. Luciferase refers to a group of enzymes that catalyze the oxidation of various substrates to produce a light emission. Generally, luciferase activity is not found in eukaryotic cells. Thus, it is advantageous for studying promoter activity in mammalian cells. The most popular luciferases for use as reporter genes are the bacterial luciferases, the firefly (Photinus pyralis) luciferase, the Aequorin luciferase and more recently the Renilla luciferase. The different luciferases have different specific requirements and may be used to detect and quantify a variety of substances. For example, one major application for the use of the firefly luciferase is to detect the presence of ATP. The purified jellyfish photoprotein, aequorin, is used to detect and quantify intracellular Ca²⁺. The wild-type luciferase enzyme of the sea pansy Renilla reniform is a monomeric protein with a molecular weight of 36 kDa. This enzyme catalyzes the emission of visible light in the presence of oxygen and the luciferin coelenterazine to produce blue light. The luciferase gene from Renilla has been used to assay gene expression in bacterial (Jubin et al., Biotechniques 24:185-188 (1998)), yeast (Srikantha et al., J. Bacteriol. 178:121-129 (1996)), plant (Mayerhofer et al., Plant J. 7:1031-1038 (1995)), and mammalian cells (Lorenz et al., J. Biolumin. Chemilumin. 11:31-37 (1996)). The cloning, expression and use of wild-type Renilla luciferase are reported in U.S. Pat. Nos. 5,292,658 and 5,418,155.

Firefly luciferase and Renilla luciferase are available commercially (Boehringer Mannheim, Sigma, and Promega). Promega has developed a synthetic Renilla luciferase gene that contains codons optimized for efficient expression in mammalian cells. Literature from Promega indicates that additional features of this modified gene include removal of potentially interfering restriction sites and genetic regulatory sites from the gene (Promega Technical Manual No. 055, revised 6/01). Sequence information related to various plasmids containing the Promega humanized Renilla luciferase gene are deposited with GenBank under accession numbers AF362545-AF362551 .

Other examples of genes and reporter genes optimized for expression in mammalian cells are known in the art. For example, Seed et al. report a method for increasing the expression of eukaryotic and viral genes in eukaryotic cells that involves replacing non-preferred amino acid codons with preferred codons that encode the same amino acid (U.S. Pat. No. 6,114,148; Haas et al., Current Biology 6:315-323 (1996)) (both incorporated herein by reference). Muzyczka et al. (U.S. Pat. No. 6,020,192) and Zolotukhin, et al. (J. Virology 70:4646-4654 (1996)) report green fluorescent proteins optimized for expression in mammalian cells. Sherf et al. report a modified beetle luciferase (U.S. Pat. No. 5,670,356).

### EGFR Inhibitors

As used herein, an “EGFR gene” refers to a nucleic acid that encodes an EGFR gene product, e.g., an EGFR mRNA, an EGFR polypeptide, and the like. As used herein, “EGFR inhibitor” refers to any agent capable of directly or indirectly inhibiting activation of an EGFR. EGFR inhibitors include agents that bind to an EGFR and inhibit its activation. EGFR inhibitors include antibodies that bind to an EGFR and inhibit activation of the EGFR; as well as small molecule tyrosine kinase inhibitors that inhibit activation of an EGFR. Antibodies to EGFR include IgG; IgM; IgA; antibody fragments that retain EGFR binding capability, e.g., Fv, Fab, F(ab)2, single-chain antibodies, and the like; chimeric antibodies; etc. Small molecule tyrosine kinase inhibitors of EGFR include EGFR-selective tyrosine kinase inhibitors. Small molecule tyrosine kinase inhibitors of EGFR can have a molecular weight in a range of from about 50 Da to about 10,000 Da.

Specific exemplary, art-recognized EGFR inhibitors of the instant disclosure include the receptor tyrosine kinase inhibitors erlotinib, gefitinib, afatinib and osimertinib, which have the following structures:

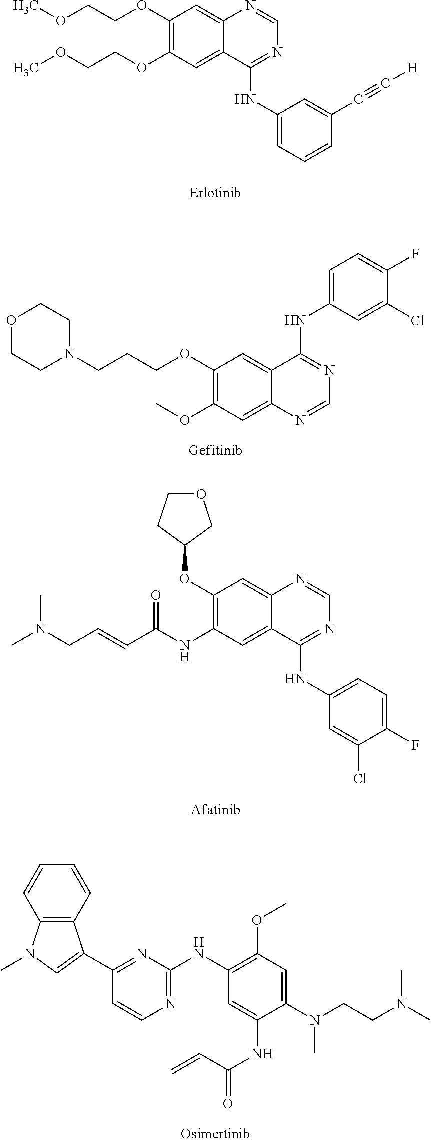

### Combination Therapies

Blocking of immune checkpoints and/or activating co-stimulatory receptors is explicitly contemplated as a means of enhancing (optionally further enhancing) the effects of test agents identified as modulating CD8⁺ T cell killing of target cells, as immune checkpoint blockade and/or activation of co-stimulatory factors can exert broadly overlapping immunomodulatory effects. Indeed, as will be appreciated by one of ordinary skill in the art, agents identified as enhancing CD8⁺ T cell killing of target cells (including, e.g., EGFR inhibitors such as erlotinib, gefitinib, afatinib and osimertinib) can be administered to a subject in combination with other immunomodulatory agents, to achieve combined efficacies. Exemplary agents that are explicitly contemplated for administration in combination with EGFR inhibitory agents (or other agents identified to enhance CD8⁺ T cell killing of target cells) of the current disclosure include anti-PD-1 (PCD1, Programmed Cell Death 1 protein and pathway) agents, anti-CTLA (Cytotoxic T-Lymphocyte Associated Protein proteins and pathways, including CTLA-4) agents, anti-KIR (inhibitory killer IgG-like receptor protein and pathway) agents, anti-TIGIT (T cell immunoreceptor with Ig and ITIM domains protein and pathway) agents, anti-TIM-3 (T cell immunoglobulin and mucin-domain containing-3 or Hepatitis A virus cellular receptor 2 protein and pathway) agents, anti-LAG-3 (Lymphocyte-activation gene 3 protein and pathway) agents, 4-1BB (CD137 or tumor necrosis factor receptor superfamily member 9 protein and pathway) agonists, ICOS (inducible co-stimulator molecule protein or pathway) agonists, GITR (glucocorticoid-induced TNFR-related protein or pathway) agonists and CD28 (Cluster of Differentiation 28 protein or pathway) agonists. Such agents and agonists are most commonly antibody agents; however, small molecules, peptide drugs and other compositions are also contemplated as within the scope of such agents and agonists.

### CRISPR Agents

CRISPR (Clustered regularly interspaced short palindromic repeats)/CRISPR-associated (Cas) systems provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on association of the protein with two naturally occurring or synthetic RNA molecules called crRNA and tracrRNA (also called guide RNAs). In some cases, the two molecules are covalently linked to form a single molecule (also called a single guide RNA (“sgRNA”)). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-strand break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Cas9-like proteins with decreased DNA-cleavage activity (even no DNA-cleaving activity) can still be guided to a target DNA and can block RNA polymerase activity. Thus, enzymatically inactive Cas9-like proteins can be targeted to a specific location in a target DNA by a DNA-targeting RNA in order to block transcription of the target DNA.

Detailed information regarding CRISPR agents can be found, for example in (a) Jinek et. al., Science. 2012 Aug. 17; 337(6096):816-21: “A programmable dual-RNA-guided DNA endonuclease in adaptive bacterial immunity”; (b) Qi et al., Cell. 2013 Feb. 28; 152(5): 1173-83: “Repurposing CRISPR as an RNA-guided platform for sequence-specific control of gene expression”, and (c) U.S. patent application Ser. No. 13/842,859 and PCT application number PCT/US 13/32589; all of which are hereby incorporated by reference in their entirety. Thus, the term “CRISPR agent” as used herein encompasses any agent (or nucleic acid encoding such an agent), comprising naturally occurring and/or synthetic sequences, that can be used in the Cas9-based system (e.g., a Cas9 or Cas9-like protein; any component of a DNA-targeting RNA, e.g., a crRNA-like RNA, a tracrRNA-like RNA, a single guide RNA, etc.; a donor polynucleotide; and the like).

### RNAi Agents, Antisense Agents

As would be recognized by the skilled artisan, RNAi agents (e.g., dsRNAs, shRNAs) and/or antisense agents can also be employed in the screening methods described in the instant disclosure, optionally as an alternative to, or in addition to, CRISPR agents as described herein.

### High-Throughput Immune-Oncology Screen Identifies EGFR Inhibitors as Potent Enhancers of Cytotoxic T Lymphocyte Antigen-Specific Tumor Cell Killing

Described herein is a high-throughput screening assay that is used to identify both drug candidates (plate-based compound screen) and targets (pooled CRISPR/Cas9 screen). Prior studies have paired target cells expressing a model antigen with CD8+ T cells expressing antigen-specific T cell receptors with the intent to identify tumor cell-intrinsic immunomodulatory genes (Manguso et al., 2017 Nature, 547:413-8; Pan et al., 2018 Science, eaao1710; Patel et al., 2017 Nature, 548:537-42). Insofar as these studies elucidated mechanisms conferring resistance to immune pressure, the results presented herein are largely concordant, whether from the compound screen (JAK2 inhibitor AZD1480) or CRISPR/Cas9 screen (H2-K1, Tap1, Tap2, and B2m). Yet, where these other studies focused on the fundamental biology and specific pathways that tumor cells often mutate or downregulate to evade T cell recognition and killing, results presented herein focus on the opposite end: genes that sensitize tumor cells to CD8+ T cell-mediated killing.

EGFR was an unexpected hit. EGFR has previously been shown to antagonize HLA class-I expression via suppression of STAT1 in head and neck cancer patients treated with cetuximab (Srivastava et al., 2015 Cancer Immunol Res, 3:936-45). Cetuximab-mediated inhibition of EGFR signaling was associated with enhanced IFN-γ receptor 1 (IFNAR) expression which, through STAT1-dependent signaling, enhanced IFN-γ-induced expression of HLA class-I and TAP1/2. In another study, pharmacological inhibitors of EGFR and cetuximab were shown to upregulate basal and IFN-γ-induced expression of class I and class II in human keratinocytes. The same study provided in vivo validation whereby patients already receiving erlotinib or cetuximab consented to skin biopsies, demonstrating modest on-treatment elevation in HLA mRNA (Pollack et al., 2011 Clin Cancer Res, 17:4400-13). A recent genome-wide CRISPR screen characterizing mechanisms of tumor cell immune evasion identified SOX10 as a top hit conferring resistance to T cell-mediated killing commensurate with B2m, HLA-A, and TAP1 (Patel et al., 2017 Nature, 548:537-42). This would plausibly implicate an EGFR-related mechanism, as knockdown of SOX10 in human melanoma was previously shown (Sun et al., 2014 Nature, 508:118-22) to result in high expression of EGFR, which would dampen antigen processing and presentation, leading to immune escape.

Any modulation of antigen presentation or tumor cell “stress” is likely to affect NK cell involvement in the anti-tumor immune response. Pharmacologic inhibition of EGFR with gefitinib or silencing with siRNA increased expression of MHC-I in the PC9 mutEGFR T790M human NSCLC cell line, which is consistent with the data presented herein, and downregulated expression of NKG2D ligands MICB and ULBP-2/5/6 (He et al., 2013 J Transl Med, 11:186). Subsequently, gefitinib attenuated NK cell-mediated lysis of tumor cells. In another study, however, EGFR inhibition with gefitinib enhanced NK cell-mediated cytotoxicity of L858R+T790M mutEGFR tumor cells via upregulation of NKG2D ligands MICA, ULBP1, and ULBP2 (Morvan M G and Lanier L L. 2016 Nat Rev Cancer, 16:7-19). EGFR inhibition could potentially enhance or inhibit NK cell recognition of tumor cells by modulation of stress ligands recognized by activating NK cell receptors and through KIR-mediated “missing self” recognition that is dependent upon expression of MHC class I (Mok et al., 2009 N Engl J Med, 361:947-57). It is possible that there are alternative mechanisms of EGFR inhibitor-mediated immunomodulatory function that involve NK cells.

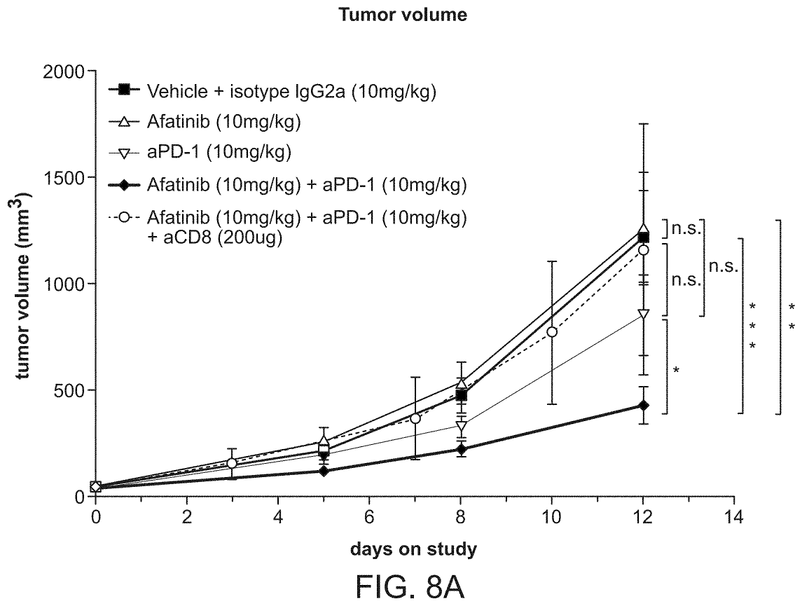

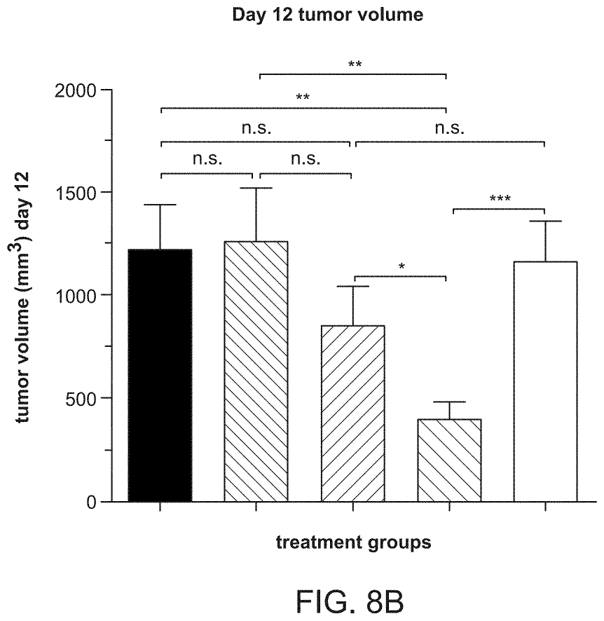

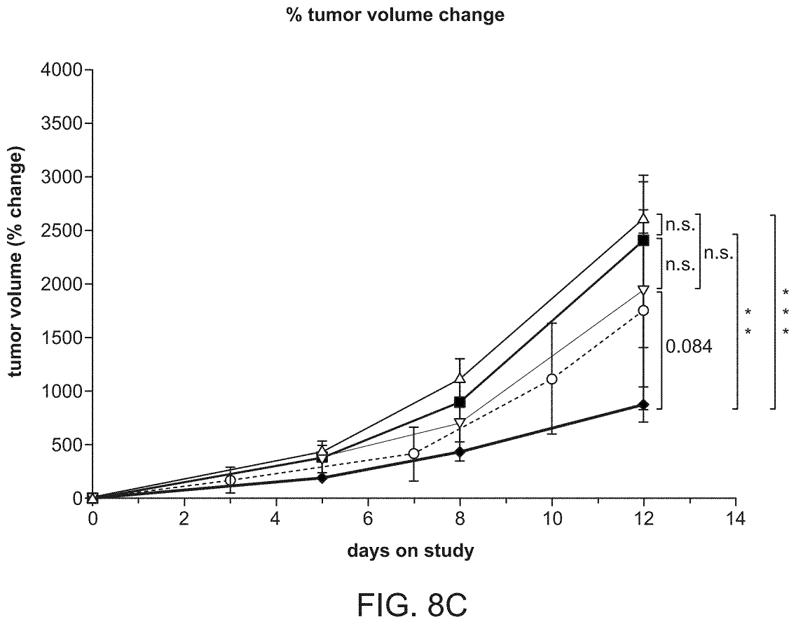

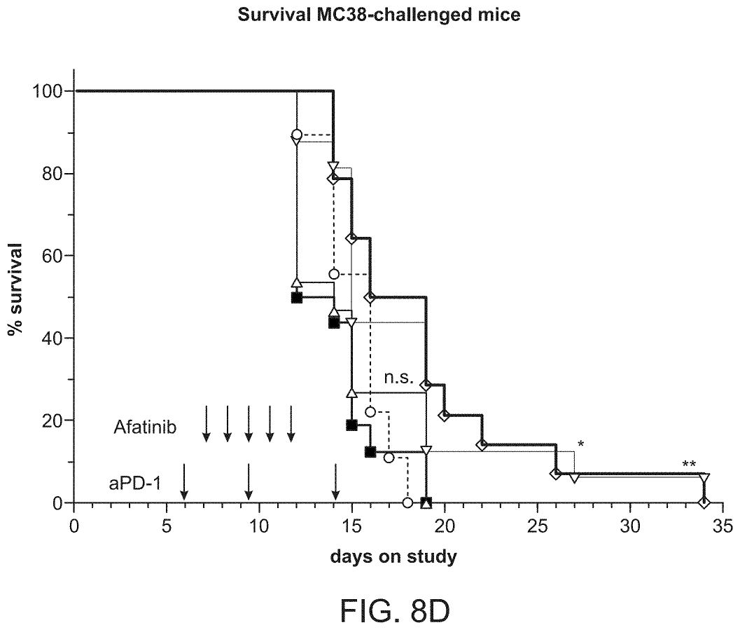

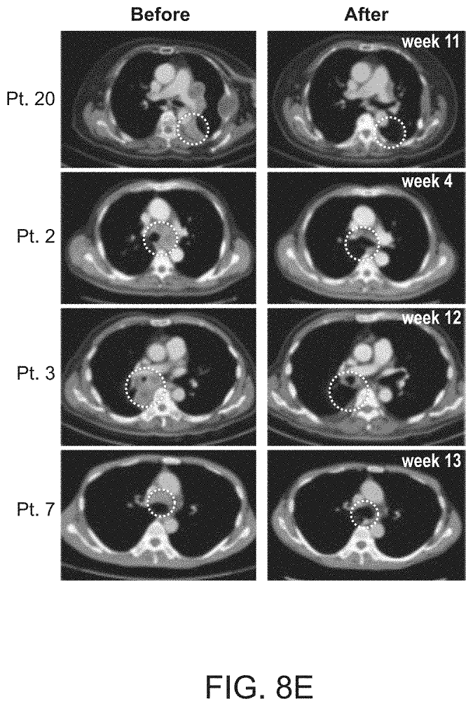

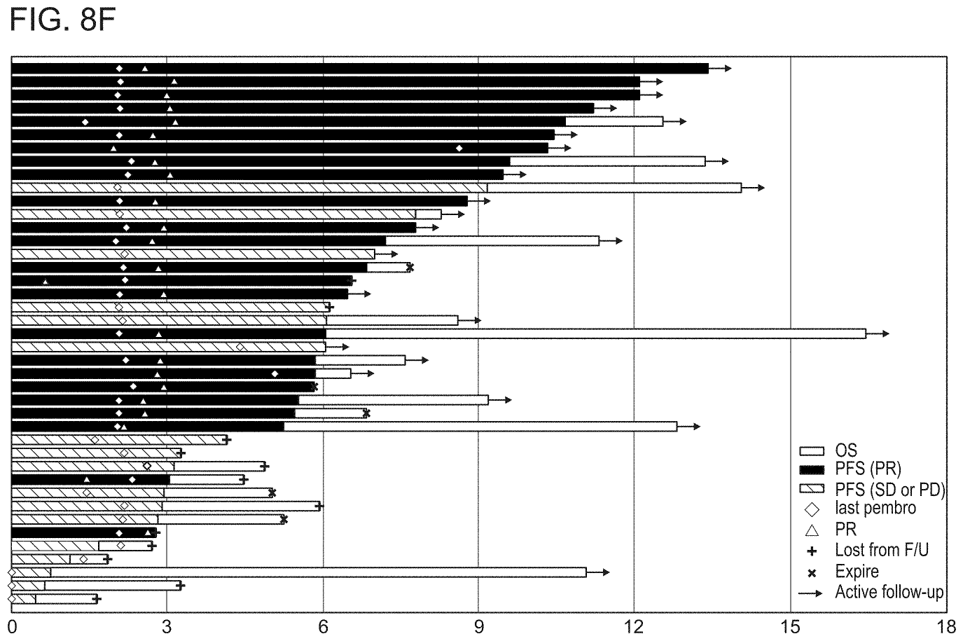

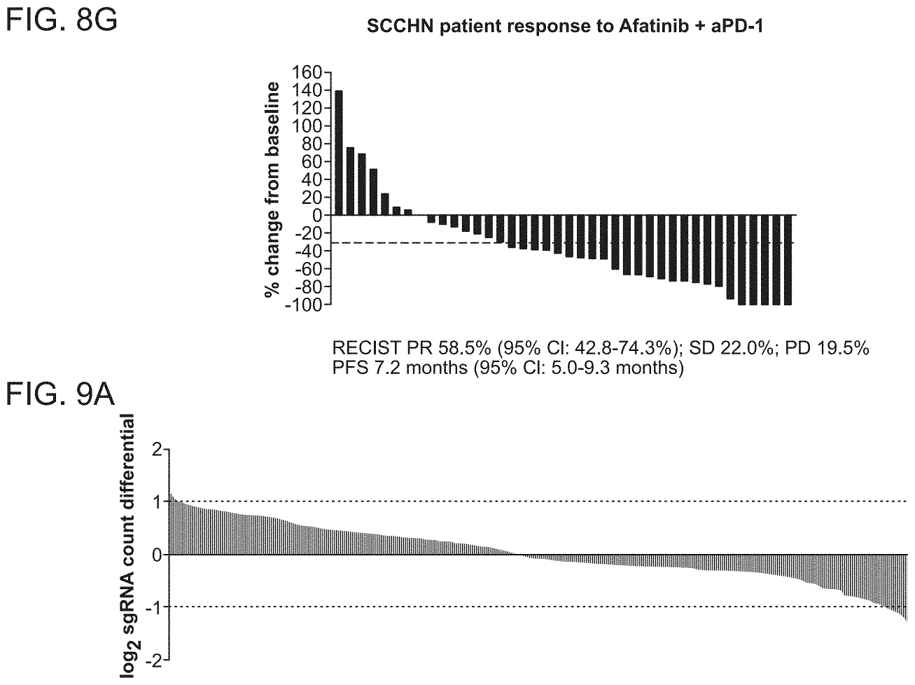

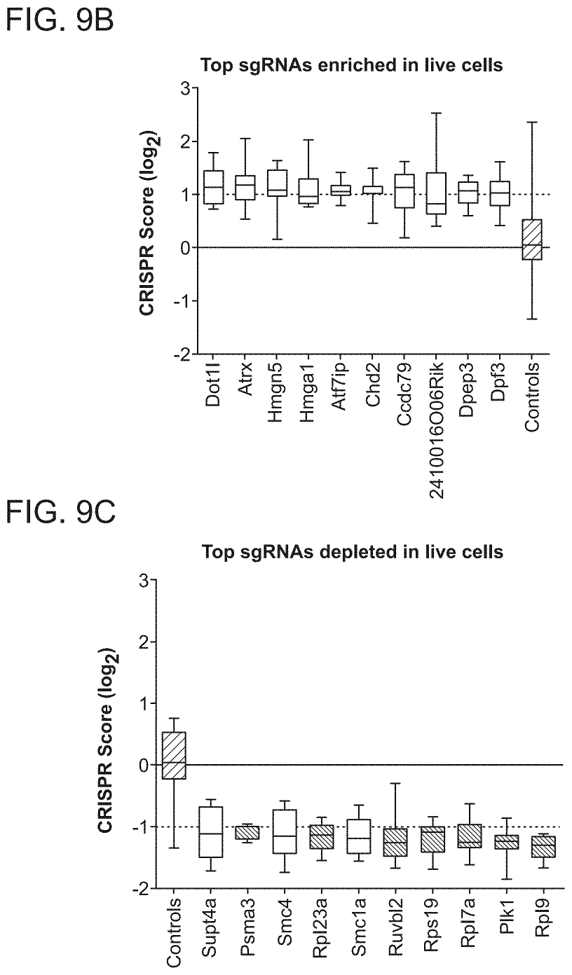

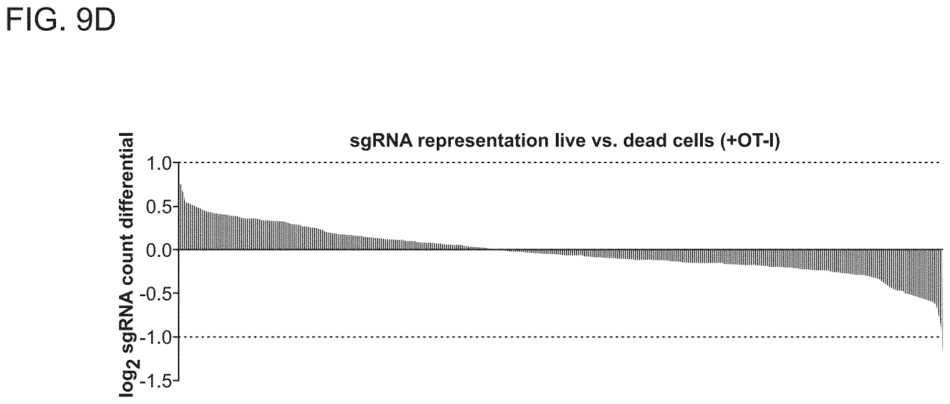

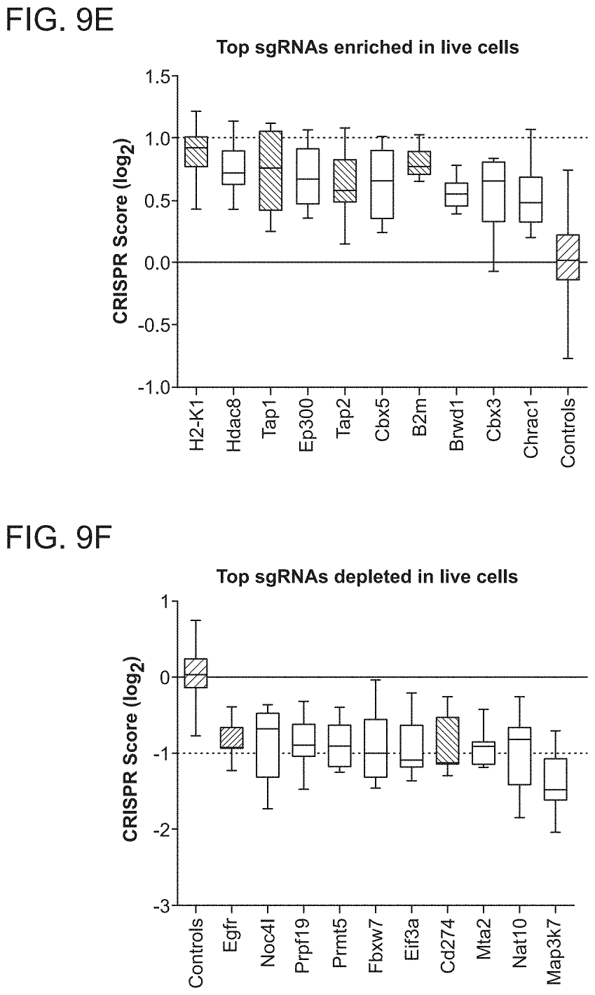

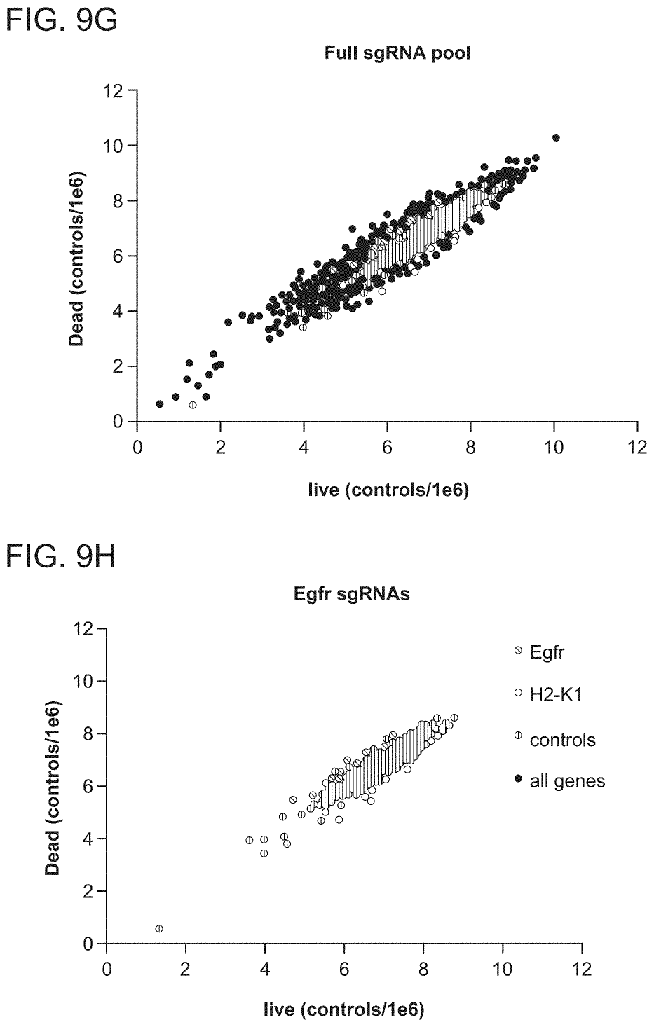

EGFR TKIs exhibit minimal therapeutic efficacy against wtEGFR NSCLC (Shepherd et al., 2005 N Engl J Med, 353:123-32; Townsley et al., 2006 Br J Cancer, 94:1136-43), colorectal cancer (Chen et al., 2015 J Thorac Oncol Off Publ Int Assoc Study Lung Cancer, 10:910-23), and SCCHN (Manguso et al., 2017 Nature, 547:413-8). This clinical evidence, combined with the synergistic effect of afatinib and anti-PD-1 in the in vivo model described herein suggests that combination of immune checkpoint blockade with EGFR TKI may have limited therapeutic benefit in wtEGFR tumors. Yet, synergistic efficacy was observed in a cohort of SCCHN patients, with clear post-treatment reductions in tumor burden (FIG. 9E), ongoing responses (FIG. 8F), and ORR of 58.5% (FIG. 8G).

The immunological contribution of oncogenic EGFR has been explored clinically (Chen et al., 2015 J Thorac Oncol Off Publ Int Assoc Study Lung Cancer, 10:910-23) and pre-clinically (Akbay et al., 2013 Cancer Discov, 3:1355-63), but mostly as it relates to its regulation of PD-L1 expression in tumor cells. This led to the hypothesis that addition of PD-1/PD-L1 blocking antibodies might improve EGFR tyrosine kinase inhibitor (TKI) in EGFR-mutant lung cancer by activating the immune infiltrate otherwise suppressed by secondary mechanisms downstream of aberrant EGFR signaling. There are two clinical trials exploring combination PD-1 blockade with an EGFR inhibitor in EGFR mutant lung cancer: nivolumab plus EGF816 (NCT02323126) and nivolumab plus erlotinib (CheckMate 012 NCT01454102). A trial of osimertinib, a mutant selective EGFR inhibitor, combined with the PD-L1 inhibitor durvalumab, in patients with EGFR mutant lung cancer was stopped due to toxicity (NCT02454933). Yet, EGFR mutant lung cancer, the largest cohort of patients treated with EGFR inhibitors, may not be an ideal setting in which positive immunomodulatory properties would necessarily be noticed, largely due to the immunologically “cold” nature of the disease, as shown previously (Lizotte et al., 2016 JCI Insight, 1(14): e89014. Prior to the invention described herein, all the EGFR/checkpoint blockade combinations have been focused on EGFR mutant lung cancer. In fact, only afatinib has an approval in a non-EGFR mutant setting. The data presented above confirming EGFR as an immune-oncology target was conducted in three distinct EGFR WT models. This suggests that, whether through its regulation of PD-L1 or suppression of basal and IFN-γ-induced antigen processing and presentation, inhibition of EGFR may be broadly efficacious across mutEGFR and wtEGFcancers. The oncogenic properties of EGFR are well-established, but the results presented herein supports the classification of EGFR as an immune-oncology target. Given the FDA-approval of multiple pharmacologic and biologic inhibitors of EGFR and their established clinical application, inclusion of inhibitors to non-mutated EGFR is an attractive approach to amplify the immunogenicity of tumors treated with immune checkpoint blockade. The human data in SCCHN is evidence for the utility of this approach (FIG. 8E and FIG. 8F)

Yet EGFR TKI trials report high incidences of adverse events such as skin rashes in 66-90% of patients (Shepherd et al., 2005 N Engl J Med, 353:123-32; Townsley et al., 2006 Br J Cancer, 94:1136-43; Chen et al., 2015 J Thorac Oncol Off Publ Int Assoc Study Lung Cancer, 10:910-23; DuPage et al., 2011 Cancer Cell, 19:72-85). There is ample evidence to support the assertion that these drugs induce upregulation of MHC class I. This could potentially cause aberrant T cell recognition of self-antigen. Breaking of tolerance may explain the high rates of toxicity observed with EGFR TKI; they may be immune-mediated. Immune activation may also explain the therapeutic benefit observed in wtEGFR lung and colorectal patients treated with EGFR TKI (Shepherd et al., 2005 N Engl J Med, 353:123-32; Townsley et al., 2006 Br J Cancer, 94:1136-43). Adverse events are likely to remain consistent, if not become exacerbated, by combination with immune checkpoint blockade. It is noted in the limited dataset of 41 patients that this compounded toxicity was not observed with combination therapy. Recommended dosages of EGFR TKIs are intended to inhibit constitutively high expression of EGFR resulting from activating mutations. Synergistic efficacy could be maintained and potential combination toxicity mitigated by using EGFR inhibitors at lower dosages, particularly in wtEGFR patients, or by more intelligent sequencing.

Described herein is an assay for high throughput screening that can be utilized to identify new immunomodulatory therapeutics and current drugs that would logically be expected to augment immune checkpoint blockade and other developing immunotherapies. As currently designed, one OT-I mouse spleen with a routine harvest of 10-12 million CD8+ T cells is sufficient to plate 10-12 96-well assay plates, rendering analysis of compound libraries in the hundreds to thousands highly feasible. The initial screen identified EGFR as a target that sensitizes tumor cells to CD8+ T cell-mediated killing, a result which was confirmed in two different murine tumor cell lines and independently validated in a pooled CRISPR-Cas9 screen. Additionally, inhibition of EGFR with afatinib enhanced anti-PD-1 therapeutic efficacy in vivo in the MC38 syngeneic colon cancer model and in human SCCHN patients. The CTL OT-I assay is a tool to rationally identify promising drug combinations to enhance immunotherapy, which is rapidly becoming a cornerstone of medical oncology.

### Pharmaceutical Compositions, Kits, and Administration

The present disclosure provides pharmaceutical compositions comprising an agent described herein (e.g., an EGFR inhibitor, or a pharmaceutically acceptable salt, solvate, hydrate, polymorph, co-crystal, tautomer, stereoisomer, isotopically labeled derivative, or prodrug thereof), and optionally a pharmaceutically acceptable excipient. In certain embodiments, the pharmaceutical composition described herein comprises an immunomodulatory agent (e.g., an EGFR inhibitor), or a pharmaceutically acceptable salt thereof, and optionally a pharmaceutically acceptable excipient. In certain embodiments, the pharmaceutical composition described herein comprises an immunomodulatory agent (e.g., an EGFR inhibitor), or a pharmaceutically acceptable salt thereof, and a pharmaceutically acceptable excipient.

In certain embodiments, the immunomodulatory agent described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating and/or preventing a disease (e.g., a disease described herein) in a subject in need thereof. In certain embodiments, the effective amount is an amount effective for treating a disease in a subject in need thereof. In certain embodiments, the effective amount is an amount effective for preventing a disease in a subject in need thereof. In certain embodiments, the effective amount is an amount effective for reducing the risk of developing a disease in a subject in need thereof. In certain embodiments, the effective amount is an amount effective for male contraception (e.g., effective for inhibiting sperm formation) in a subject in need thereof. In certain embodiments, the effective amount is an amount effective for inhibiting the replication of a virus. In certain embodiments, the effective amount is an amount effective for killing a virus. In certain embodiments, the effective amount is an amount effective for enhancing the activity (e.g., augmenting CTL killing activity upon target cells) of CTLs in a subject or cell. In certain embodiments, the effective amount is an amount effective for inhibiting the activity (e.g., reducing CTL killing activity upon target cells) of CTLs in a subject or cell in a subject or cell. In certain embodiments, the effective amount is an amount effective for selectively enhancing the killing of target cells by effector cells (e.g., CTLs) by at least two-fold in a subject or cell culture, as compared to an appropriate control.

An effective amount of an agent may vary from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

In certain embodiments, the effective amount is an amount effective to selectively enhance CTL-mediated killing of target cells displaying a targeted MHC-I antigen by at least about 10%, at least about 20%, at least about 30%, at least about 40%, at least about 50%, at least about 60%, at least about 70%, at least about 80%, at least about 90%, at least about 100%, at least about 200%, at least about 300%, at least about 500%, or at least about 1000%. In certain embodiments, the effective amount is an amount effective for inhibiting CTL-mediated killing of target cells displaying a targeted MHC-I antigen by at least about by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfate, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., an EGFR inhibitor) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter of less than 7 nanometers. Alternatively, at least 95% of the particles by weight have a diameter greater than 1 nanometer and at least 90% of the particles by number have a diameter of less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Immunomodulatory agents provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., an EGFR inhibitor) described herein.

As noted elsewhere herein, An immunomodulatory agent of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The immunomodulatory agent is administered to the subject in an amount sufficient to achieve a desired effect at a desired site (e.g., enhanced CTL-mediated killing of target cells) determined by a skilled clinician to be effective. In some embodiments of the disclosure, the immunomodulatory agent is administered at least once a year. In other embodiments of the invention, the immunomodulatory agent is administered at least once a day. In other embodiments of the invention, the immunomodulatory agent is administered at least once a week. In some embodiments of the invention, the immunomodulatory agent is administered at least once a month.

Exemplary doses for administration of an immunomodulatory agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and an imaging and/or therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., an EGFR inhibitor) described herein. In certain embodiments, a dose described herein includes independently between 1 mg and 3 mg, inclusive, of an agent (e.g., an EGFR inhibitor) described herein. In certain embodiments, a dose described herein includes independently between 3 mg and 10 mg, inclusive, of an agent (e.g., an EGFR inhibitor) described herein. In certain embodiments, a dose described herein includes independently between 10 mg and 30 mg, inclusive, of an agent (e.g., an EGFR inhibitor) described herein. In certain embodiments, a dose described herein includes independently between 30 mg and 100 mg, inclusive, of an agent (e.g., an EGFR inhibitor) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., an EGFR inhibitor) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. a subject or cell. In certain embodiments, a pharmaceutical composition described herein including an agent (e.g., an EGFR inhibitor) described herein and an additional pharmaceutical agent shows a synergistic effect that is absent in a pharmaceutical composition including one of the agent and the additional pharmaceutical agent, but not both.

In some embodiments of the invention, a therapeutic agent distinct from the immunomodulatory agent is administered prior to, in combination with, at the same time, or after administration of the imaging and/or therapeutically effective amount of an immunomodulatory agent of the disclosure. In some embodiments, the second therapeutic agent is selected from the group consisting of a chemotherapeutic, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, other immunomodulatory agents, anti-cancer agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or agent described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or agent described herein. In some embodiments, the pharmaceutical composition or agent described herein provided in the first container and the second container are combined to form one unit dosage form.

Thus, in one aspect, provided are kits including a first container comprising an agent (e.g., an EGFR inhibitor) described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the kits are useful for treating and/or preventing a disease described herein in a subject in need thereof. In certain embodiments, the kits are useful for treating a disease described herein in a subject in need thereof. In certain embodiments, the kits are useful for preventing a disease described herein in a subject in need thereof. In certain embodiments, the kits are useful for reducing the risk of developing a disease described herein in a subject in need thereof. In certain embodiments, the kits are useful for male contraception. In certain embodiments, the kits are useful for inhibiting sperm formation. In certain embodiments, the kits are useful for in inhibiting the replication of a virus. In certain embodiments, the kits are useful for killing a virus. In certain embodiments, the kits are useful for enhancing the activity (e.g., CTL-mediated target cell killing) in a subject or cell. In certain embodiments, the kits are useful for inhibiting the activity (e.g., CTL-mediated target cell killing) of CTL cells in a subject or cell.

In certain embodiments, the kits are useful for screening a library of agents to identify an agent that is useful in a method of the disclosure.

In certain embodiments, a kit described herein further includes instructions for using the kit, such as instructions for using the kit in a method of the disclosure (e.g., instructions for administering an agent (e.g., an EGFR inhibitor) or pharmaceutical composition described herein to a subject). A kit described herein may also include information as required by a regulatory agency such as the U.S. Food and Drug Administration (FDA). In certain embodiments, the information included in the kits is prescribing information. In certain embodiments, the kits and instructions provide for treating and/or preventing a disease described herein in a subject in need thereof. In certain embodiments, the kits and instructions provide for treating a disease described herein in a subject in need thereof. In certain embodiments, the kits and instructions provide for preventing a disease described herein in a subject in need thereof. In certain embodiments, the kits and instructions provide for reducing the risk of developing a disease described herein in a subject in need thereof. In certain embodiments, the kits and instructions provide for male contraception. In certain embodiments, the kits and instructions provide for inhibiting the replication of a virus. In certain embodiments, the kits and instructions provide for killing a virus. In certain embodiments, the kits and instructions provide for inducing apoptosis of an in vitro cell. In certain embodiments, the kits and instructions provide for inducing apoptosis of a cell in a subject. In certain embodiments, the kits and instructions provide for inducing G1 arrest in a subject or cell. In certain embodiments, the kits and instructions provide for screening a library of agents to identify an agent (e.g., an EGFR inhibitor) that is useful in a method of the disclosure. A kit described herein may include one or more additional pharmaceutical agents described herein as a separate composition.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Generation of Luciferized ID8 Cell Lines

A firefly luciferase-OVA fusion cassette was cloned from the Lenti-LucOS vector previously described (DuPage et al. Cancer Cell 19: 72-85) using two-step PCR (Fu et al. Nucleic Acids Res. 36: e54) with primers as follows: attL1 forward 5′-AGGCTCCTGCAGGACCATGGAAGACGCCAAAAAC-3′ (SEQ ID NO: 1); attL2 reverse 5′-GAAAGCTGGGTCTCGAGCTAGCGGCCGCTTACAAG-3′ (SEQ ID NO: 2); attL1-T1 forward 5′-CCCCGATGAGCAATGCTTTTTTATAATGCCAACTTTGTACAAAAAAGCAGGCTCCTGC AGGACCATG-3′ (SEQ ID NO: 3); attL2-T1 reverse 5′-GGGGGATAAGCAATGCTTTCTTATAATGCCAACTTTGTACAAGAAAGCTGGGTCTCGA GCTA-3′ (SEQ ID NO: 4). PCR product containing lucOS ORF was then inserted into pLVX-IRES-Neo lentiviral vector (Clontech, Mountain View, Calif.) using Gateway® LR Clonase® II (Thermo Fisher, Waltham, Mass.). A Renilla luciferase vector was constructed using the same protocol and also inserted into the pLVX-IRES-Neo lentiviral vector. Plasmids were transformed into One Shot® OmniMAX™ 2 competent cells according to the manufacturer's protocol (Thermo Fisher, Waltham, Mass.). Clones were miniprepped (Qiagen, Valencia, Calif.), genotyped by PCR, sequence-verified, and transiently transfected into 293T cells to assess firefly luciferase expression. Positive clones were co-transfected into 293T cells along with d8.9 and VSV-G packaging plasmids. ID8-Cas9 cells were transduced with pLVX-lucOS-IRES-Neo or pLVX-rluc-IRES-Neo vectors and placed under G418 selection for seven days. Viral production and ID8 spin-fection were conducted according to the Broad Institute's lentiviral production guidelines (Yang et al. Nat. Methods 8: 659-661). Clonal cell lines of “lucOS” and “rluc” cell lines were generated by limiting dilution, expanded, and verified for luciferase and OVA expression.

### Harvesting and Activation of OT-I T Cells

C57BL/6-Tg(TcraTcrb)1100Mjb/J stock #003831 “OT-I” mice (Jackson labs, Bar Harbor, Me.) were bred in-house. 8-12 week old mice were sacrificed and spleens were harvested by mechanical separation through a 40 μM filter. Red blood cells were lysed using 1×RBC lysis buffer (Biolegend, San Diego, Calif.). Splenic single cell suspension was resuspended in TruStain fcX™ (anti-mouse CD16/32) FcR blockade diluted 1:100 in FACS buffer (PBS+2% FBS) and incubated on ice for 15 min. CD8⁺ T cells were stained with mouse CD8 (Ly-2) MicroBeads for 20 min, washed with FACS buffer, and isolated using magnetic separation and LS columns according to manufacturer's protocol (Miltenyi Biotec, San Diego, Calif.). CD8⁺ T cells were eluted into RPMI (Life Technologies, Carlsbad, Calif.)+10% FBS (HyClone, Logan, Utah) and pen/strep (Life Technologies, Carlsbad, Calif.). OT-I CD8⁺ T cells were then activated with Dynabeads Mouse T-Activator CD3/CD28 beads (Life Technologies, Carlsbad, Calif.) for 24 hr before addition to lucOS/rluc co-cultures.

### OT-I CTL Assay

10,000 ID8-lucOS and 10,000 ID8-rluc cells were plated in 1004, of cell culture media (DMEM+10% FBS+pen/strep) in solid white, flat-bottomed, tissue culture-treated 96-well plates (Thermo Fisher, Waltham, Mass.). Overnight-stimulated OT-I CD8⁺ T cells were then added at the designated Effector:Target ratios with or without compounds in a total volume of 200 μL. Plates were incubated for 48 hr at 37° C. and 5% CO₂. After 48 hr timepoint, 1004, of media was removed prior to dual-luciferase assay. Briefly: 504, of homemade Dual-Glo® luciferase buffer (Lu et al. Science 343: 305-309) was added to wells and incubated for 30 min before analysis of Firefly luminescence; then 504, of Dual-Glo® Stop & Glo® buffer was added, plates were incubated for 30 min, then analyzed for Renilla luminescence. An EnSpire plate reader (PerkinElmer, Waltham, Mass.) was used for quantification of luminescence. 203 compounds (listed above; details for LINCS kinase library can be found at lincs.hms.harvard.edu/db/sm/) were screened in high-throughput at 1 μM final concentration in duplicate, in 96-well plates in a first set that contained both OT-I CTLs and ID8 target cells and in a second set that contained ID8 target cells only. No compounds were placed in edge wells and all plates contained multiple DMSO control cells. Hits were calculated based on ΔΔCt method whereby ΔΔCt=(FLuc_{DMSO}/RLuc_{DMSO})/(FLuc_{Compound}/RLuc_{Compound}) where values >1 were assessed as having augmented CTL killing, values ˜1 were assessed as having negligible immunomodulatory effect, and values <1 were assessed as having inhibited CTL killing. Hits from the high-throughput screen were validated with dose response curves using the indicated drug concentrations.

### OT-I IFN-γ ELISA

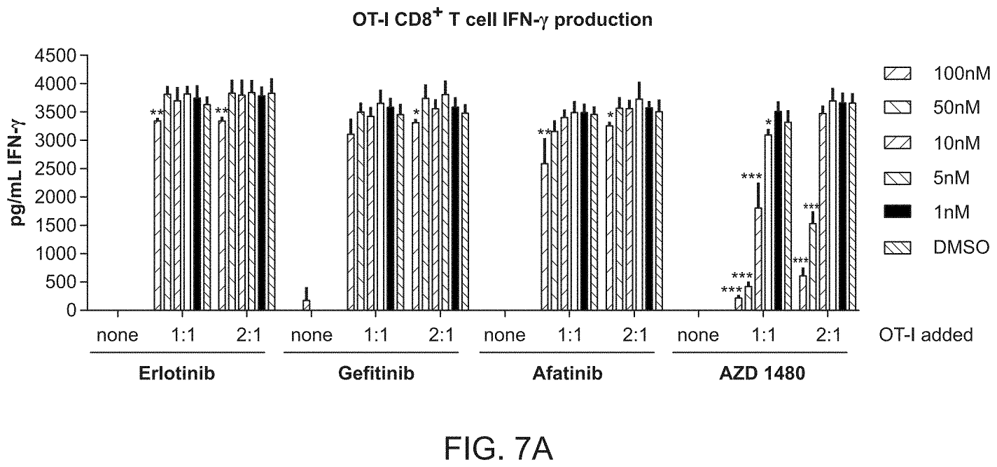

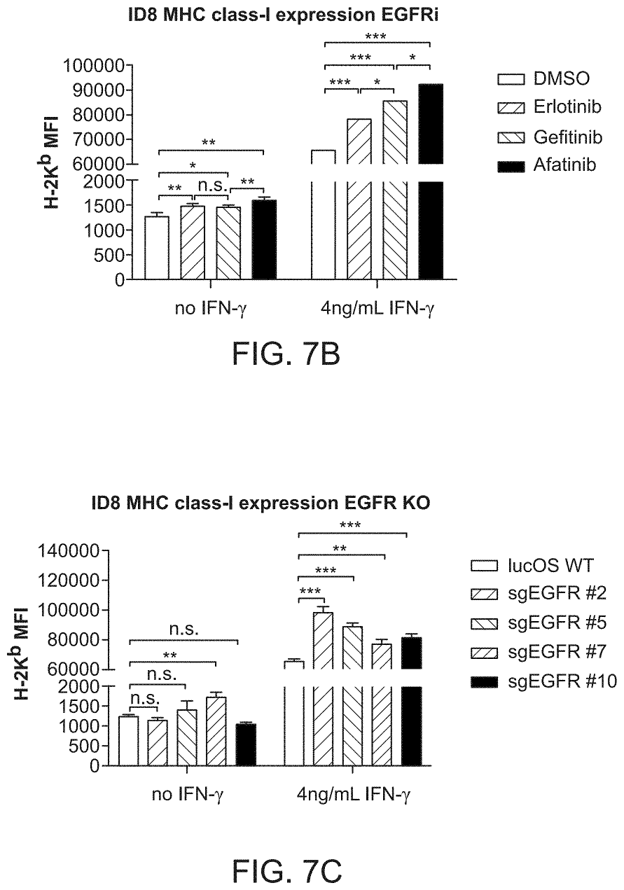

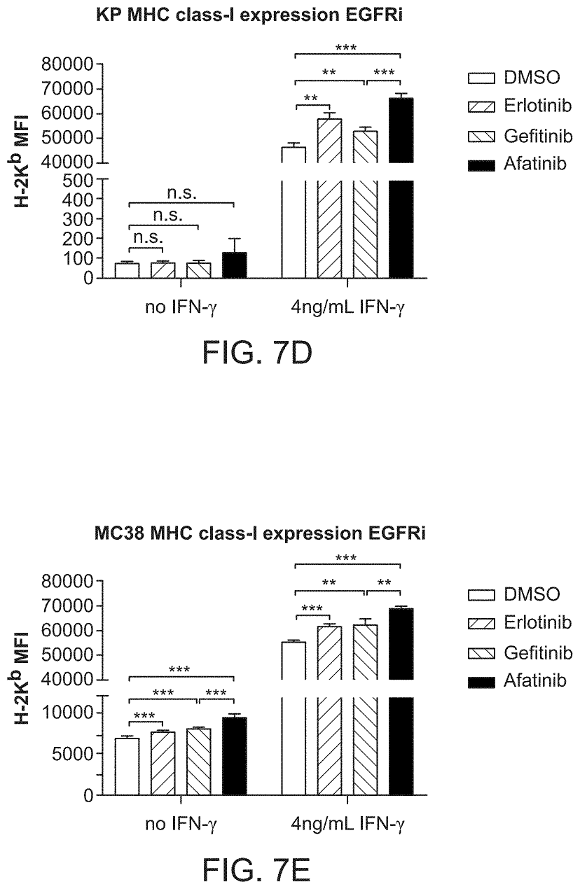

The supernatants from OT-I CTL assays, as described above, were harvested at the 48 hr timepoint prior to dual luciferase assay and analyzed for IFN-γ secretion by LEGEND MAX™ Mouse IFN-γ ELISA Kit (Biolegend, San Diego, Calif.) according to the manufacturer's protocol. Indicated compounds of FIG. 7A-FIG. 7E (erlotinib, gefitinib, afatinib and AZD 1480) were tested at 100, 50, 10, 5, and 1 nM, alongside DMSO only control wells, with compound concentration ranges tested either with no OT-I CTLs or at Effector:Target ratios of 1:1 and 2:1. Conditions were assayed with four replicate wells per experiment.

### Flow Cytometry of MHC Class-I Expression

100,000 ID8-lucOS, ID8-lucOS sgEGFR KO, Kras^{G12D};p53^{-/-}, and MC38 cell lines were cultured in 12-well plates in 2 mL of culture media (DMEM+10% FBS+pen/strep) alone or supplemented with 4 ng/mL recombinant mouse IFN-γ (Biolegend, San Diego, Calif.) for 48 hr. Where indicated, cells were treated with erlotinib, gefitinib, or afatinib at a concentration of 100 nM. EGFR KO had loss of EGFR confirmed by Western blot prior to assay. Cells were trypsinized, washed, and resuspended in FACS buffer (PBS+2% FBS) with H-2K^{b}-APC (clone AF6-88.5, Biolegend) at a dilution of 1:100 for 15 min on ice, then washed twice prior to analysis on a BD LSRFortessa with FACSDiva software (BD Biosciences, San Jose, Calif.). Data were analyzed using FlowJo (Ashland, Oreg.) software version 10.0.9.

### CRISPR/Cas9 Screen

ID8-lucOS cells stably expressing Cas9 were transduced with a ˜8000 guide pooled sgRNA library with 10 sgRNA/gene covering: 87 control genes (essential genes, oncogenes, tumor suppressor genes), 86 immune modulators (immune checkpoints, differentially regulated immune genes), 524 epigenetic regulators, 34 MHC genes, and 500 non-targeting sgRNA. sgRNAs were expressed from the pXPR-sgRNA-2A-GFP vector (Addgene, Cambridge, Mass.) at MOI of 0.3 and selected for blasticidin resistance at a representation of 500 cells/sgRNA, which was maintained throughout the screen. OT-I T cells were harvested and pre-stimulated as in plated-based compound screen and added to T175 flasks with monolayers of sgRNA-transduced ID8-lucOS cells at an E:T ratio of 1:1 or without OT-I T cells. Cell cultures were maintained for 72 hr, at which point live and dead ID8-lucOS cells were harvested for isolation of genomic DNA. Genomic DNA from cell pellets was extracted using DNeasy Blood and Tissue Kit (Qiagen, Carlsbad, Calif.) and concentrated using Genomic DNA Clean & Concentrator (Zymo Research, Irvine, Calif.), both according to manufacturers' protocol. Twelve μg gDNA (250× representation for 8000 sgRNAs at 6 pg DNA/cell) was amplified using Titanium Taq DNA Polymerase (Clontech, Mountain View, Calif.) in one-step PCR reaction with following parameters: 95° C. 1 min, [95° C. 30 sec, 64° C. 30 sec, 72° C. 30 sec]×22 cycles, 72° C. 5 min first step with F2/R2 primers. PCR products were verified on DNA1000 Bioanalyzer (Agilent, Santa Clara, Calif.) and ˜350 bp bands gel purified using QIAquick Gel Extraction Kit (Qiagen, Carlsbad, Calif.). PCR products were diluted to 10 ng/μL, pooled, and sequenced on NextSeq machine (Illumina, San Diego, Calif.).

### In Vivo Validation

C57BL/6J stock #000664 mice (Jackson labs, Bar Harbor, Me.) were challenged subcutaneously with 500,000 MC38 colon cancer cells on their flanks and “enrolled” on-study when tumors reached 50 mm³. Mice were treated with vehicle+10 mg/kg IgG2a isotype control (Bio X Cell, West Lebanon, N.H.), 10 mg/kg aPD-1 (clone RMP1-14, Bio X Cell, West Lebanon, N.H.), 10 mg/kg afatinib (Selleck, Houston, Tex.), combination 10 mg/kg aPD-1 and 10 mg/kg afatinib, or combination 10 mg/kg aPD-1 and 10 mg/kg afatinib and 200 μg aCD8α (clone 53-6.7, Bio X Cell, West Lebanon, N.H.). Animals received IP injections of aPD-1 on days 5, 8, and 12 and afatinib on days 6, 7, 8, 9, and 10 (as indicated). Depleting aCD8α was administered two days prior to first aPD-1 treatment. Mice used in experiments were 7-8 weeks of age at time of tumor challenge. Endpoint was considered to be when tumors reached a size of 2000 mm³ or as mandated by institutional guidelines due to development of necrotic lesions.

### Data Analysis

The following denote statistical significance: *p-value <0.05; **p-value <0.01; *** p-value <0.001. Flank tumor growth curves were analyzed using two-way ANOVA, all bar graphs were analyzed using unpaired Student's t-test, and survival experiments used the log-rank Mantel-Cox test for survival analysis. Statistics were calculated using PRISM 7.01 (Graphpad, La Jolla, Calif.).

### Afatinib and Pembrolizumab Combination Therapy

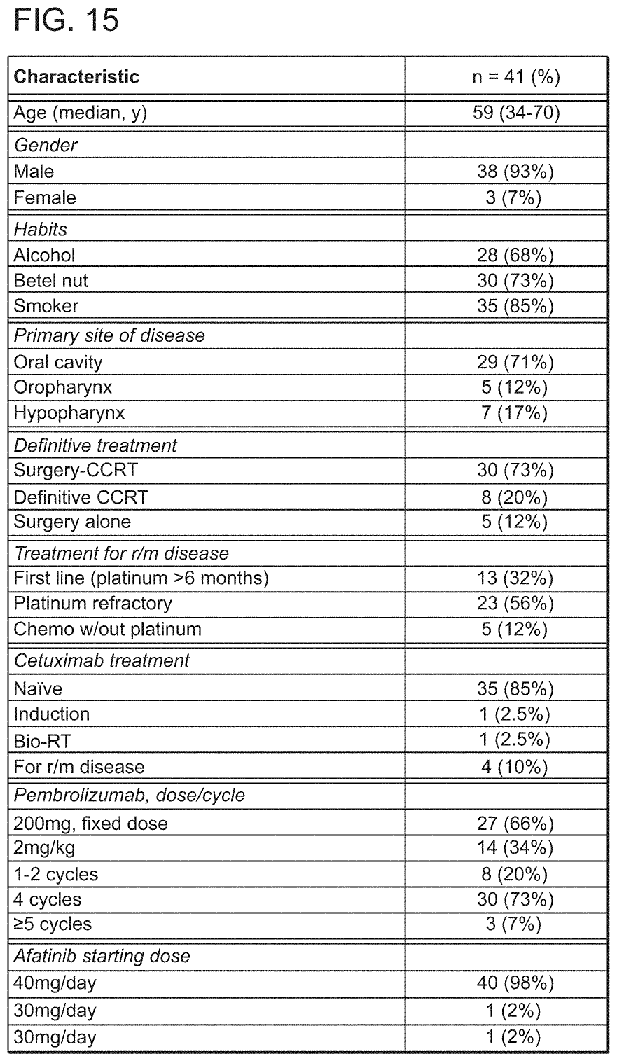

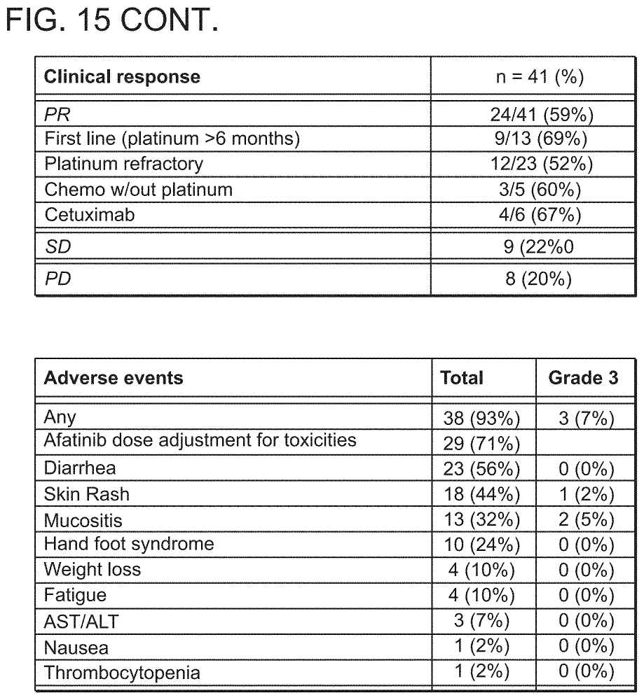

Retrospective medical record and image review was performed of patients with recurrent and/or metastatic squamous cell carcinoma of the oral cavity, oropharynx, hypopharynx, or larynx (r/m SCCHN) who received combination afatinib and pembrolizumab at National Taiwan University Hospital between Nov. 1, 2016 and Sep. 30, 2017 with follow-up through Mar. 30, 2018. Exclusion criteria included prior treatment with afatinib, pembrolizumab, or nivolumab as a monotherapy, or prior treatment with other anti-cancer agents in combination with afatinib or pembrolizumab. Disease status was assessed by MRI or CT scan. In all, 41 patients were eligible for analysis, with clinical annotation and treatment regimen available in (FIG. 15).

### Cell Lines

ID8 were obtained from the laboratory of Gordon Freeman (DFCI) in 2014, MC38 were purchased from ATCC in 2015, 293T were purchased from Invitrogen in 2011, and the Kras^{G12D};p53^{-/-} cell line was derived in-house from the mouse model (Pollack et al., 2011 Clin Cancer Res, 17:4400-13) in 2016. All cell lines were confirmed to be mycoplasma negative by Charles River Research Animal Diagnostic Services using standard Quantitative Fluorescence PCR (QF-PCR) protocol. Only cell lines of <20 passages were used for experiments.

### Example 2: High-Throughput Compound Screen for Modulators of OT-I CTL-Mediated Killing of Target Cells

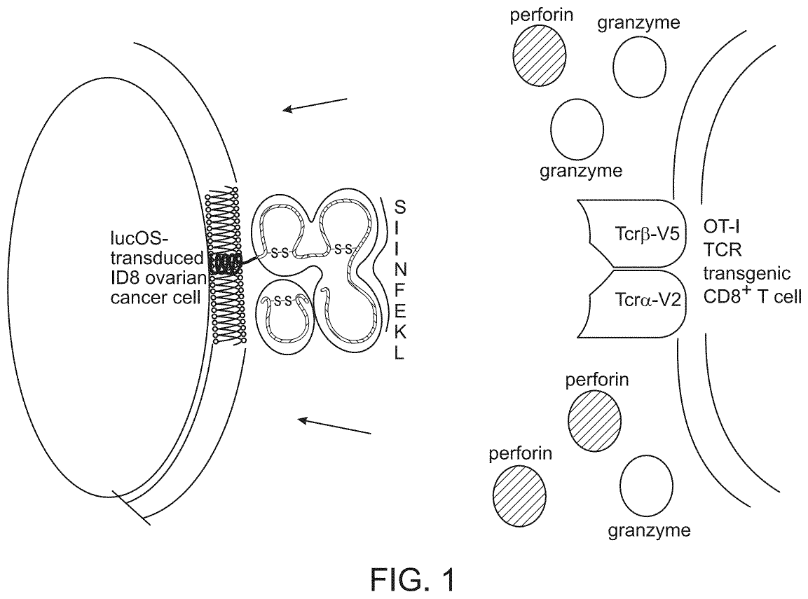

CTL-mediated killing of target cells that present MHC-I antigens is performed by CD8⁺ T cells, which release perforin and granzyme to achieve killing of target cells (FIG. 1). OT-I CTL cells (Ovalbumin-specific CD8⁺ T cell receptor transgenic line OT-I cytotoxic T lymphocyte), which are CD8⁺ T cells that specifically recognize and kill ovalbumin-presenting target cells, were obtained and used to target the above-described ID8 cells expressing firefly luciferase as a reporter peptide and chicken ovalbumin as a MHC-I antigen (FIG. 1).

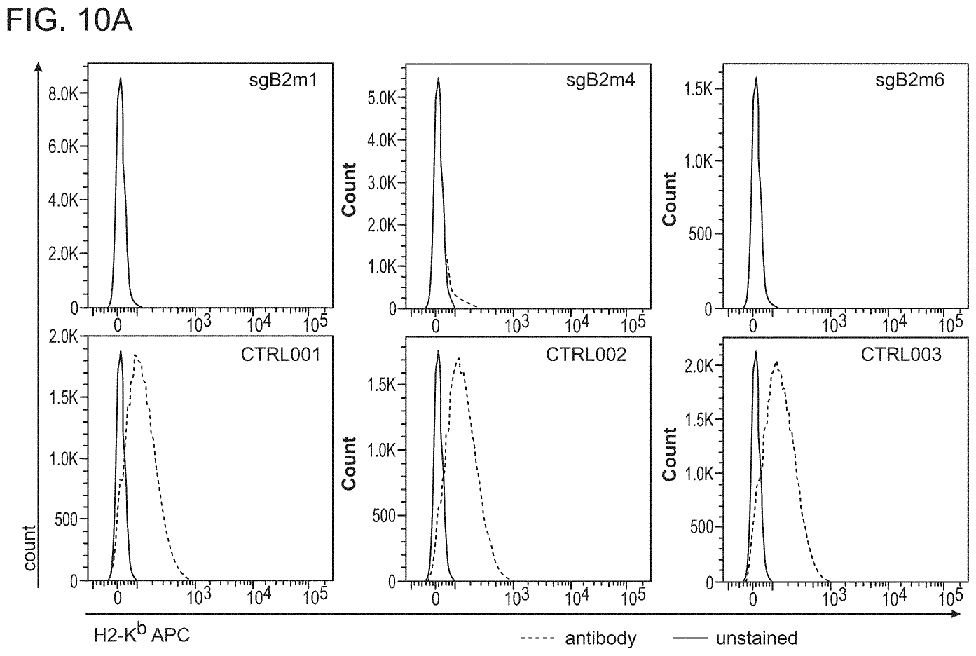

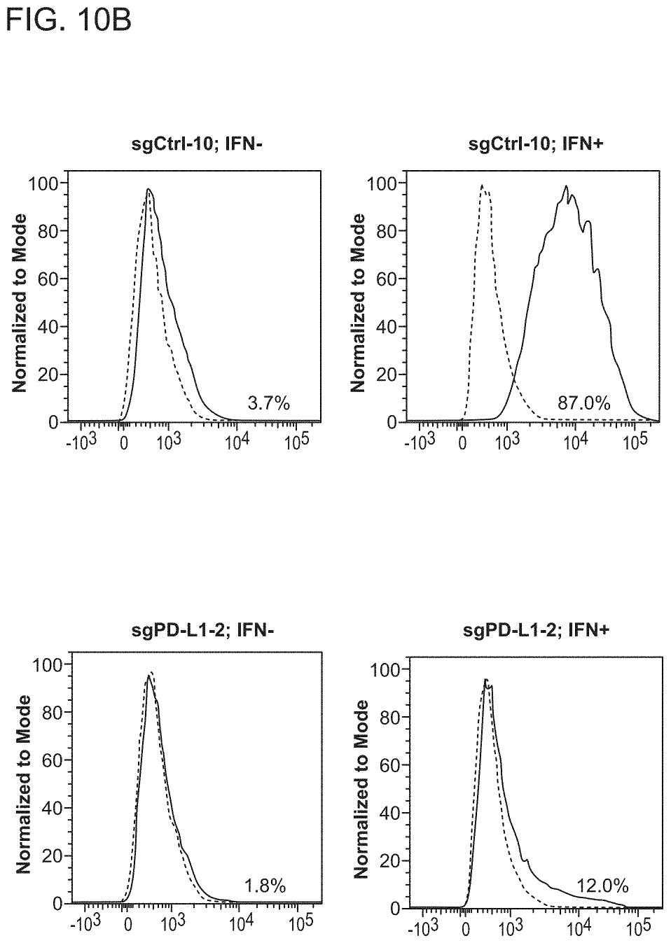

To identify immunomodulatory compounds capable of enhancing or disrupting MHC-I-specific interactions between a CD8⁺ T cell and a target cell that presents a MHC-I-displayed antigen, a cell-based test system was developed. For development of the OT-I IO assay, the ID8 murine serous ovarian carcinoma cell line was utilized due to its constitutive expression of MHC class-I, MHC haplotype compatibility with C57BL/6J mice, and dramatic IFN-γ-induced upregulation of PD-L1 (FIG. 10A and FIG. 10B). ID8s were transduced with pLVX vectors to express either firefly luciferase and OVA model antigen (“lucOS”) or renilla luciferase and no model antigen (“rluc”) (FIG. 2A). Target ID8 cell lines were mixed at a 1:1 ratio and co-cultured with CD8+ T cells isolated from the spleens of OT-I TCR-transgenic mice; OT-I mice express transgenic TCRα-V2 and TCRβ-V5 genes such that all CD8+ T cell receptors recognize chicken ovalbumin residues 257-264 (SIINFEKL) in the context of H-2Kb (Hogquist et al., 1994 Cell, 76:17-27). Target cell-T cell cultures were incubated with compounds for 48 hr and then analyzed by dual-luciferase assay whereby changes in firefly signal relative to controls indicated modulation of T cell killing by compound treatment (FIG. 2B and FIG. 2C).

As shown in FIG. 1 and FIG. 2A, cells of a highly proliferative mouse ovarian cancer cell line, ID8—specifically a Cas9-expressing “clone A10” of ID8, were virally transduced with one of two constructs: (1) a DNA construct harboring firefly luciferase as a first reporter peptide operably linked to chicken ovalbumin as a model antigen peptide, further including a Neomycin cassette for selection purposes (FIG. 2A) or (2) a control DNA construct harboring renilla luciferase as a second reporter peptide, lacking model antigen peptide, but further including a Neomycin cassette for selection purposes (FIG. 2A). Notably, ID8 cells transduced with the DNA construct harboring firefly luciferase as a first reporter peptide operably linked to chicken ovalbumin as a model antigen peptide were selected for (via G418 selection limiting dilution) and confirmed both to express firefly luciferase and to present ovalbumin as a MHC-I antigen at the cell surface (FIG. 1; ID8-lucOS “clone B9” cells). Control ID8 cells transduced with the DNA construct harboring renilla luciferase as a second reporter peptide were also selected for (via G418 selection limiting dilution) and confirmed to express renilla luciferase (FIG. 1; ID8-rluc “clone C3” cells).

A high-throughput assay capable of identifying test compounds that specifically impaired or enhanced CD8⁺ T cell-mediated killing of target ID8-lucOS “clone B9” cells in an antigen-specific manner was developed, as depicted in FIG. 2B. In such assays, a 96-well tissue culture plate array format was employed, and in each well, 10,000 ID8-lucOS and 10,000 ID8-rluc were co-plated. OT-I TCR transgenic CD8⁺ T cells were then plated on top of ID8 cells in each well, and these transgenic CD8⁺ T cells were observed to selectively kill ID8-lucOS in an antigen-dependent manner, while sparing ID8-rluc cells. The OT-I assay was then performed as a high-throughput screen (FIG. 2C) to evaluate test compounds for immunomodulatory effects upon antigen-specific tumor cell killing by cytotoxic T lymphocytes (CTLs). Inclusion of ID8-lucOS and ID8-rluc in each well of the array-formatted test compound assay provided in-plate normalization controls, which allowed for identification of test compounds that effected non-specific growth inhibition or induction of apoptosis, versus identification of modulation by screen compounds of antigen-specific tumor cell killing by cytotoxic T lymphocytes. Such high-throughput screening was performed under standard ID8 cell growth conditions (37° C. and 5% 02) for initial rounds of test compound screening. As shown in FIG. 2C, additional rounds of test compound screening can be performed under a number of other conditions, including, e.g., in the presence of hypoxia, hydrogen peroxide (H₂O₂), TGF-β/IL-10, T regulatory cells (Tregs), myeloid-derived suppressor cells (MDSCs), in the absence of L-arginine and/or L-cysteine, etc.

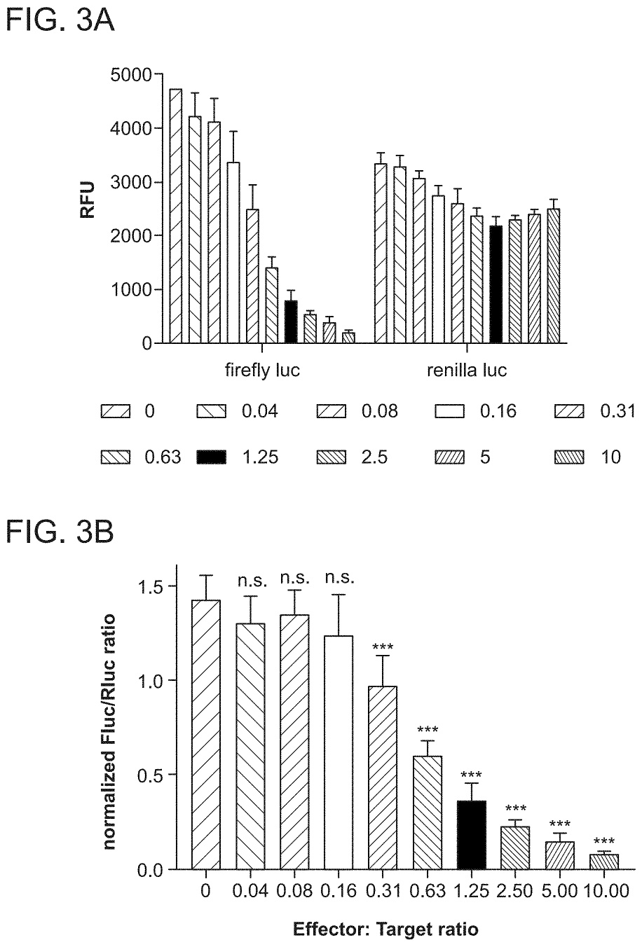

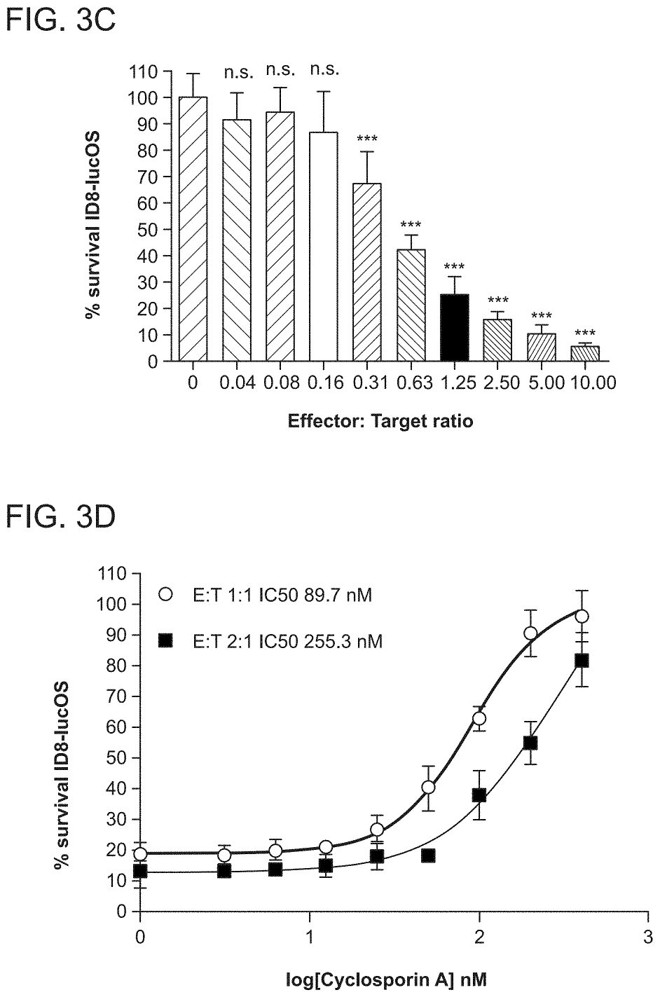

Renilla luciferase signal remains relatively constant across wells regardless of the number of OT-I T cells added to co-culture. However, there was a dramatic loss of firefly luciferase signal with increasing Effector:Target ratios, indicating that OT-I CD8+ T cells selectively kill lucOS ID8 cells in an antigen-dependent manner, while sparing rluc ID8 cells (FIG. 3A). Simple calculation of Fluc/Rluc ratio or of % surviving OVA-expressing target cells ([Fluc+OT-I/Rluc+OT-I]/[avg Fluc no OT-I/avg Rluc no OT-I]×100) revealed that an Effector:Target ratio of ˜0.5 was sufficient to observe ˜50% killing (FIG. 3B and FIG. 3C). The OT-I IO assay was validated with cyclosporin-A, a well-established inhibitor of CD8+ T cell effector function (Schulz et al., 2004 Dev Biol, 266:1-16). As expected, cyclosporin-A inhibited T cell-mediated killing of antigen-expressing target cells in a dose-dependent manner, consistent with published IC₅₀ values (FIG. 3D).

Specifically, dose-responsiveness of ID8-lucOS cells to administration of varying levels of OT-I TCR transgenic CD8⁺ T cells was initially assessed to validate overall functioning of the OT-I assay. As shown in FIG. 3A, firefly luciferase levels (fluc was expressed by ID-8 ovarian cancer cells also expressing ovalbumin as a model antigen peptide) declined upon administration of increasing numbers of OT-I CD8⁺ T cells, whereas rluc levels showed no statistically significant disparities across varying concentrations of OT-I CD8⁺ T cells (increasing effector cell:target cell ratios), consistent with the OT-I CD8⁺ T cells targeting ovalbumin-expressing target cells in a specific manner, thereby establishing the viability of using the OT-I assay to screen test compounds for modulation of the MHC-I-specific OT-I CD8⁺ T cell-mediated killing of ID8-lucOS cells. Similar levels of dose-response to OT-I CD8⁺ T cells were also observed via normalization of firefly luciferase levels to renilla luciferase levels (FIG. 3B) or by calculating % survival of target ID8-lucOS cells (FIG. 3C). Each showed significant antigen-specific tumor cell killing with effector cell:target cell ratios as low as 0.31 and approximately 50% killing at an effector cell:target cell ratio of 1. Administration of cyclosporin A, which is an inhibitor of calcineurin and a well-characterized inhibitor of CD8⁺ T cell effector function, was also confirmed as capable of reversing the impact of adding CD8⁺ T cells to the target ID8-lucOS cell-containing population (FIG. 3D). Cyclosporin A was therefore used as a control compound to validate assay performance, and SHP1/2 inhibitor controls were also spiked into the OT-I compound screening assays. Each screening plate had ID8-only controls for assessing non-specific growth inhibitory effects and ID8+OT-I T cells, and all plates were run in duplicate (total of 16 96-well plates). Edge wells were excluded during screening assay performance, and multiple DMSO-only control wells were present on each plate, to allow for sufficiently robust appropriate control values. Compounds were incubated for 48 hours, and a dual-glo luciferase assay was employed to detect both firefly and renilla luciferase levels (also distinguishing between the two).

The OT-I assay was thereby preliminarily validated as a platform for identification of immunomodulatory test compounds specific for modulation of CD8⁺ T cell effector function.

### Example 3: OT-I Assay Identified Immunomodulatory Lead Compounds

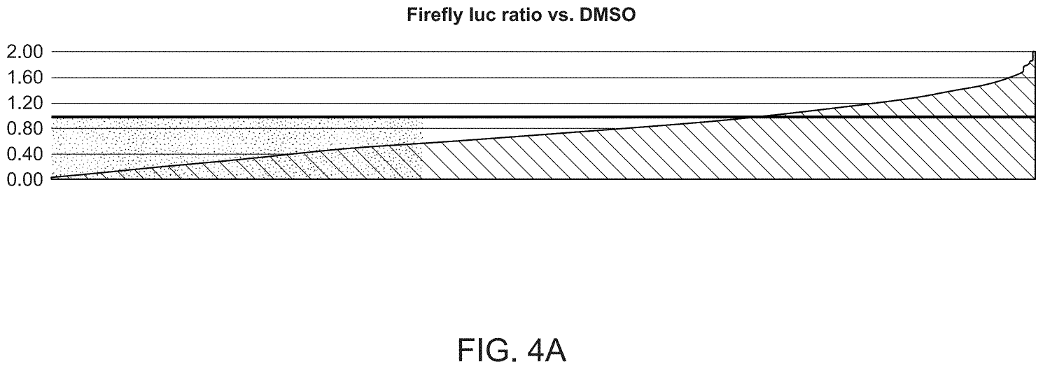

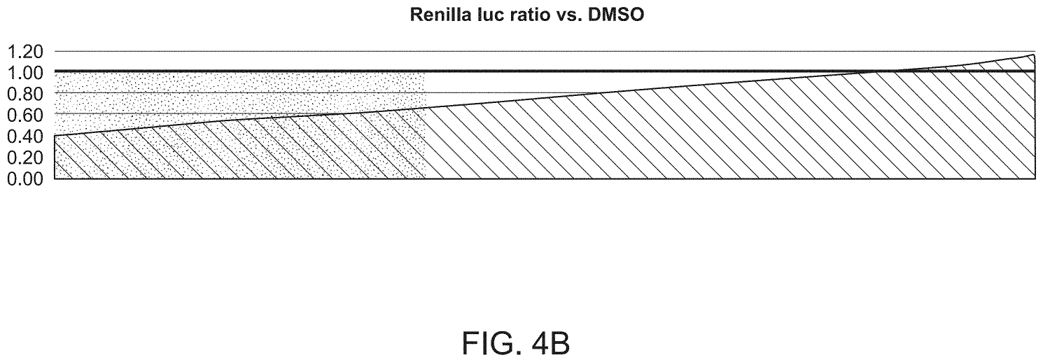

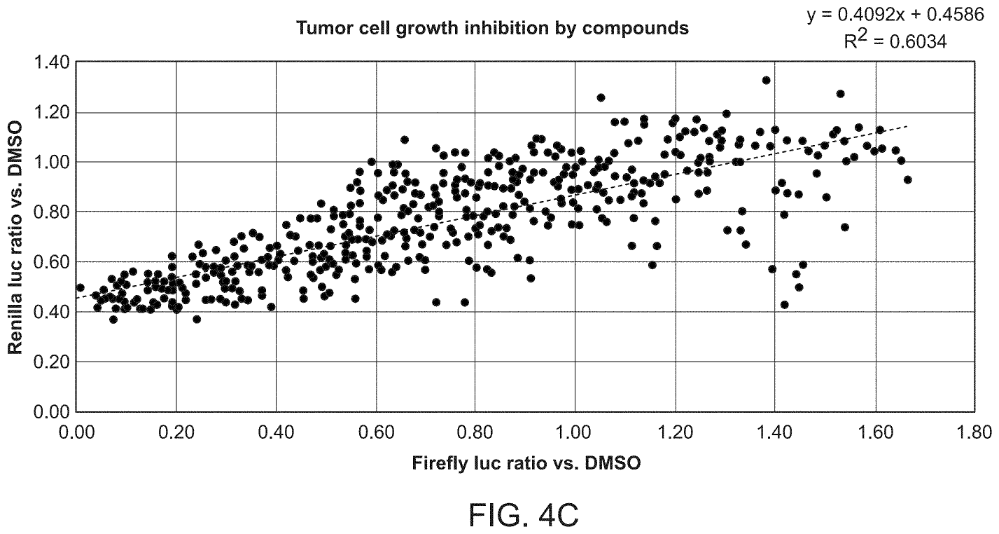

For OT-I IO assay pilot screen and hit validation, the OT-I IO assay was screened with a focused library of kinase inhibitors from the Harvard Medical School NIH LINCS Center (provided above). Compounds were screened at a 1 μM concentration, a dose at which nearly a third of the compounds caused non-specific loss of viability in both antigen-expressing lucOS and control rluc ID8 cells; these compounds were removed from further analysis (FIG. 4A-FIG. 4C).

Specifically, 203 test compounds were selected and administered in the high-throughput format. Control plates in which test compounds were administered in the absence of OT-I T cells were initially examined. As shown in FIG. 4A-FIG. 4C, DMSO control wells were identified as results that should have been consistent across all assays, because DMSO treatment should not have affected ID8 tumor cell viability. Accordingly, raw luciferase values were initially normalized relative to the DMSO average, and it was predicted that such normalization should have provided a distribution with most compounds exhibiting values around 1 (that don't affect growth) and some fraction above (that augment growth) or below 1 (that inhibit growth). This initial normalization of compound-treated ID8 cell luciferase results thereby revealed many compounds as non-specifically inhibiting ID8 cell growth, thereby also underscoring the need for inclusion of rluc-expressing ID8 cells as control cells within the OT-I assays of the current disclosure.

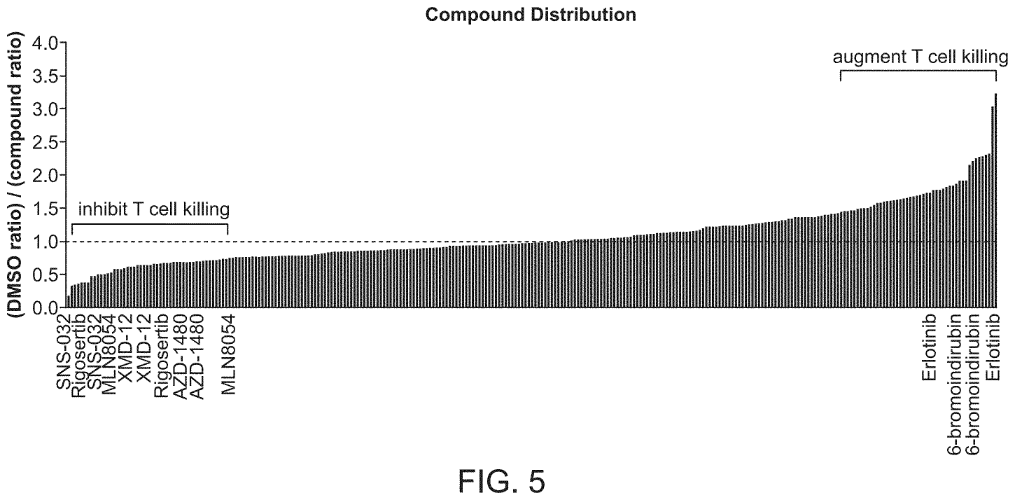

Screen results were analyzed based on normalized firefly/renilla luciferase ratio in DMSO control wells relative to compound-treated wells (FIG. 5). Compounds were considered hits if they fell in the top or bottom 10% of compounds and scored in all replicate plates. Compounds inhibiting OT-I T cell killing have ratios <1, inert compounds have ratios ˜1, and compounds that augment T cell killing display ratios >1. The CDK9 inhibitor SNS-032, PLK1 inhibitor Rigosertib, Aurora kinase A inhibitor MLN8054, JAK2 inhibitor AZD-1480, and Aurora kinase inhibitor XMD-12-1 (Kwiatkowski et al., 2012 ACS Chem Biol, 7:185-96; Miduturu et al., 2011 Chem Biol, 18:868-79) all inhibited T cell-mediated target cell lysis (FIG. 5). The GSK-3β inhibitor 6-bromoindirubin and EGFR inhibitor erlotinib were the only two compounds that significantly augmented T cell killing.

Specifically, the charts of FIG. 5 further depict that many test compounds inhibited tumor cell growth and/or killed tumor cells, when administered in the absence of OT-I cells. Notably, firefly luciferase values, even for DMSO-only wells, produced a much greater spread of DMSO-normalized values than renilla luciferase values, which tended to be much more consistent. Blank plate runs also resulted in differences of a few hundred units from well to well, so at the low end of the range, the assay was thereby identified as exhibiting low sensitivity.

Where both OT-I T cells and test compounds were administered, normalized firefly/renilla luciferase ratios relative to DMSO-only control wells were assessed and calculated for each test compound (FIG. 5). Compounds that inhibited OT-I T cell killing exhibited ratios <1 (JAK2 inhibitor, CDK9 inhibitor, PLK1 inhibitor), while compounds identified as inert exhibited ratios of approximately 1, and compounds that augmented T cell killing displayed ratios >1 (e.g., EGFR inhibitors). Plates were screened in duplicate, and compounds were considered “hits” only if they scored in both plates.

### Example 4: Validation of OT-1 Assay-Identified Immunomodulatory Lead Compounds

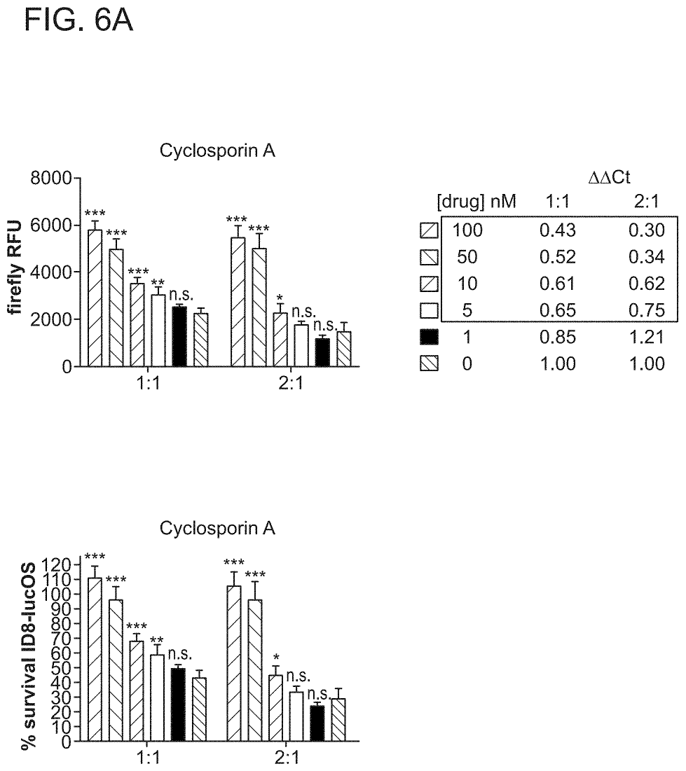

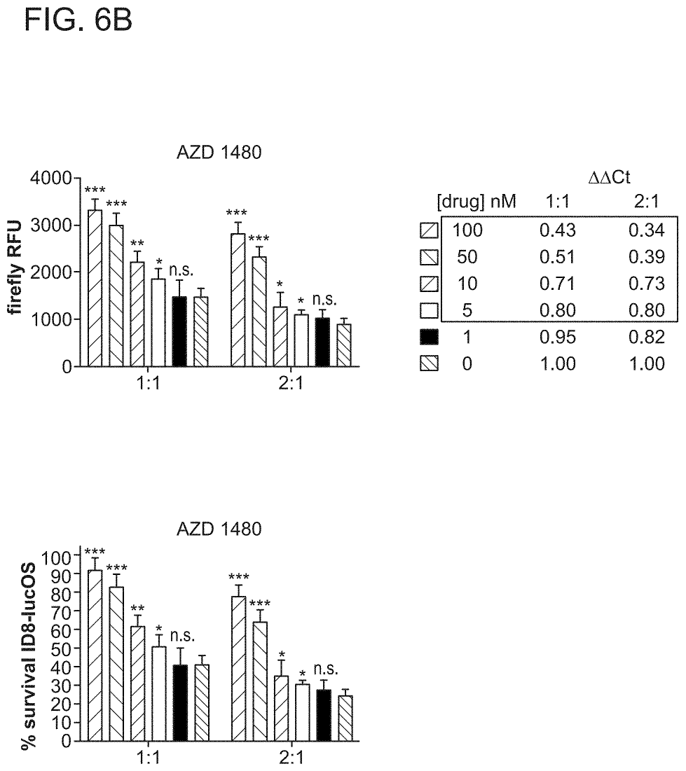

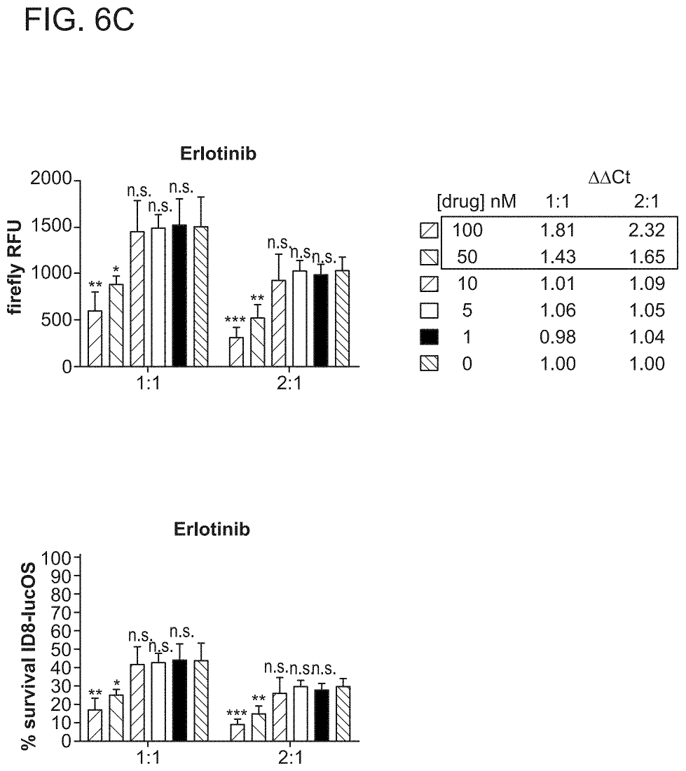

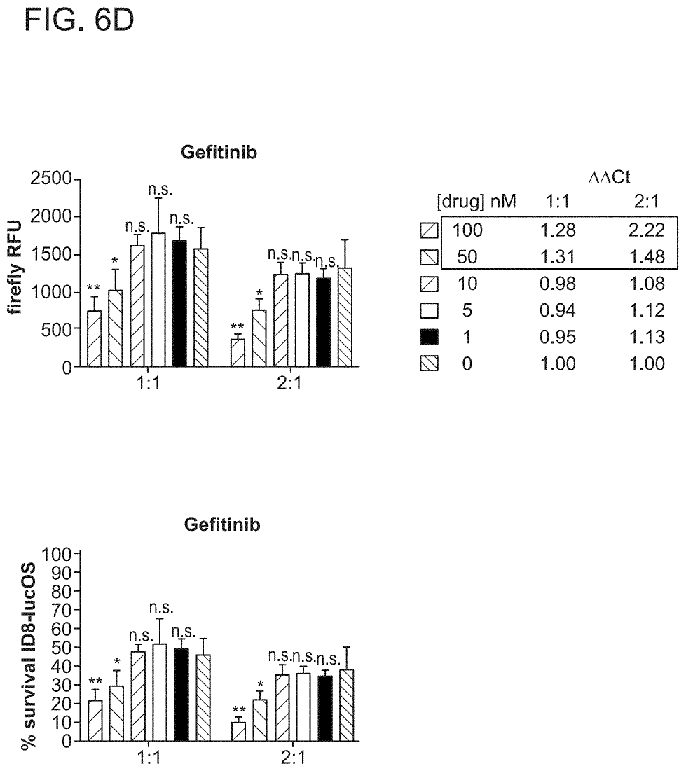

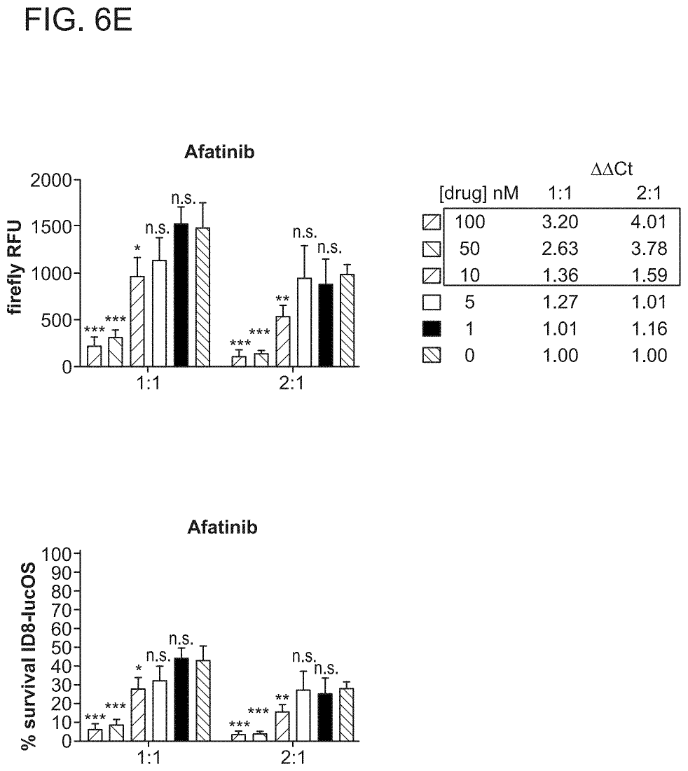

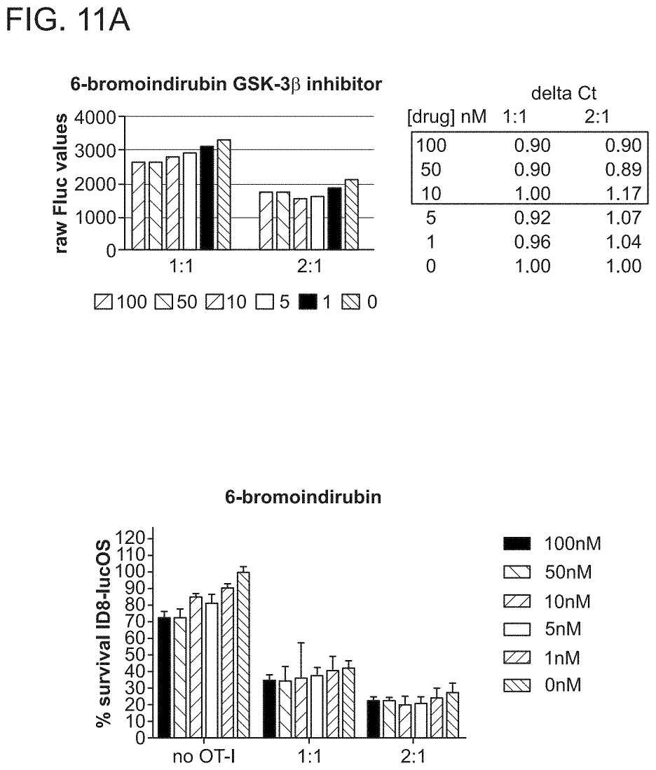

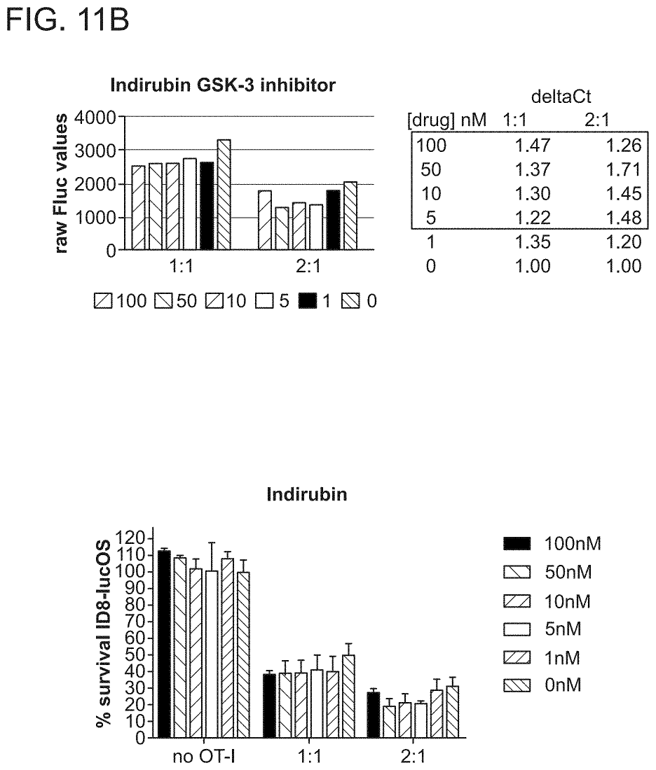

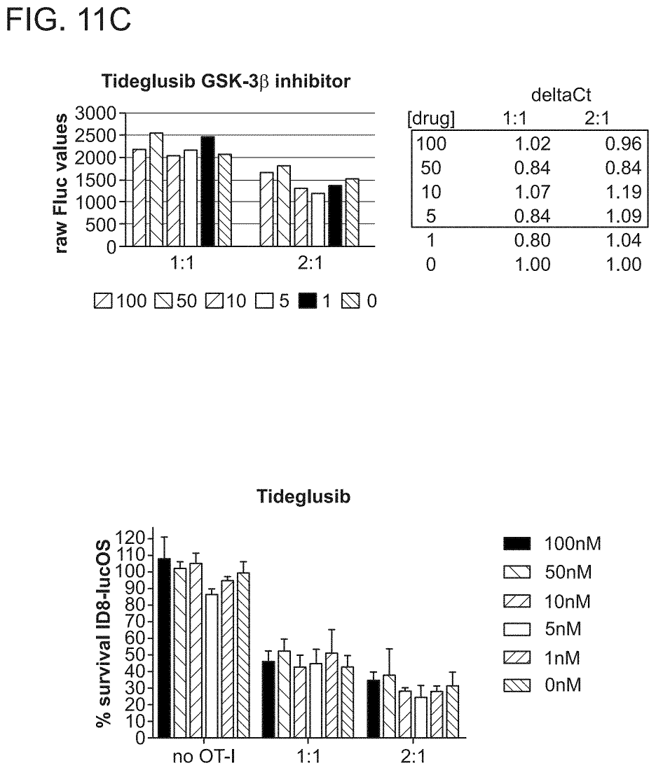

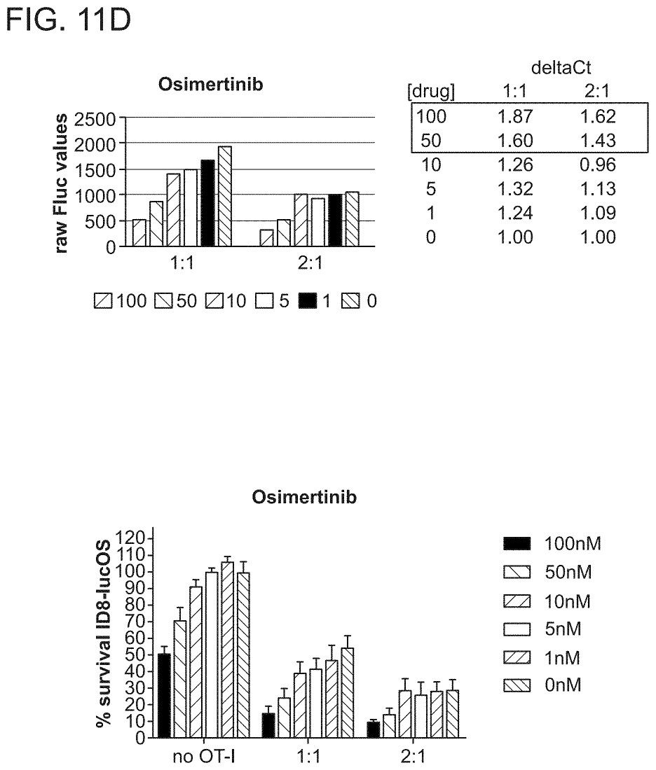

Hits from the initial larger compound screen were validated in the OT-I IO assay with dose-response curves. The JAK2 inhibitor AZD-1480 significantly inhibited T cell killing with similar kinetics to cyclosporin-A, down to low nM concentrations (FIG. 6A and FIG. 6B). Of more interest for synergistic application with immune checkpoint blockade, however, were compounds that augmented T cell killing. Upon further testing, the GSK-3β inhibitor 6-bromoindirubin and other GSK-3β-specific and GSK-3 inhibitors only modestly augmented T cell killing (FIG. 11A-FIG. 11D). The EGFR inhibitor erlotinib, however, was confirmed to augment T cell-mediated tumor cell lysis (FIG. 6C). To determine if this effect was erlotinib-specific or EGFR-specific, gefitinib, an alternative EGFR-specific ATP competitive inhibitor, and afatinib, an irreversible EGFR inhibitor of different chemotype, were also examined. All three EGFR inhibitors significantly augmented OT-I T cell killing and, in the case of afatinib, resulted in lysis of almost all OVA-expressing ID8 target cells even at concentrations down to 10 nM (FIG. 6C, FIG. 6D, and FIG. 6E). The T790M-specific EGFR tyrosine kinase inhibitor (TKI), osimertinib, exhibited modestly enhanced killing that was inferior to erlotinib, gefitinib, and afatinib (Supp. FIG. 3; RENUMBER). Osimertinib has activity against wtEGFR at high concentrations (Cross et al., 2014 Cancer Discov, 4:1046-61).

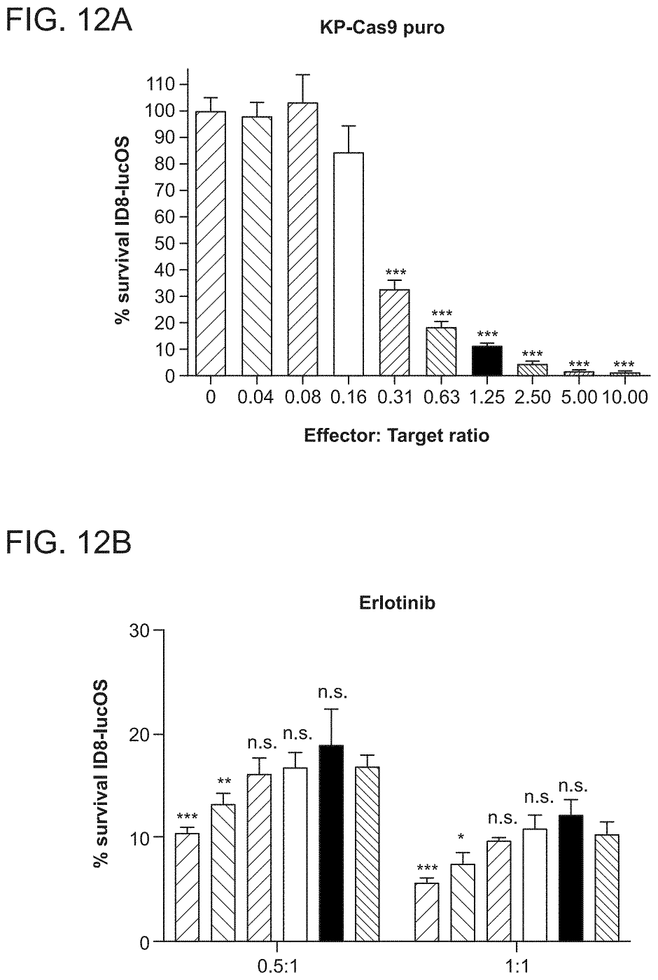

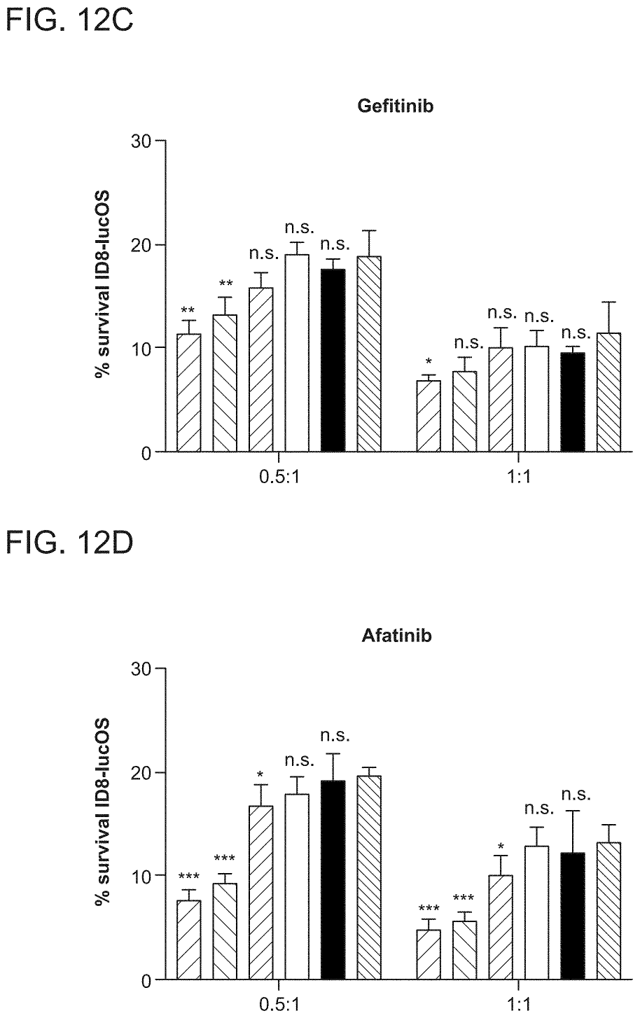

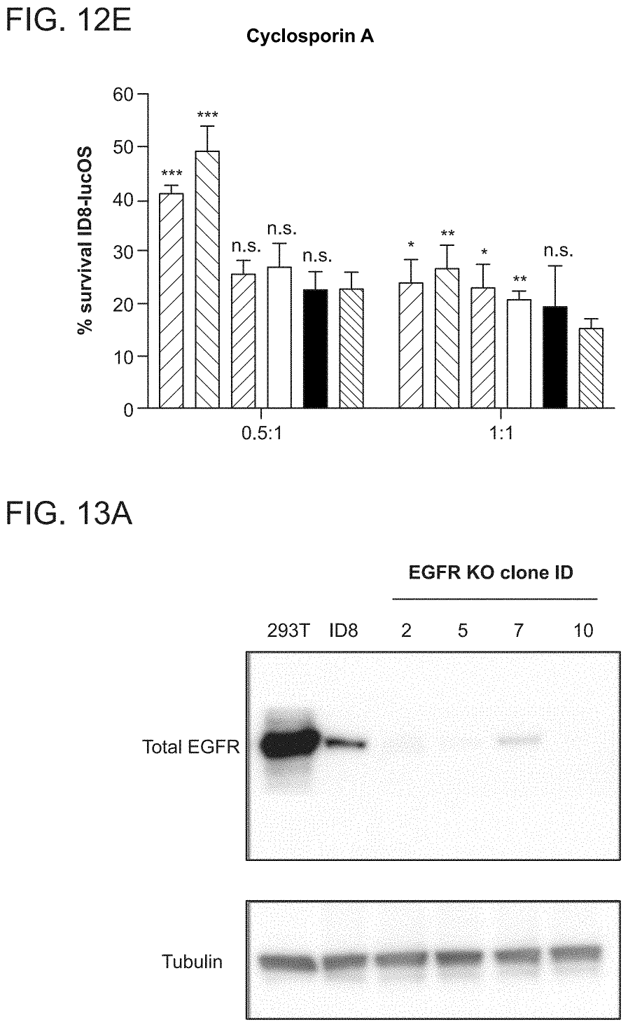

To eliminate the possibility that the EGFR sensitivity observed in the assay was an artifact of the ID8 cell line, the CTL assay was also performed in a cell line derived from the Kras^{G12D}/p53^{-/-} C57BL/6J lung adenocarcinoma model (FIG. 12A-FIG. 12E; Pollack et al., 2011 Clin Cancer Res, 17:4400-13). The KP cell line was transduced with the same vectors to stably express Cas9 and the lusOS construct and co-cultured with OT-I CD8+ T cells. OT-I T cell-mediated lysis of OVA-expressing KP cells was significantly enhanced by EGFR inhibitors erlotinib, Gefinitib, and afatinib and inhibited by cyclosporin-A, further confirming the initial ID8 screen result (FIG. 12A-FIG. 12E).

As described above, among the initial screening assay hits, the JAK2 inhibitor, AZD-1480, was identified as a specific inhibitor of T cell killing of ID8 target cells, whereas the EGFR inhibitor, erlotinib, was identified as a specific enhancer of T cell killing of ID8 target cells. To confirm that such test compounds initially identified in the high-throughput OT-I assay as capable of modulating CD8⁺ T cell-mediated killing of target ID8-lucOS “clone B9” cells in an antigen-specific manner validated as immunomodulatory, the dose-responsiveness of these test compounds' effects, as well as other EGFR inhibitors, was assessed on a compound-by-compound basis. As shown in FIG. 6A, the control compound, cyclosporin A, exhibited a predicted, dose-responsive inhibition of OT-I T cell-mediated killing (increasing amounts of cyclosporin A maintained firefly luciferase levels by blocking CD8⁺ T cell-mediated killing of ovalbumin-expressing cells). Meanwhile, as show in FIG. 6B, AZD 1480 (a JAK2 inhibitor), which was the top hit of the 203 test compound screen for inhibition of T cell-mediated killing, performed similarly to cyclosporin A, which thereby supported the assessment from the larger compound screen that AZD 1480 could also disrupt CD8⁺ T cell-mediated killing of ovalbumin-expressing cells, as was demonstrated for AZD 1480 across a broader dose range (thereby verifying the similar dose-responsiveness of the observed effect).

Further testing of EGFR inhibitors revealed that erlotinib (FIG. 6C), which was identified as the top hit of the 203 test compound screen for augmenting T cell-mediated killing, as well as two other EGFR inhibitors, gefitinib (FIG. 6D) and afatinib (FIG. 6E) impacted CD8⁺ T cell-mediated killing in a dose-responsive manner, at least at higher test compound concentrations (increasing levels of the EGFR inhibitors increased T cell-mediated killing in the screening assay). Inhibition of EGFR with any of these test compounds therefore augmented antigen-specific CD8⁺ T cell-mediated killing.

### Tumor Cell-Intrinsic Effect of EGFR Inhibition

Cell culture media from the OT-I IO assay was harvested from wells following 48 hr of culture with a range of EGFR inhibitors and the JAK2 inhibitor AZD-1480 and assessed for IFN-γ levels by ELISA. Secretion of IFN-γ was used as a proxy for OT-I T cell effector function. As expected, escalating doses of AZD-1480 significantly inhibited IFN-γ secretion in a dose-dependent manner (FIG. 7A). None of the EGFR inhibitors affected IFN-γ secretion, leading to the conclusion that the immunomodulatory effect of EGFR inhibition in the assay was not due to T cell-intrinsic effects. The highest concentration of EGFR inhibitors, 100 nM, modestly reduced IFN-γ secretion, likely due to T cell toxicity.

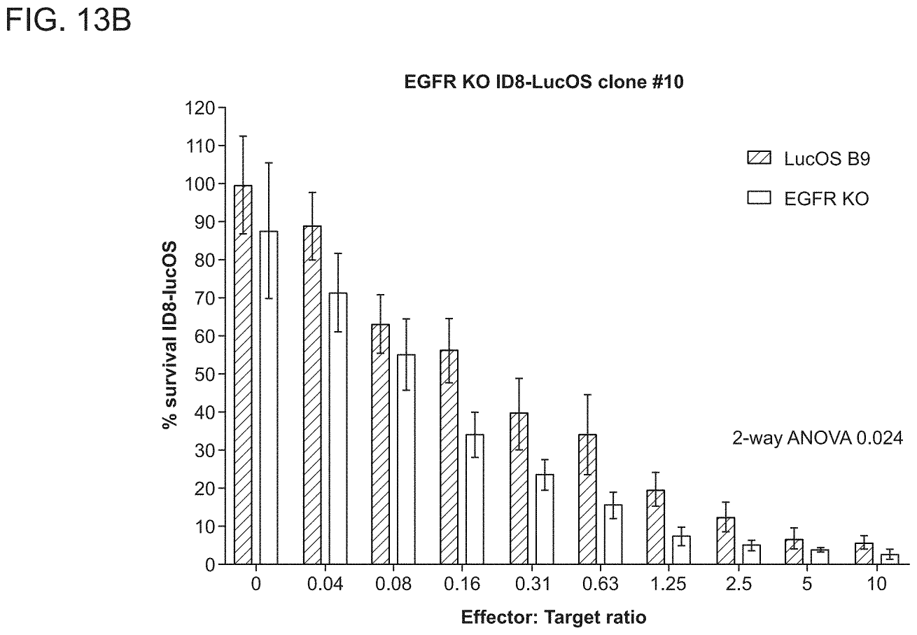

EGFR inhibitors increase basal and IFN-γ-induced expression of MHC class-I expression in human keratinocytes (Pollack et al., 2011 Clin Cancer Res, 17:4400-13), leading to the investigation of whether this mechanism might explain the increased T cell-mediated killing following treatment with EGFR inhibitors in the assay. It was observed that erlotinib, gefitinib, and afatinib all significantly increased both basal expression of MHC class-I by ID8 tumor cells and MHC class-I expression induced by physiological levels of IFN-γ (FIG. 7B). EGFR inhibitor-induced upregulation of MHC class-I expression also correlated with performance of the varying EGFR inhibitors in the OT-I IO assay; the irreversible inhibitor afatinib was superior to ATP competitive inhibitors erlotinib and gefitinib. The same cell line transduced with multiple different sgRNA targeting EGFR (FIG. 13A and FIG. 13B) also exhibited increased basal and IFN-γ-induced expression of MHC class-I (FIG. 7C). Additionally, Kras^{G12D};p53^{-/-} lung adenocarcinoma (FIG. 7D) and MC38 colon cancer (FIG. 7E) cell lines responded to EGFR inhibitor treatment by significantly increasing surface MHC class-I.

As described above, the impact of EGFR inhibitors was further validated via assessment of ELISA interferon gamma (IFNγ) levels. As shown in FIG. 7A, EGFR inhibition enhanced T cell killing via what appeared to be a tumor cell intrinsic mechanism, whereas the AZD 1480 compound—which was newly identified as an inhibitor of T cell-mediated killing of target cells—significantly decreased T cell IFN-γ production in a dose-dependent manner. The ELISA results of FIG. 7A specifically revealed that all EGFR inhibitors tested (erlotinib, gefitinib and afatanib) did not appear to exert any enhancing effect upon OT-I CD8⁺ T cell IFN-γ secretion, as might have been predicted if the EGFR inhibitors were simply exerting an effect upon OT-I CD8⁺ T cells that was effectively the opposite of the effect that the T cell inhibitory AZD 1480 compound was observed to exert. Without wishing to be bound by theory, because none of the EGFR inhibitors exhibited dose-responsiveness that would indicate enhancement of IFN-γ production as correspondingly enhancing the T cell-mediated killing of target cells that was observed at rising EGFR inhibitor concentrations, it was surmised that the EGFR inhibitors were exerting their effect upon the tumor cells, rather than upon the OT-I CD8⁺ T cells, possibly by making the tumor cells (target cells) more susceptible to T cell-mediated killing. None of the EGFR inhibitors affected IFNn-γ secretion, leading to the conclusion that the immunomodulatory effect of EGFR inhibition in this assay was not due to T cell-intrinsic effects. The highest concentration of EGFR inhibitors, 100 nM, modestly reduced IFN-γ secretion, likely due to T cell toxicity.

Without wishing to be bound by theory, it was believed that a potential mechanism of action for the effects observed for EGFR inhibitors involved increased antigen processing and presentation by MHC class I. In particular, it was previously reported that EGFR inhibitors increase basal and IFN-γ-induced expression of MHC class-I expression in human keratinocytes (Pollack et al. Clin. Cancer Res. 17, 4400-4413), and it was therefore investigated whether this mechanism could explain the increased T cell-mediated killing observed in the instant assays after treatment with EGFR inhibitors. As shown in FIG. 7B, where (erlotinib, gefitinib and afatanib, were administered for 48h at 100 ng/mL) relative to control (DMSO) treatments, erlotinib, gefitinib, and afatinib all significantly increased both basal expression of MHC class-I by ID8 tumor cells and MHC class-I expression induced by physiological levels of IFN-γ (4 pg/mL; FIG. 7B). EGFR inhibitor-induced upregulation in MHC class-I expression also correlated with performance of the varying EGFR inhibitors in the OT-I IO (immune-oncology) assay: the irreversible inhibitor afatrinib was superior to ATP competitive inhibitors erlotinib and gefitinib. The same cell line transduced with multiple different sgRNA targeting EGFR (FIG. 13A and FIG. 13B) also exhibited increased basal and IFN-γ-induced expression of MHC class-I (FIG. 7C). Additionally, Kras^{G12D};p53^{-/-} lung adenocarcinoma (FIG. 7D) and MC38 colon cancer (FIG. 7E) cell lines responded to EGFR inhibitor treatment by significantly increasing surface MHC class-I.

EGFR Inhibitors Synergize with Anti-PD-1 Therapy

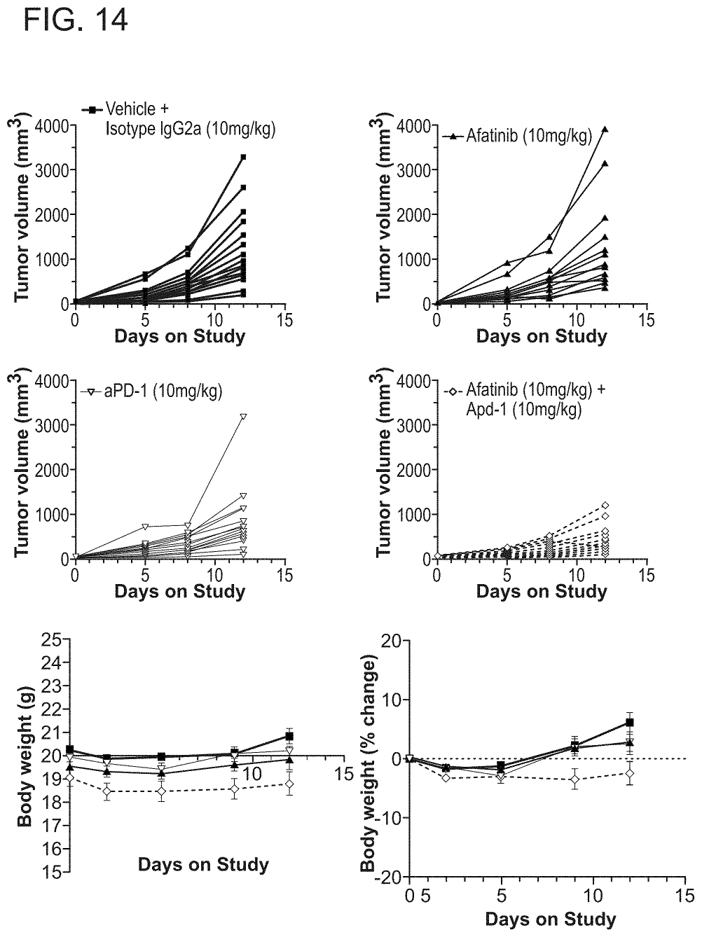

The high antigenicity of the ID8-lucOS model and diffuse nature of the etiology precluded the use of these cells for in vivo validation. Instead, the syngeneic MC38 colon was utilized due to its well-established, moderate sensitivity to immune checkpoint blockade (Deng et al., 2014 J Clin Invest, 124:687-95; Ngiow et al., 2015 Cancer Res, 75:3800-11; Zippelius et al., 2015 Cancer Immunol Res, 3:236-44). Mice were implanted with MC38 tumor and then treated with vehicle+isotype control, afatinib, anti-PD-1, or combination afatinib+anti-PD-1. Combination EGFR inhibition and PD-1 blockade significantly delayed tumor progression relative to vehicle+isotype control, afatinib, and anti-PD-1 (FIG. 8A, FIG. 8B, and FIG. 8C). Combination therapy also conferred significantly improved survival relative to controls (p=0.003) while anti-PD-1 conferred modest (p=0.026), and afatinib single agent none (p=0.487) (FIG. 8D). Combination afatinib and anti-PD-1 was highly consistent in its tumor inhibition across all 15 mice and dosing was well-tolerated (FIG. 14). Additionally, therapeutic efficacy of the combination treatment was lost when CD8+ T cells were depleted, confirming that the effect was immune-mediated. It was concluded that combination PD-1 blockade and EGFR pharmacological inhibition constitutes a synergistic immunotherapy.

As described above, in vivo efficacy of the EGFR inhibitors was also assessed, alone or in combination with PD-1 blocking agents. As shown in FIG. 8A to FIG. 8D, EGFR inhibition enhanced in vivo efficacy of PD-1 blockade. Specifically, combination EGFR inhibition and PD-1 blockade significantly delayed tumor progression relative to vehicle+isotype control, afatinib, and anti-PD-1 (FIG. 8A-FIG. 8C). Mice receiving combination treatment of anti-PD-1 and the EGFR inhibitor, afatinib, exhibited significantly reduced tumor burden on day 12 (see, FIG. 8A and FIG. 8B). Further, as shown in FIG. 8C, mice receiving combination treatment of anti-PD-1 and the EGFR inhibitor, afatinib, exhibited significantly inhibited tumor growth kinetics. Meanwhile, mice receiving combination treatment of anti-PD-1 and the EGFR inhibitor afatinib exhibited significantly improved survival relative to other treatments (FIG. 8D). Specifically, combination therapy conferred significantly improved survival relative to controls (p=0.003), while anti-PD-1 conferred modest (p=0.026), and afatinib single agent none (p=0.487) (FIG. 8D). Combination afatinib and anti-PD-1 was highly consistent in its tumor inhibition across all 15 mice and dosing was well-tolerated (FIG. 14). Accordingly, combination PD-1 blockade and EGFR pharmacological inhibition constitutes a synergistic immunotherapy.

The high antigenicity of the ID8-lucOS model and diffuse nature of the etiology precluded the use of these cells for in vivo validation. Instead, the syngeneic MC38 colon was utilized due to its well-established, moderate sensitivity to immune checkpoint blockade (Deng, et al. 2014 J Clin Invest, 124:687-95; Ngiow, et al., 2015 Cancer Res, 75:3800-11; and Zippelius et al., 2015 Cancer Immunol Res, 236-44). Mice were implanted with MC38 tumor and then treated with vehicle+isotype control, afatinib, anti-PD-1, or combination afatinib+anti-PD-1. Specifically, for each of the experiments, C57BL/6J mice were challenged subcutaneously with 500,000 MC38 colon cancer cells on their flanks and “enrolled” on-study when tumors reached 50 mm³. Mice were treated with aPD-1 (anti-PD1) on days 5, 8, and 12 and afatinib on days 6, 7, 8, 9, and 10 (where indicated). Flank tumor growth curves were analyzed using two-way ANOVA, bar graphs were analyzed using unpaired Student's t-test, and survival experiments used the log-rank Mantel-Cox test for survival analysis, all indicated with *p<0.05; **p<0.01; ***p<0.001.

Additional assessments of EGFR inhibitors can also be performed, alone or in combination with PD-1/PD-L1 blocking agents and/or antibodies that block CTLA-4, BTLA, VISTA, B7-H3, KIR, TIGIT, TIM-3 or LAG-3, or antibodies or other agents that act as agonists to 4-1BB, OX40, CD40/CD40L, ICOS, GITR or CD28. Combination therapies are expected to further enhance T cell-mediated killing of target cells.

Also, a retrospective cohort was compiled of 41 relapsed/metastatic squamous cell carcinoma of the head and neck (r/m SCCHN) patients who received combination afatinib and the anti-PD-1 antibody pembrolizumab (FIG. 8E and FIG. 8F) at the National Taiwan University Hospital between November 2016 and September 2017. Combination therapy resulted in a ORR of 58.5% by RECIST criteria and an average tumor size reduction of 82.9%, and without associated increased toxicity (FIG. 8E, FIG. 8F, and FIG. 8G). This is compared to reported ORR of 16% to pembrolizumab monotherapy (Larkin et al., 2015 N Engl J Med, 373:23-34) and ORR of 10% to afatinib monotherapy (Manguso et al., 2017 Nature, 547:413-8) in r/m SCCHN patients. The analysis demonstrated clear translational therapeutic impact for r/m SCCHN patients treated with combination EGFR inhibitor afatinib and pembrolizumab PD-1 blockade.

### Example 5: CRISPR Library OT-I Assay Screen for Immunomodulatory Lead Agents

As described herein, a CRISPR/Cas9 screen independently identifies EGFR as immunomodulatory. The OVA-expressing ID8 target cell line was also engineered to constitutively expresses the Cas9 gene, enabling the transduction of these cells with an sgRNA library and perform the OT-I 10 assay in pooled format. A library of ˜8,000 sgRNAs comprised of 87 control genes (essential genes, oncogenes, tumor suppressor genes), 86 immune modulators (immune checkpoints, differentially regulated immune genes), 524 epigenetic regulators, and 34 MHC genes at a coverage of 10 sgRNA per gene was utilized, and also included 500 non-targeting sgRNA. ID8 lucOS cells were transduced with the lentiviral library and cultured at a representation of 500 cells/sgRNA for 72 hr in the presence or absence of OT-I effector CD8+ T cells. In the absence of OT-I T cells, as expected, sgRNAs targeting essential genes were preferentially depleted in surviving cells (FIG. 9A, FIG. 9B, and FIG. 9C, red bars). With the addition of OT-I T cells, it was expected that positive control sgRNAs that targeted immunosuppressive mechanisms, such as PD-L1, would enhance CTL killing, while negative control sgRNAs targeting MHC class-I processing and presentation gene should inhibit CTL killing. sgRNAs targeting H2-K1, Tap1, Tap2, and B2m scored as four of the top seven genes enriched in live cells following co-culture with OT-I CTLs (FIG. 9E, green bars). sgRNAs targeting the positive control, PD-L1, were preferentially depleted in live cells, indicating that loss of this immunosuppressive surface receptor sensitized the ID8 cells to T cell-mediated killing (FIG. 9F, green bar). Intriguingly, sgRNAs targeting EGFR were preferentially depleted from surviving ID8 cells, indicating that loss of EGFR sensitized tumor cells to T cell-mediate killing; in fact, EGFR scored as #10 out of 731 genes depleted in live cells (FIG. 9F, FIG. 9G, and FIG. 9H). Top ranking sgRNAs targeting EGFR were used to make individual stable EGFR KO cell lines, which were also sensitized to OT-I T cell-mediated killing across a wide range of Effector:Target ratios, validating the pooled CRISPR screen result (FIG. 13A and FIG. 13B).

ID8 target cells constitutively expressing Cas9 were employed in the above-described OT-I assay. The current OT-I assay format therefore allowed for CRISPR agents to be screened for immunomodulatory character. To identify immunomodulatory agents/genetic targets via a CRISPR/Cas9 screening approach, OVA-expressing ID8 target cells were transduced with a sgRNA library, and the OT-I 10 assay was performed upon such cells in pooled format. A library of ˜8,000 sgRNAs was employed, which was comprised of 87 control genes (essential genes, oncogenes, tumor suppressor genes), 86 immune modulators (immune checkpoints, differentially regulated immune genes), 524 epigenetic regulators, and 34 MHC genes, at a coverage of 10 sgRNA per gene, and the library also included 500 non-targeting sgRNA. ID8 lucOS cells were transduced with the lentiviral library and cultured at a representation of 500 cells/sgRNA for 72 hr in the presence or absence of OT-I effector CD8+ T cells. As assessed for test compounds of the above-described OT-I assay screen, CRISPR agents (sgRNAs) that displayed immunomodulatory effects (i.e. preferential survival or preferential apoptosis relative to non-targeting sgRNA) in the high-throughput OT-I assay were thereby identified.

The distribution of assayed sgRNA representation levels in live versus dead cells in the absence of OT-I CD8⁺ T cells was initially assessed, with results shown in FIG. 9A. Representation data for the ten sgRNAs that exhibited the greatest enrichment in live cells (versus dead cells) was identified and plotted (FIG. 9B), as was representation data for the ten sgRNAs that exhibited the greatest depletion in live cells (versus dead cells; FIG. 9C). As expected, in the absence of OT-I T cells, sgRNAs targeting essential genes were preferentially depleted in surviving cells (FIG. 9A to FIG. 9C, noting shaded bars of non-control samples in FIG. 9C). Upon addition of OT-I T cells, it was expected that positive control sgRNA that target immunosuppressive mechanisms, such as PD-L1, would enhance CTL killing, whereas negative control sgRNA targeting MHC class-I processing and presentation would be expected to inhibit CTL killing. As shown in FIG. 9E, sgRNA targeting H2-K1, Tap1, Tap2, and B2m scored as four of the top seven genes enriched in live cells following co-culture with OT-I CTLs (FIG. 9E, shaded bars of non-control genes). sgRNA targeting the positive control, PD-L1, were preferentially depleted in live cells, indicating that loss of this immunosuppressive surface receptor sensitized the ID8 cells to T cell-mediated killing (FIG. 9F to FIG. 9H, noting Egfr results and dots as dark blue shaded (non-black)). Intriguingly, sgRNA targeting Egfr were preferentially depleted from surviving ID8 cells; in fact, Egfr scored as #10 out of 731 genes depleted in live cells (FIG. 9F).

Thus, it was demonstrated that CRISPR/Cas9 screening data independently arrived at identification that inhibition of EGFR augmented anti-tumor immunity. The above-described compound screen and genetic screen were both unbiased (among distinct compounds/sgRNAs screened) and identified the same target (Egfr).

The above sgRNA-based screen revealed B2m sgRNAs (among others, including H2-K1, Hdac8, Tap1, Ep300, Tap2, Cbx5, Brwd1, Cbx3 and Chrac1) as also capable of inhibiting CD8⁺ T cell killing of target cells (FIG. 10A). As shown in FIG. 10A and FIG. 10B, Cas9 was confirmed as active in the ID8 cells, and these cells were confirmed to respond to IFN-γ by upregulating PD-L1. This responsiveness was therefore shown to be successfully prevented by transducing the cells with sgRNAs targeting the PD-L1 gene (FIG. 10B).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for identifying an agent capable of modulating the interaction between a CD8⁺ T cell and a cell expressing a model antigen peptide, comprising:
contacting a first population of cells comprising a mixture of (1) cells expressing a model antigen peptide and a first reporter peptide and (2) cells that express a second reporter peptide and do not express the model antigen peptide, with a test agent;
assessing expression of the first reporter peptide, the second reporter peptide, or both the first and second reporter peptides, in the first cell population, as compared to an appropriate control cell population expressing the reporter peptide(s) and not contacted with the test agent;
contacting a second population of cells comprising a mixture of (1) CD8⁺ T cells; (2) cells expressing the model antigen peptide and the first reporter peptide; and (3) cells that express the second reporter peptide and do not express the model antigen peptide, with the test agent;
assessing expression of the first and second reporter peptides in the second cell population, as compared to an appropriate control cell population not contacted with the test agent and expressing the first and second reporter peptides, and
identifying the test agent as an agent that modulates CD8⁺ T cell killing of the cells expressing the model antigen peptide if the test agent:
(a) is not identified to modulate expression of the first reporter peptide, the second reporter peptide, or both the first and second reporter peptides in the first cell population, as compared to the appropriate control cell population expressing the reporter peptide(s) and not contacted with the test agent; and
(b) is identified to significantly increase or significantly decrease expression of the first reporter peptide relative to the second reporter peptide in the second population of cells, as compared to the appropriate control cell population not contacted with the test agent and expressing the first and second reporter peptides,
thereby identifying the test agent as an agent capable of modulating the interaction between a CD8⁺ T cell and a cell expressing a model antigen peptide.

2. The method of claim 1, wherein the cell expressing a model antigen peptide is an ovarian cancer cell.

3. The method of claim 2, wherein the ovarian cancer cell comprises a nucleotide sequence encoding for the model antigen peptide, operably linked to nucleotide sequence encoding for the first reporter peptide.

4. The method of claim 1, wherein the (1) cells expressing a model antigen peptide and a first reporter peptide and (2) cells that express a second reporter peptide and do not express the model antigen peptide, are derived from the same source cell line, optionally wherein the source cell line is an ovarian cancer cell line, optionally ID8 cells.

5. The method of claim 1, wherein the CD8⁺ T cell is an OT-I T cell receptor transgenic cell.

6. The method of claim 1, wherein the first population of cells, the second population of cells, or both the first and second populations of cells are in an array, optionally in a 96 well plate format.

7. The method of claim 1, wherein in the first population of cells, there is an about 1:1 proportion of (1) cells expressing a model antigen peptide and a first reporter peptide to (2) cells that express a second reporter peptide and do not express the model antigen peptide.

8. The method of claim 1, wherein in the second population of cells, there is at least about a 2:10 proportion of (1) CD8⁺ T cells to (2) cells expressing the model antigen peptide and the first reporter peptide, optionally about a 3:10 to about a 10:1 proportion of (1) CD8⁺ T cells to (2) cells expressing the model antigen peptide and the first reporter peptide, optionally about a 1:1 to about a 2:1 proportion of (1) CD8⁺ T cells to (2) cells expressing the model antigen peptide and the first reporter peptide.

9. The method of claim 1, wherein the first reporter peptide or the second reporter peptide is firefly luciferase; or
wherein the second reporter peptide or the first reporter peptide is renilla luciferase; or
wherein the first reporter peptide is firefly luciferase and the second reporter peptide is renilla luciferase.

10.-11. (canceled)

12. The method of claim 1, wherein the test agent is identified as an agent that modulates the viability of the first population of cells if the expression of the reporter peptide(s) is significantly increased or significantly reduced in the first population of cells, as compared to an appropriate control cell population.

13. The method of claim 12, wherein the test agent is identified as an agent that reduces the viability of the first population of cells if the expression of the reporter peptide(s) is reduced by at least about two-fold in the first population of cells, as compared to an appropriate control cell population, optionally wherein the appropriate control cell population is a cell population not contacted with a test agent, optionally wherein the appropriate control cell population is contacted with DMSO.

14. The method of claim 1, wherein the first population of cells and the second population of cells are contacted under standard cell growth conditions, optionally at 37° C. and 5% 02.

15. The method of claim 1, wherein the first population of cells and the second population of cells are grown and/or contacted under one or more of the following conditions: hypoxic conditions, in the presence of hydrogen peroxide, in the presence of TGF-β and/or IL-10, in the presence of T regulatory cells, in the presence of MDSCs (myeloid-derived suppressor cells), in the absence of L-arginine and/or in the absence of L-cysteine.

16. The method of claim 1, wherein at least one of the assessing steps is performed at between 12 h and 72 h after the first population of cells or the second population of cells is contacted with test agent, optionally wherein the at least one of the assessing steps is performed at about 48 h after the first population of cells or the second population of cells is contacted with test agent, optionally wherein the assessing steps are performed at about 48 h after the first population of cells is contacted with test agent and at about 48 h after the second population of cells is contacted with test agent, respectively.

17. The method of claim 1, wherein the test agent is a small molecule; or
wherein the test agent is selected from the group consisting of Seliciclib ((R)-Roscovitine; CYC202; target=CDK2); ALW-II-38-3 (target=DDR1); ALW-II-49-7 (target=DDR1); AT-7519 (target=CDK9); Tivozanib (AV-951; target=VEGFR-2); AZD7762 (target=CHK1); AZD8055 (target=mTOR); Sorafenib (BAY-439006; target=BRAF); CP466722 (target=ATM); CP724714 (target=erbB-2); Alvocidib (Flavopiridol; HMR-1275; L868275; target=CDK1); GSK429286A (target=ROCK1); GSK461364 (GSK461364A; target=PLK1); GW843682X (GW843682; target=PLK1); HG-5-113-01 (target=LOK); HG-5-88-01 (target=EGFR); HG-6-64-01 (KIN001-206; target=ABL1); Neratinib (HKI-272; target=erbB-2); JW-7-24-1 (target=LCK); Dasatinib (BMS-354825; Sprycel; target=ABL1); Tozasertib (VX680; MK-0457; target=Aurora kinase A); GNF2 (target=ABL1); Imatinib (Gleevec; Glivec; CGP-57148B; STI-571; target=ABL1); NVP-TAE684 (TAE-684; target=ALK); CGP60474 (MLS000911536; SMR000463552; target=CDK1); PD173074 (target=FGFR1); Crizotinib (PF02341066; target=c-Met); BMS345541 (target=IKKB); LY2090314 KIN001-042 (target=GSK-3 beta); KIN001-043 (target=GSK-3 beta); Saracatinib (AZD0530; target=Src); KIN001-055 (target=JAK3); AS601245 (JNK Inhibitor V; target=JNK3); Sigma A6730KIN001-102; AKT inhibitor VIII; Akt1/2 kinase inhibitor (target=Akt-1); SB 239063 (target=MK14); AC220 (target=FLT3); WH-4-023 (target=LCK); R406 (target=SYK); BI-2536 (NPK33-1-98-1; target=PLK1); Motesanib (AMG706; target=VGFR1); KIN001-127 (target=ITK); A443654 (target=Akt-1); SB590885 (target=BRAF); Pictilisib (Pictrelisib; GDC-0941; RG-7321; target=PIK3CA); PD184352 (CI-1040; target=MP2K1); PLX-4720 (target=BRAF); AZ-628 (target=BRAF); Lapatinib (GW-572016; Tykerb; target=EGFR); Sirolimus (Rapamycin; target=mTOR); ZSTK474 (target=PIK3CA); AS605240 (target=PIK3CG); BX-912 (target=PDK1); Selumetinib (AZD6244; Arrayl42886; target=MP2K1); MK2206 (target=Akt-1); CG-930 (JNK930; target=JNK1); AZD-6482 (KIN001-193; target=PIK3CB); TAK-715 (target=MK14); NU7441 (KU 57788; target=DNA-PK); GSK1070916 (KIN001-216; target=Aurora kinase B); OSI-027 WYE-125132 (target=mTOR); KIN001-220 (Genentech 10; target=Aurora kinase A); MLN8054 (target=Aurora kinase A); Barasertib (AZD1152-HQPA; target=Aurora kinase B); Vemurafenib (PLX4032; RG7204; R7204; R05185426; target=BRAF); Enzastaurin (LY317615; target=KPCB); NPK76-II-72-1 (target=PLK3); Palbociclib (PD0332991; target=CDK4); PF562271 (KIN001-205; target=FAK); PHA-793887 (target=CDK2); KU55933 (target=ATM); QL-X-138 (target=BTK); QL-XI-92 (target=DDR1); QL-XII-47 (target=BTK); THZ-2-98-01 (target=IRAK1); Torin1 (target=mTOR); Torin2 (target=mTOR); KIN001-244 (target=PDK1); WZ-4-145 (target=CSF1R); WZ-7043 (target=CSF1R); WZ3105 (target=CLK2); WZ4002 (target=EGFR); XMD11-50 (LRRK2-in-1; target=LRRK2); XMD11-85h (target=BRSK2); XMD13-2 (target=RIPK1); XMD14-99 (target=EPHB3); XMD15-27 (target=CAMK2B); XMD16-144 (target=Aurora kinase A); JWE-035 (target=Aurora kinase A); XMD8-85 (target=ERK5); XMD8-92 (target=ERK5); ZG-10 (target=JNK1); ZM-447439 (target=Aurora kinase A); Erlotinib (OSI-774; target=EGFR); Gefitinib (ZD1839; Iressa; target=EGFR); Nilotinib (AMN-107; target=ABL1); JNK-9L (KIN001-204; target=JNK1); PD0325901 (PD-325901; target=MP2K1); MPS-1-IN-1 (HG-5-125-01); XMD-12 YM 201636 (Kin001-170; target=FYV1); FR180204 (FR 180204; KIN001-230; target=ERK-1); TWS119 (target=GSK-3 beta); PF477736 (target=CHK1); Kin237 (Kin001-237; c-Met/Ron dual kinase inhibitor; target=c-Met); Pazopanib (GW786034; Votrient); LDN-193189 (DM 3189; target=ACVR1); PF431396 (target=FAK); Celastrol (target=PSB5); Amuvatinib (MP470; target=PGFRA); SU11274 (PKI-SU11274; target=c-Met); Canertinib (CI-1033; PD-183805; target=EGFR); SB525334 (target=TGFR1); NVP-AEW541 (AEW541; target=IGF1R); SGX523 (target=c-Met); MGCD265 (target=c-Met); PHA-665752 (target=c-Met); PI103 (target=PIK3CA); Dovitinib (TKI_258; TKI258; target=FLT3); GSK 690693 (target=Akt-1); Ibrutinib (PCI-32765; target=BTK); Masitinib (AB1010; target=c-Kit); Tivantinib (ARQ197; target=c-Met); SNS-032 (BMS-387032; target=CDK9); Afatinib (BIBW-2992; target=erbB-2); GSK1904529A (target=IGF1R); Linsitinib (OSI 906; target=IGF1R); TPCA-1 (target=IKKB); BMS509744 (BMS-509744; target=ITK); Ruxolitinib AZD-1480 (target=JAK2); Momelotinib (CYT387; target=JAK1); Fedratinib (SAR 302503; SAR-302503; SAR302503; TG 101348; Tg-101348; TG101348; target=JAK2); Trametinib (GSK-1120212; GSK1120212; GSK1120212B; JTP-74057; target=MP2K1); BMS 777607 (target=c-Met); Olaparib (AZD2281; KU-0059436; target=PARP-1); Veliparib (ABT-888; target=PARP-1); Omipalisib (GSK2126458; GSK2126458A; target=PIK3CA); Buparlisib (BKM120; NVP-BKM120; target=PIK3CA); XL147 (SAR245408; target=PIK3CA); Y39983 (target=ROCK1); Ponatinib (AP24534; target=ABL1); Nintedanib (BIBF-1120; Vargatef; target=VGFR1); MK 1775 (target=WEE1hu); KIN001-266 (target=M3K8); AT7867 (target=Akt-1); KU-60019 (target=ATM); JNJ38877605 (target=c-Met); Foretinib (XL880; GSK1363089; target=c-Met); AZD 5438 (KIN001-239; target=CDK2); Pelitinib (EKB-569; target=EGFR); SB 216763 (target=GSK-3 beta); Luminespib (NVP-AUY922; target=HS90A); SP600125 (target=JNK1); BIX 02189 (target=MP2K5); AZD8330 (ARRY-424704; ARRY-704; target=MP2K1); PF04217903 (target=c-Met); BAY61-3606 (target=SYK); SB 203580 (RWJ 64809; PB 203580; target=MK14); VX-745 (target=MK14); Doramapimod (BIRB 796; target=MK14); JNJ 26854165 (target=p53); TGX221 (target=PIK3CB); GSK1059615 (target=PIK3CA); PI3K-IN-1 (target=mTOR); A 769662 (target=AMPK-alpha1); Sunitinib (Sutent; SU11248); Y-27632 (target=ROCK1); Brivanib (BMS-540215; target=VGFR1); OSI-930 (target=c-Kit); ABT-737 (target=BCL2); CHIR-99021 (CT99021; KIN001-157; target=GSK-3 beta); GDC-0879 (target=BRAF); Linifanib (ABT-869; AL-39324; target=FLT3); BGJ398 (KIN001-271; NVP-BGJ398; target=FGFR1); Rigosertib (ON-01910; target=PLK1); CC-401 (target=JNK1); Chelerythrine (target=KPCB); Ki20227 (target=CSF1R); BX795 (target=TBK1); Bosutinib (SKI-606; target=Src); PIK-93 (target=PIK3CG); HMN-214 (target=PLK1); KW2449 (KW-2449; target=FLT3); Kin236 (Tie2 kinase inhibitor; target=TIE2); Cabozantinib (XL-184; BMS-907351; target=VEGFR-2); KIN001-269 (target=CSF1R); KIN001-270 (target=CDK9); KIN001-260 (IKK-2 inhibitor VIII; Bayer IKKb inhibitor; target=IKKB); Vandetanib (ZD6474; Zactima; Caprelsa; target=VEGFR-2); PF 573228 (target=FAK); NVP-BHG712 (KIN001-265; target=EPHB4); CH5424802 (target=ALK); D 4476 (target=TGFR1); A66 (target=PIK3CA); CAL-101 (target=PIK3CD); INK-128 (MLN0128; target=mTOR); RAF 265 (CHIR-265; target=BRAF); NVP-TAE226 (target=FAK); and JNK-IN-5A (TCS JNK 5a; KIN001-188; target=MK09); or
wherein the test agent is a CRISPR agent.

18.-19. (canceled)

20. The method of claim 1, wherein the test agent is identified to enhance CD8⁺ T cell killing of the cells expressing the model antigen peptide; or
wherein the test agent is identified to inhibit CD8⁺ T cell killing of the cells expressing the model antigen peptide.

21. (canceled)

22. A cell mixture comprising:
(a) a first population of cells comprising a nucleotide sequence encoding for a model antigen peptide, operably linked to nucleotide sequence encoding for a first reporter peptide; and
(b) a second population of cells comprising a nucleotide sequence encoding for a second reporter peptide.

23. The cell mixture of claim 22, wherein the first population of cells comprising a nucleotide sequence encoding for a model antigen peptide is an ovarian cancer cell population.

24. The cell mixture of claim 22, wherein the (1) first population of cells comprising a nucleotide sequence encoding for a model antigen peptide, operably linked to nucleotide sequence encoding for a first reporter peptide and the (2) second population of cells comprising a nucleotide sequence encoding for a second reporter peptide, are derived from the same source cell line, optionally wherein the source cell line is a carcinoma cell line, optionally an ovarian carcinoma cell line, optionally ID8 cells.

25. The cell mixture of claim 22, further comprising a third population of cells that is a CD8⁺ T cell population, optionally wherein the third population of cells that is a CD8⁺ T cell population present in at least about a 2:10 proportion to the first population of cells comprising the nucleotide sequence encoding for the model antigen peptide, optionally wherein the third population of cells that is a CD8⁺ T cell population present in about a 3:10 to about a 10:1 proportion to the first population of cells comprising the nucleotide sequence encoding for the model antigen peptide, optionally wherein the third population of cells that is a CD8⁺ T cell population present in about a 1:1 to about a 2:1 proportion to the first population of cells comprising the nucleotide sequence encoding for the model antigen peptide.

26. The cell mixture of claim 25, wherein the third population of cells that is a CD8⁺ T cell population is an OT-I T cell receptor transgenic cell population.

27. The cell mixture of claim 22, wherein the cell mixture is present in an array, optionally in a 96 well plate format.

28. The cell mixture of claim 22, comprising an about 1:1 proportion of (1) the first population of cells comprising a nucleotide sequence encoding for a model antigen peptide, operably linked to nucleotide sequence encoding for a first reporter peptide and (2) the second population of cells comprising a nucleotide sequence encoding for a second reporter peptide.

29. The cell mixture of claim 22, wherein the first reporter peptide or the second reporter peptide is firefly luciferase; or
wherein the second reporter peptide or the first reporter peptide is renilla luciferase; or
wherein the first reporter peptide is firefly luciferase and the second reporter peptide is renilla luciferase.

30.-31. (canceled)

32. The cell mixture of claim 22, wherein the first population of cells is an immortalized cell line; or
wherein the first reporter peptide is a luciferase peptide, optionally firefly luciferase.

33. (canceled)

34. The cell mixture of claim 32, wherein the second reporter peptide is a luciferase peptide distinct from the first reporter peptide, optionally wherein the second reporter peptide is renilla luciferase.

35. A method for enhancing CD8⁺ T cell killing of target cells in a subject, comprising:
administering a pharmaceutical composition comprising an EGFR inhibitor and a pharmaceutically acceptable carrier to the subject in an amount sufficient to enhance CD8⁺ T cell killing of target cells in the subject; or
a method for inhibiting CD8⁺ T cell killing of target cells in a subject, comprising:
administering a pharmaceutical composition comprising a JAK2 inhibitor and a pharmaceutically acceptable carrier to the subject in an amount sufficient to inhibit CD8⁺ T cell killing of target cells in the subject; or
a method for enhancing CD8⁺ T cell killing of target cells in a subject, comprising:
administering a pharmaceutical composition comprising a Noc4I inhibitor, a Prpf19 inhibitor, a Prmt5 inhibitor, a Fbxw7 inhibitor, an Eif3a inhibitor, a Cd274 inhibitor, a Mta2 inhibitor, a Natl 0 inhibitor and/or a Map3k7 inhibitor and a pharmaceutically acceptable carrier to the subject in an amount sufficient to enhance CD8⁺ T cell killing of target cells in the subject; or
a method for inhibiting CD8⁺ T cell killing of target cells in a subject, comprising:
administering a pharmaceutical composition comprising a H2-K1 inhibitor, a Hdac8 inhibitor, a Tap1 inhibitor, an Ep300 inhibitor, a Tap2 inhibitor, a Cbx5 inhibitor, a B2m inhibitor, a Brwd1 inhibitor, a Cbx3 inhibitor and/or a Chrac1 inhibitor and a pharmaceutically acceptable carrier to the subject in an amount sufficient to inhibit CD8⁺ T cell killing of target cells in the subject.

36. The method of claim 35, wherein the target cells are selected from the group consisting of ovarian cancer cells, lung cancer cells, colorectal cancer cells, glioblastoma cells, breast cancer cells, prostate cancer cells, renal cancer cells, melanoma and pancreatic cancer cells.

37. The method of claim 35, wherein the subject is human; or
wherein the subject is murine.

38. (canceled)

39. The method of claim 35, wherein the target cells are cells of a cancer cell line, optionally an ovarian cancer cell line, optionally ID8 cells.

40. The method of claim 35, wherein the EGFR inhibitor is selected from the group consisting of erlotinib, gefitinib, afatinib and osimertinib.

41.-42. (canceled)

43. The method of claim 35, wherein the subject is human; or wherein the subject is murine.

44.-45. (canceled)

46. The method of claim 35, wherein the JAK2 inhibitor is selected from the group consisting of AZD-1480, Pacritinib, Gandotinib, XL019, BMS-911543, AZ 960, Fedratinib, NVP-BSK805 2HCl and CEP-33779.

47. A method for treating or preventing a neoplasia in a subject, comprising:
administering a pharmaceutical composition comprising:
(i) an EGFR inhibitor;
(ii) an anti-PD-1 agent, an anti-CTLA agent, an anti-KIR agent, an anti-TIGIT agent, an anti-TIM-3 agent, an anti-LAG-3 agent, a 4-1BB agonist, an ICOS agonist, a GITR agonist or a CD28 agonist; and
(iii) a pharmaceutically acceptable carrier
to the subject in an amount sufficient to treat or prevent the neoplasia in the subject; or
a method for treating or preventing a neoplasia in a subject, comprising:
administering a pharmaceutical composition comprising:
(i) a Noc4I inhibitor, a Prpf19 inhibitor, a Prmt5 inhibitor, a Fbxw7 inhibitor, an Eif3a inhibitor, a Cd274 inhibitor, a Mta2 inhibitor, a Nat10 inhibitor and/or a Map3k7 inhibitor;
(ii) an anti-PD-1/PD-L1 agent, an anti-CTLA agent, an anti-KIR agent, an anti-TIGIT agent, an anti-TIM-3 agent, an anti-LAG-3 agent, an anti-BTLA agent, an anti-VISTA agent, an anti-B7-H3 agent, a 4-1BB agonist, an OX40 agonist, a CD40/CD40L agonist, an ICOS agonist, a GITR agonist or a CD28 agonist; and
(iii) a pharmaceutically acceptable carrier
to the subject in an amount sufficient to treat or prevent the neoplasia in the subject or a pharmaceutical composition for the treatment of neoplasia comprising:
(i) an EGFR inhibitor;
(ii) an anti-PD-1 agent, an anti-CTLA agent, an anti-KIR agent, an anti-TIGIT agent, an anti-TIM-3 agent, an anti-LAG-3 agent, a 4-1BB agonist, an ICOS agonist, a GITR agonist or a CD28 agonist and (iii) a pharmaceutically acceptable carrier.

48. The method of claim 47, wherein the neoplasia is selected from the group consisting of an ovarian cancer, a lung cancer, a colorectal cancer, a glioblastoma, a breast cancer, a prostate cancer, a renal cancer, a melanoma and a pancreatic cancer.

49. The method of claim 47, wherein the anti-PD-1 agent, anti-CTLA agent, anti-KIR agent, anti-TIGIT agent, anti-TIM-3 agent, anti-LAG-3 agent, 4-1BB agonist, ICOS agonist, GITR agonist or CD28 agonist is an antibody.

50. The method of claim 47, wherein the EGFR inhibitor is selected from the group consisting of erlotinib, gefitinib, afatinib and osimertinib.

51.-56. (canceled)

57. The method of claim 47, wherein the Noc4I inhibitor, Prpf19 inhibitor, Prmt5 inhibitor, Fbxw7 inhibitor, Eif3a inhibitor, Cd274 inhibitor, Mta2 inhibitor, Nat10 inhibitor and/or Map3k7 inhibitor is a CRISPR agent and/or an inhibitory nucleic acid.

58.-62. (canceled)

63. The method of claim 47, wherein the H2-K1 inhibitor, Hdac8 inhibitor, Tap1 inhibitor, Ep300 inhibitor, Tap2 inhibitor, Cbx5 inhibitor, B2m inhibitor, Brwd1 inhibitor, Cbx3 inhibitor and/or Chrac1 inhibitor is a CRISPR agent and/or an inhibitory nucleic acid.

64.-66. (canceled)

67. The method of claim 47, wherein the Noc4I inhibitor, Prpf19 inhibitor, Prmt5 inhibitor, Fbxw7 inhibitor, Eif3a inhibitor, Cd274 inhibitor, Mta2 inhibitor, Nat10 inhibitor and/or Map3k7 inhibitor is a CRISPR agent and/or an inhibitory nucleic acid.
